(19) United States
(12) Patent Application Publication
(10) Pub. No.: US 2023/0349073 A1
(43) Pub. Date: Nov. 2, 2023

(54) METHODS AND SYSTEMS FOR DETECTING TISSUE CONDITIONS

(71) Applicant: Molecular Stethoscope, Inc., San Diego, CA (US)

(72) Inventors: Michael Nerenberg, Del Mar, CA (US); Lian Chye Winston Koh, Palo Alto, CA (US)

(21) Appl. No.: 17/991,739

(22) Filed: Nov. 21, 2022

Related U.S. Application Data
(63) Continuation of application No. 17/724,971, filed on Apr. 20, 2022, now abandoned.
Application No. 17/724,971 is a continuation of application No. 16/832,449, filed on Mar. 27, 2020, now abandoned.
Application No. 16/832,449 is a continuation of application No. 16/082,380, filed on Sep. 5, 2018, now abandoned.
(60) Provisional application No. 62/408,566, filed on Oct. 14, 2016.
(60) Provisional application No. 62/334,621, filed on May 11, 2016.
(60) Provisional application No. 62/305,879, filed on Mar. 9, 2016.

(51) Int. Cl. C40B 40/08; C12Q 1/6883; C40B 50/00; C40B 40/06; C40B 40/10; G01N 33/68

(52) U.S. Cl. CPC C40B 40/08; C12Q 1/6883; C40B 50/00; C40B 40/06; C40B 40/10; G01N 33/6893; C12Q 2600/158

(57) ABSTRACT

Provided herein are methods and systems for detecting tissue conditions. In some aspects, levels of at least one marker of a disease or condition and at least one tissue-specific cell-free polynucleotide are quantified, levels are compared to a reference, and it is determined whether the tissue has been damaged by the disease or condition based on the comparing. Systems for performing the methods described herein are also provided.

### CROSS-REFERENCE

This application is a continuation of U.S. patent application Ser. No. 17/724,971, filed Apr. 20, 2022, which is a continuation of U.S. patent application Ser. No. 16/832,449, filed Mar. 27, 2020, which is a continuation application of U.S. patent application Ser. No. 16/082,380, filed Sep. 5, 2018, now abandoned, which is a National Phase Entry of International Application Serial No. PCT/US17/21637, filed Mar. 9, 2017, which claims the benefit of U.S. Provisional Patent Application Ser. Nos. 62/408,566, filed Oct. 14, 2016, 62/334,621, filed May 11, 2016; and 62/305,879, filed Mar. 9, 2016, each of which is incorporated herein by reference in their entireties.

### BACKGROUND OF THE INVENTION

A variety of markers are available for detecting various conditions. However, many of these conditions are ones that can affect different tissues. Detecting markers of these conditions in circulation, such as in a blood sample, are not always helpful in identifying which tissue is affected. For example, generic markers for inflammation can indicate an inflammatory response somewhere in the body, but it may not be known which tissue is suffering the response, such as the liver, kidney, lungs, or joints. Tissue-specific tests, such as biopsies, are often invasive, carrying a risk of infection, and typically not comprehensive of the entire organ or tissue. Imaging techniques, such as MRIs and CT-scans, may be used to assess tissue health, but generally can only detect overt features and changes. Thus, these imaging techniques are generally not sensitive enough to pick up early onset of conditions or fairly recent developments of conditions.

### SUMMARY OF THE INVENTION

Provided herein are methods of detecting a cardiovascular disease (CVD) biosignature in a biological fluid from a human subject. Some such methods comprise the steps of measuring a marker level in the biological fluid, wherein the marker is selected from a cholesterol, a lipid, an inflammatory mediator, a lipid mediator, and a sterol mediator; and quantifying a quantity of cardiovascular ribonucleic acids (RNA) in the biological fluid, wherein a threshold marker level and a threshold quantity of liver RNA indicates a CVD biosignature. Various aspects of these methods are recited below. Various aspects are contemplated as distinct and in combination. Optionally the at least one marker comprises a polynucleotide or protein encoded by a gene selected from the group consisting of: TPH1, CNTN4, CASQ2, MYOCD, FHL5, ATRNL1, RPS6KA6, RYR2, NPR3, ACADL, PLCB4, ITLN1, FIBIN, SCRG1, MRAP2, CNN1, ANGPTL1, SLC22A3, PRUNE2, PLD5, NEGR1, SEMA3D, NPR1, PDZRN3, NPNT, PLN, MPP6, SBSPON, THRB, NEXN, TTLL7, PLIN2, CCR1, SELF, MMRN1, CD163, RGS1, NPL, CD180, C7, FPR3, ST8SIA2, ASB18, MYL3, PRSS42, LRRC10, TNNI3, MYL2, SMCO1, CCDC141, MYH7, RD3L, MYBPC3, TNNT2, SCN5A, GJA3, CSRP3, MT1HL1, MYOZ2, XIRP1, KLHL31, PLEKHA5, ANKRD46, PIK3R1, TPR, TRAK2, ALDH5A1, MGEA5, DUT, FAM134B, ARIH2, COL21A1,

CBLB, SOBP, SLC16A7, ANP32E, PCMTD2, and EMCN. In some cases, the cardiovascular disease is atheroma and the marker is a polynucleotide or protein encoded by a gene selected from the group consisting of: TPH1, CNTN4, CASQ2, MYOCD, FHL5, ATRNL1, RPS6KA6, NPR3, RYR2, ACADL, PLCB4, ITLN1, FIBIN, SCRG1, MRAP2, CNN1, ANGPTL1, SLC22A3, PRUNE2, PLDS, NEGR1, SEMA3D, NPR1, PDZRN3, NPNT, PLN, MPP6, SBSPON, THRB, NEXN, and TTLL7. In some cases, the cardiovascular disease is diabetic ischemic cardiomyopathy and the marker is a polynucleotide or protein encoded by a gene selected from the group consisting of: NPR3, PLEHA5, ANKRD46, PIK3R1, TPR, TRAK2, ALDH5A1, MGEA5, DUT, FAM134B, ARIH2, PIK3R1, COL21A1, CBLB, SOBP, SLC16A7, ANP32E, and PCMTD2. In some cases, the quantity of cardiovascular RNA is substantially greater than that of at least one reference subject that does not have CVD. In some cases, the quantity of cardiovascular RNA does not differ substantially from that of at least one reference subject that has CVD. In some cases, the methods comprise comparing the quantity of the cardiovascular RNA to an average cardiovascular RNA level in a plurality of subjects suffering from CVD. In some instances, the quantity of the cardiovascular RNA being equal to or greater than the average levels indicates the human subject suffers from CVD. In some cases, the methods comprise detecting the CVD biosignature when the quantity of the cardiovascular RNA is at least equal to or greater than those of at least one subject with CVD. In some cases, measuring the quantity of cardiovascular RNA to the biological fluid comprises measuring the relative contribution of cardiovascular RNA to total circulating ribonucleic acids. Optionally, the cardiovascular RNA does not encode proteins implicated in CVD. Optionally, the cardiovascular RNA does not encode proteins upregulated in a liver of a reference subject with CVD. In some cases, the quantity of the cardiovascular RNA not differing significantly from corresponding reference levels indicative of a reference cardiovascular health status indicates the human subject's cardiovascular health status is similar to the reference cardiovascular health status. Optionally, methods disclosed herein comprise obtaining a second biological fluid, and detecting a CVD biosignature in the second biological fluid. Optionally, the second biological fluid is obtained subsequent to a CVD intervention. Optionally, the CVD intervention comprises at least one of reducing alcohol intake, reducing caloric intake, increasing exercise, reducing cholesterol level, reducing inflammation and improving insulin sensitivity. Optionally, the CVD intervention comprises consuming a compound selected from the group consisting of: a cholesterol-regulating compound, a lipid-regulating compound, an anti-inflammatory compound, and an insulin sensitizing compound. In some cases, the cardiovascular RNA is RNA that is predominantly expressed in a tissue selected from the group consisting of: heart, aorta, coronary artery, vascular smooth muscle and endothelium. In some cases, the cardiovascular RNA is RNA expressed at a substantially higher level in a cardiovascular tissue than in any other tissue of the human subject. In some cases, the cardiovascular RNA is RNA that is predominantly expressed in coronary artery or aorta. Optionally, the cardiovascular RNA is RNA that is predominantly expressed in cells selected from endothelial cells, vascular smooth muscle cells, renal cells and cardiomyocytes. Optionally, the cardiovascular RNA corresponds to a gene selected from the group consisting of: ACTC1, ANKRD1, ASB18, BMP10, CASQ2, CCDC141, CHRNE, CORIN, CSRP3, DAND5, FABP3, GJA3, KLHL31, LRRC10, MT1HL1, MYBPC3, MYBPHL, MYH6, MYH7, MYL2, MYL3, MYL4, MYL7, MYOZ2, MYZAP, NPPA, NPPB, PLN, POPDC2, PPP1R1C, PRSS42, RD3L, RMB20, RYR2, SBK2, SBK3, SCN5A, SMCO1, ST8SIA2, TBX20 TECRL, TNNI3, TNNI3K, TNNT2, and XIRP1. In some cases, the cardiovascular RNA is coronary artery RNA and corresponds to a gene selected from the group consisting of: CNTN4, CASQ2, MYOCD, FHL5, NPR3, ACADL, FIBIN, MRAP2, CNN1, SLC22A3, SEMA3D, NPR1, NPNT, PLN, SBSPON, C7, and FPR3. Optionally, methods disclosed herein comprise measuring a quantity of deoxyribonucleic acids (DNA) in the biological fluid, wherein the DNA has a cardiovascular methylation pattern of at least one locus. In some instances, the quantity of DNA having a cardiovascular methylation pattern is substantially higher than that of at least one reference subject that does not have CVD. In some instances, the quantity of DNA having a cardiovascular methylation pattern does not differ substantially from that of at least one reference subject that has CVD. In some instances, measuring the quantity of DNA having a cardiovascular methylation pattern of at least one locus to the biological fluid comprises measuring the relative contribution of DNA having a cardiovascular methylation pattern of at least one locus to total DNA in the biological fluid. In some instances, the at least one locus of the methylated DNA is not implicated in CVD. In some instances, the at least one locus of the methylated DNA is not differentially methylated between a healthy cardiovascular tissue and a cardiovascular tissue affected by CVD. Optionally, methods disclosed herein comprise comparing methylation status of at least one locus of the methylated DNA to a reference, wherein methylation above a threshold indicates an overrepresentation of cardiovascular DNA in the biological fluid. Optionally, methods disclosed herein comprise sequencing at least one DNA loci and at least one RNA in the biological fluid. In some cases, the biological fluid is plasma or serum. In some cases, the cardiovascular RNA is freely circulating RNA.

Also provided herein are methods of detecting a non-alcoholic steatohepatitis (NASH) biosignature in a biological fluid from a human subject. Some such methods comprise the steps of measuring a marker level in the biological fluid, wherein the marker is selected from a cholesterol, a lipid, an inflammatory mediator, a lipid mediator, and a cholesterol mediator; and measuring a quantity of liver ribonucleic acids (RNA) in the biological fluid; wherein a threshold marker level and a threshold quantity of liver RNA indicates a NASH biosignature. Various aspects of these methods are recited below. Various aspects are contemplated as distinct and in combination. In some cases, the marker comprises at least one polynucleotide or protein encoded by a gene selected from the group consisting of: LXR-alpha, PPAR-gamma, SREBP-1c, SREBP-2, FAS, iNOS, COX2, OPN, TFN-alpha, SOCS3, IL6, and PNPLA3 I148M. Optionally, the cholesterol mediator is selected from a polynucleotide or protein encoded by a gene selected from the group consisting of: LXR-alpha, SREBP-1c, and SREBP-2. Optionally, the inflammatory mediator is a polynucleotide or protein encoded by a gene selected from the group consisting of: iNOS, COX2, OPN, TFN-alpha, SOCS3 and IL-6. Optionally, the lipid mediator is selected from a polynucleotide or protein encoded by a gene selected from the group consisting of: PPAR-gamma, FAS, and PNPLA3 I148M. In some instances, the threshold quantity of liver RNA is substantially greater than that of at least one reference subject that does not have NASH. In some instances, the threshold quantity of liver RNA does not differ substantially from that of at least one reference subject that has NASH. In some cases, methods disclosed herein comprise comparing the quantity of liver RNA to respective reference levels, wherein the respective reference levels are average levels in a plurality of subjects suffering from NASH. In some instances, the threshold quantity of liver RNA being equal to or substantially greater than the average levels indicates the human subject suffers from NASH. In some cases, methods disclosed herein comprise detecting the NASH biosignature when the threshold quantity of liver RNA is at least equal to or substantially greater than those of at least one subject with NASH. Optionally, methods disclosed herein comprise measuring the quantity of liver RNA comprises measuring the relative contribution of liver RNA to a nucleic acid population selected from total RNA of the biological fluid and total nucleic acids of the biological fluid. Optionally, liver RNA disclosed herein does not encode proteins implicated in NASH. Optionally, liver RNA disclosed herein does not encode proteins upregulated in a liver of a reference subject with NASH. In some instances, the quantity of liver RNA not differing significantly from corresponding reference levels indicative of a reference liver health status indicates the human subject's liver health status is similar to the reference liver health status. Optionally, methods disclosed herein comprise obtaining a second biological fluid, and detecting a NASH biosignature in the second biological fluid. Optionally, the second biological fluid is obtained subsequent to a NASH intervention. In some cases, NASH intervention comprises at least one of reducing alcohol intake, reducing caloric intake, increasing exercise, undergoing gastric bypass surgery, reducing cholesterol level, reducing inflammation and improving insulin sensitivity. In some cases, NASH intervention comprises consuming a compound selected from a cholesterol-regulating compound, an anti-inflammatory compound, and an insulin sensitizing compound. In most cases, liver RNA disclosed herein is RNA that is predominantly expressed in a human liver. In most cases, liver RNA disclosed herein is RNA expressed substantially higher in liver than in any other tissue of the human subject. In some cases, liver RNA corresponds to a gene selected from the group consisting of: 1810014F10RIK, A1BG, ABCC2, ABCC6, ABCG5, ANG, ANGPTL3, ACOX2, ACSM2A, ADH1A, ADH1C, ADH6, AFM, AFP, AGXT, AHSG, AKR1C4, AKR1D1, ALB, ALDH1B1, ALDH4A1, ALDOB, AMBP, AOC3, APCS, APOA1, APOA2, APOA5, APOB, APOC1, APOC2, APOC3, APOC4, APOE, APOF, APOH, APOM, ARID1A, ARSE, ASL, AQP9, ASGR1, ASGR2, ATF5, C4A, C4BPA, C6, C8A, C8B, C8G, C9, CAPN5, CES1, CES2, CFHR1, CFHR2, CFHR3, CFHR4, CFHR5, CHD2, CIDEB, CPN1, CRLF1, CRYAA, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F12, DIO1, DAK, DCXR, F10, F12, F2, F9, FAH, FCN2, FETUB, FGA, FGB, FGG, FMO3, FTCD, G6PC, GPC3, GALK1, GAMT, GBA, GBP7, GCKR, GLYAT, GNMT, GPT, GSTM1, HAAO, HAMP, HAO1, HGD, HGFAC, HMGCS2, haptoglobin, HPN, HPR, HPX, HRG, HSD11B1, HSD17B6, HLF, IGF2, IL1RN, IGFALS, IQCE, ITIH1, ITIH2, ITIH4, JCLN, KHK, KLK13, LBP, LECT2, LOC55908, LPA, MASP2, MBL2, MGMT, MUPCDH, NHLH2, NNMT, NSFL1C, OATP1B1, ORM2, PCK1, PEMT, PGC, PLG, PKLR, PLGLB2, POLR2C, PON1, PON3, PROC, PXMP2, RBP4, RDH16, RET, SAA4, SARDH, SDS, SDSL, SEC14L2, SERPINA4, SERPINA7, SERPINA10, SERPINA11, SERPINC1, SERPIND1, SLCO1B1, SLC10A1, SLC22A1, SLC22A7, SLC22A10, SLC25A47, SLC27A5, SLC38A3, SLC6A12, SPP2, TAT, TBX3, TF, TIM2, TMEM176B, TST, UPB1, UROC1, VTN, WNT7A, C2, C2ORF72, CPB2, CYP4F11, CYP4F2, DUSP9, GABBR1, HP, HPD, IGSF1, IL17RB, ITIH2, ITIH3, LCAT, LGALS4, MAT1A, MST1, MSTP9, NR0B2, NR1I2, ORM1, RELN, RGN, RHBG, SAA4, SERPINA5, SERPINA7, SERPINC1, SERPINF2, SLC2A2, SULT1A2, SULT2A1, TCP10L, TNNI2, UGT2B15, and UGT2B17. Optionally, measuring a quantity of a deoxyribonucleic acid (DNA) in the biological fluid, wherein the DNA has a liver methylation pattern of at least one locus. In some cases, the quantity of DNA having a liver methylation pattern is substantially greater than that of at least one reference subject that does not have NASH. In some instances, the quantity of DNA having a liver methylation pattern does not differ substantially from that of at least one reference subject that has NASH. Optionally, measuring the quantity of DNA having a liver methylation pattern of at least one locus comprises measuring the relative contribution of DNA having a liver methylation pattern of at least one locus to total DNA in the biological fluid. In some cases, the at least one locus of the methylated DNA is not implicated in NASH. In some cases, the at least one locus of the methylated DNA is not differentially methylated between a healthy liver tissue and a liver affected by NASH.

In some instances, methylation above a threshold indicates an overrepresentation of liver DNA in the biological fluid. Optionally, methods disclosed herein comprise sequencing at least one DNA loci and at least one RNA in the biological fluid. In some cases, biological fluid is plasma or serum. In most instances, the liver RNA is freely circulating RNA.

Also provided herein are methods of monitoring a human subject with a chronic metabolic condition for a presence or increased risk of at least one complication at least one tissue. Some such methods comprise the steps of: obtaining a biological fluid from the subject; measuring a marker level in the biological fluid, wherein the marker is selected from a cholesterol, a lipid, insulin, an inflammatory mediator, a lipid mediator, an insulin mediator and a cholesterol mediator; and quantifying ribonucleic acids (RNA) in the biological fluid from liver, cardiovascular tissue, and kidney, wherein a threshold marker level and a threshold quantity of the RNA indicates the presence or increased risk of the complication in at least one of the liver, cardiovascular tissue and kidney. Various aspects of these methods are recited below. Various aspects are contemplated as distinct and in combination. In some cases, the at least one complication is selected from the group consisting of: NASH, liver fibrosis, liver cirrhosis, liver failure, diabetic nephropathy, renal ischemia, renal fibrosis, kidney failure, atherosclerosis, diabetic cardiomyopathy, atheroma, coronary artery disease, myocardial infarction, stroke and aneurysm. In many instances, the chronic metabolic condition is selected from the group consisting of: obesity, type II diabetes and NAFLD. In some cases, the threshold quantity of RNA is substantially greater than that of at least one reference subject that does not have the at least one complication. In some cases, the threshold quantity of RNA does not differ substantially from that of at least one reference subject that has the at least one complication. Optionally, methods disclosed herein comprise comparing the threshold quantity of RNA to respective reference levels, wherein the respective reference levels are average levels in a plurality of subjects suffering the at least one complication. In some cases, the threshold quantity of RNA being equal to or substantially greater than the average levels indicates the human subject suffers from the at least one complication. Optionally, methods disclosed herein comprise detecting the complication when the threshold quantity of RNA is at least equal to or substantially greater than those of at least one subject with the at least one complication. Optionally, biological fluids are selected from the group consisting of: plasma, urine and saliva. Optionally, methods disclosed herein comprise measuring a marker level in whole blood and quantifying relative contributions of RNA in a plasma fraction of the whole blood. In most cases, the RNA is freely circulating RNA. In some instances, the inflammatory mediator is a cytokine. In some instances, the cholesterol mediator is a protein that mediates cellular uptake of cholesterol, cellular efflux of cholesterol, cholesterol metabolism, or modifications of cholesterol. In some cases, the lipid mediator is a mediator of lipid metabolism, lipid trafficking, lipid storage, or modifications of lipids. Optionally, RNA from kidney corresponds to a gene selected from the group consisting of: AK3L1, AQP2, AQPN6, ATP6V1G3, ATP6V0D2, BBOX1, BFSP2, BHMT, BSND, C20ORF194, C9orf66, CALB1, CA12, CDH16, CLCNKA, CRYAA, CRYBB3, CTXN3, CUBN, CYS1, DDC, DNMT3L, EGF, ENPEP, FCAMR, FMO1, FOLR3, FUT3, FXYD2, FXYD4, GGT1, HAO2, HAVCR1, HKID, HMX2, HNF1B, KAAG1, KCNJ1, KL, MCCD1, MIOX, NAT8, NOX4, NPHS2, OR2T10, PAX2, PDZK1, PDZK1IP1, PRR35, PTH1R, RBP5, SIM1, SLC12A1, SLC12A3, SLC13A3, SLC17A3, SLC22A11, SLC22A12, SLC22A13, SLC22A2, SLC22A24, SLC22A6, SLC22A8, SLC22A13, SLC34A1, SLC3A1, SLC4A9, SLC5A2, SLC5A10, SLC6A13, SLC6A18, SLC7A7, SLC7A8, SLC7A9, SOST, TREH, TMEM27, TMEM52B, TMEM72, TMEM174, TMEM207, UGT1A1, UGT1A6, UGT1A9, UMOD, UPP2, XPNPEP2, and 0001T8. Optionally, RNA from liver corresponds to a gene selected from the group consisting of: 1810014F10RIK, A1BG, ABCC2, ABCC6, ABCG5, ANG, ANGPTL3, ACOX2, ACSM2A, ADH1A, ADH1C, ADH6, AFM, AFP, AGXT, AHSG, AKR1C4, AKR1D1, ALB, ALDH1B1, ALDH4A1, ALDOB, AMBP, AOC3, APCS, APOA1, APOA2, APOA5, APOB, APOC1, APOC2, APOC3, APOC4, APOE, APOF, APOH, APOM, ARID1A, ARSE, ASL, AQP9, ASGR1, ASGR2, ATF5, C4A, C4BPA, C6, C8A, C8B, C8G, C9, CAPN5, CES1, CES2, CFHR1, CFHR2, CFHR3, CFHR4, CFHR5, CHD2, CIDER, CPN1, CRLF1, CRYAA, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F12, DIO1, DAK, DCXR, F10, F12, F2, F9, FAH, FCN2, FETUS, FGA, FGB, FGG, FMO3, FTCD, G6PC, GPC3, GALK1, GAMT, GRA, GBP7, GCKR, GLYAT, GNMT, GPT, GSTM1, HAAO, HAMP, HAO1, HGD, HGFAC, HMGC52, haptoglobin, HPN, HPR, HPX, HRG, HSD11B1, HSD17B6, HLF, IGF2, IL1RN, IGFALS, IQCE, ITIH1, ITIH2, ITIH4, JCLN, KHK, KLK13, LBP, LECT2, LOC55908, LPA, MASP2, MBL2, MGMT, MUPCDH, NHLH2, NNMT, NSFL1C, OATP1B1, ORM2, PCK1, PEMT, PGC, PLG, PKLR, PLGLB2, POLR2C, PON1, PON3, PROC, PXMP2, RBP4, RDH16, RET, SAA4, SARDH, SDS, SDSL, SEC14L2, SERPINA4, SERPINA7, SERPINA10, SERPINA11, SERPINC1, SERPIND1, SLCO1B1, SLC10A1, SLC22A1, SLC22A7, SLC22A10, SLC25A47, SLC27A5, SLC38A3, SLC6A12, SPP2, TAT, TBX3, TF, TIM2, TMEM176B, TST, UPB1, UROC1, VTN, WNT7A, C2, C2ORF72, CPB2, CYP4F11, CYP4F2, DUSP9, GABBR1, HP, HPD, IGSF1, IL17RB, ITIH2, ITIH3, LCAT, LGALS4, MAT1A, MST1, MSTP9, NR0B2, NR1I2, ORM1, RELN, RGN, RHBG, SAA4, SERPINA5, SERPINA7, SERPINC1, SERPINF2, SLC2A2, SULT1A2, SULT2A1, TCP10L, TNNI2, UGT2B15, and UGT2B17. Optionally, RNA from cardiovascular tissue corresponds to a gene selected from the group consisting of: ACTC1, ANKRD1, ASB18, BMP10, CASQ2, CCDC141, CHRNE, CORIN, CSRP3, DAND5, FABP3, GJA3, KLHL31, LRRC10, MT1HL1, MYBPC3, MYBPHL, MYH6, MYH7, MYL2, MYL3, MYL4, MYL7, MYOZ2, MYZAP, NPPA, NPPB, PLN, POPDC2, PPP1R1C, PRSS42, RD3L, RMB20, RYR2, SBK2, SBK3, SCN5A, SMCO1, ST8SIA2, TBX20 TECRL, TNNI3, TNNI3K, TNNT2, and XIRP1. In some cases, monitoring comprises performing the steps of measuring a marker level in the biological fluid, wherein the marker is selected from a cholesterol, a lipid, an inflammatory mediator, a lipid mediator, and a cholesterol mediator; and measuring a quantity of liver ribonucleic acids (RNA) in the biological fluid at least one time. In some cases, monitoring comprises performing the steps of measuring a marker level in the biological fluid, wherein the marker is selected from a cholesterol, a lipid, an inflammatory mediator, a lipid mediator, and a cholesterol mediator; and measuring a quantity of liver ribonucleic acids (RNA) in the biological fluid at a first time point and a second time point. In some instances, no presence or risk of complications are detected at the first time point. In other instances, a presence or risk of at least one complication of at least one organ of the multiple organs is detected at the first time point, and the second time point occurs subsequent to an intervention or treatment of the complication.

Further provided herein are systems. Such systems comprise: (a) a memory unit configured to store results of (i) an assay for detecting at least one marker of each of at least one condition in a first sample of a subject, and (ii) an assay for detecting at least one tissue-specific RNA in a second sample of a subject, wherein each of the at least one tissue-specific RNA is a cell-free RNA specific to a tissue; (b) at least one processor programmed to: (i) quantify a level of the at least one marker; (ii) quantify a level of the at least one tissue-specific polynucleotide; (iii) compare the level of each of the at least one marker to a corresponding reference level of the marker; (iv) compare the level of each of the at least one tissue-specific polynucleotide to a corresponding reference level of the tissue-specific polynucleotide; and (v) determine presence of or relative change in damage of the tissue by the at least one condition based on the comparing; and (c) an output unit that delivers a report to a recipient, wherein the report provides results generated by the processor in (b). Optionally, reports comprise a recommendation for medical action based on the generated by the processor in (b). In some instances, medical action comprises recommended treatment. In many instances, the at least one tissue-specific polynucleotide comprises at least one tissue specific RNA.

In some instances, the at least one tissue-specific polynucleotide comprises at least one tissue-specific methylated DNA, wherein each tissue-specific methylated DNA comprises a tissue-specific methylation pattern. Optionally, the tissue is determined to be damaged by the condition if (a) the level of at least one of the marker is above the reference level of the at least one marker, and (b) the level of at least one of the tissue-specific polynucleotide is above the reference level of the at least one tissue-specific polynucleotide. In some cases, the at least one condition is at least one of: inflammation, apoptosis, necrosis, fibrosis, infection, autoimmune disease, arthritis, liver disease, neurodegenerative disease, and cancer. In some instances, the at least one condition comprises multiple sclerosis. Optionally, the condition is inflammation, and the at least one marker corresponds to a gene selected from the group consisting of: AHSG, APCS, COX2, FAS, IL6, iNOS, OPN, ORM1, SIGIRR, SOCS3, TFN-alpha, and combinations thereof. Optionally, the condition is fibrosis, and the at least one marker corresponds to a gene selected from the group consisting of: ALT, AST, C4M CPK, CO3-610, C06-MMP, CO1-764, CTGF, IL-4, IL-6, IL-8, IL-18 MFAP, MMP1, MMP2, MMP9, MMP13, PDGF, PIIINP, PINP, P4NP 7S, PVCP, TGF-beta, TIMP1, TIMP2, TIMP3, TNF-alpha, YKL40, a gene encoding a troponin, and a gene encoding type IV collagen, and combinations thereof. Optionally, the condition is apoptosis, and the at least one marker corresponds to a gene selected from the group consisting of: ALB, APAF1, APOE, CFLAR, CIDEB, F2, PLG, PROC, and TNFSF18, and combinations thereof. In some cases, the condition is liver disease. In some instances, the liver disease is non-alcoholic fatty liver disease, non-alcoholic steatosis, or non-alcoholic steatohepatitis. In some cases, the liver disease is non-alcoholic fatty liver disease, and the method further comprises determining progress, or a lack thereof, toward non-alcoholic steatohepatitis, based on the report. In some instances, the at least one marker corresponds to a gene selected from the group consisting of: COX2, FAS, IL6, iNOS, LXR-alpha, OPN, PNPLA3 I148M, PPAR-gamma, SOCS3, SREBP-1c, SREBP-2, and TFN-alpha, and combinations thereof. In some instances, the at least one marker is selected from the group consisting of: CRP, FIGF, HGF, ICAM1, IL2, IL2RA, IL8RB, KRT18, PI3, REG3A, ST2, TIMP1, TNFR, and TNFRSF1A, and combinations thereof. In some instances, the at least one marker is cell-free RNA.

### BRIEF DESCRIPTION OF THE DRAWINGS

A better understanding of the features and advantages of the present disclosure is obtained by reference to the following detailed description that sets forth illustrative embodiments, in which the principles of the disclosure are utilized, and the accompanying drawings of which:

FIG. 1 shows an illustration of a system according to an embodiment.

FIG. 2 depicts exemplary relative contributions of tissue-specific polynucleotides according to an embodiment.

### DETAILED DESCRIPTION OF THE INVENTION

Methods, systems and kits described herein relate to the rapid, noninvasive detection of disorders using a combination of marker types so as to concurrently determine both a likely disorder and a likely tissue under duress. Through practice of the disclosure herein, one is able to make confident predictions as to a disease identity and the extent of its impact on one or more tissues, without requiring any invasive investigation of the tissue or tissues suspected of being impacted.

Often but not exclusively, one of the markers is circulating RNA that can be readily correlated to a tissue of origin, such that an increase in the relative contribution of RNA from that organ is indicative of duress in or specific to that organ. Single markers and aggregate RNA derived from an organ are both contemplated in various embodiments as indicators of tissue status. Alternately or in combination, circulating DNA, such as DNA that is differentially methylated in a tissue-specific manner, is included as part or all of a tissue-specific marker.

Concurrently, markers indicative of a type of disorder are also measured. There is a broad range of markers contemplated as indicative of a type disorder, including proteins, steroids, lipids, cholesterols, or nucleic acids such as DNA or RNA. RNA such as particular transcripts encoding proteins implicated in a disease or disorder are particularly useful, as are DNA having methylation patters that are indicative of a disease state. Often but not always, the disease marker is also a circulating marker that is readily obtained from, for example, a blood draw. However, alternatives such as X-ray, MRI or other data are contemplated as markers for some diseases.

By comparing the levels or identities of these markers to reference values or datasets, one may categorize a patient or a patient's sample as being indicative of a particular disease in the patient, localized as a particular tissue or organ. The reference values or datasets will vary as to the disease and tissue, and will variously include data from one or more healthy individuals, one or more individuals suffering from various extents of a disorder or tissue duress, data from intermediate individuals, and data predicted from models. A sample can be categorized as indicative of a disorder or condition when its values are individually or collectively above or below a threshold, or when they do not differ significantly from a reference data set correlated with the disorder or condition, or when they do differ significantly from a reference dataset correlated with absence of the disease or disorder.

For instance, methods, systems and kits described herein may be used to screen for development or progression of a condition, or multiple conditions, in multiple organs, in an at-risk population on a routine basis. This can be especially useful in subjects with chronic conditions, such as metabolic syndrome, obesity, diabetes, neurodegenerative disorders and cancer, where one or more tissues are at risk of injury, damage or failure.

Metabolic syndrome and obesity affect a large and ever-growing percentage of the population worldwide. This population is at a constant and relatively high risk of developing life-threatening complications, such as heart attack, stroke, liver cirrhosis, pancreatic exhaustion, and kidney failure. Thus, this population is at a constant risk of developing complications in an array of organs and tissues. Similarly, many cancers are at a constant risk of mutating and metastasizing to different tissues and organs. In addition, treatments for cancer are often administered with uncertainty of success, and it is desirable to rapidly determine whether or not these treatments are effective or toxic. In these exemplary cases, it is not practical to assess subjects on a routine basis using traditional methods, such as imaging techniques and biopsies. However, methods, systems and kits, such as those described herein, provide for rapidly detecting insult, increased risk and therapeutic effects in one or more organs in a subject, thereby providing a means to monitor subjects with chronic conditions for acute complications, disease progression, and therapeutic effects.

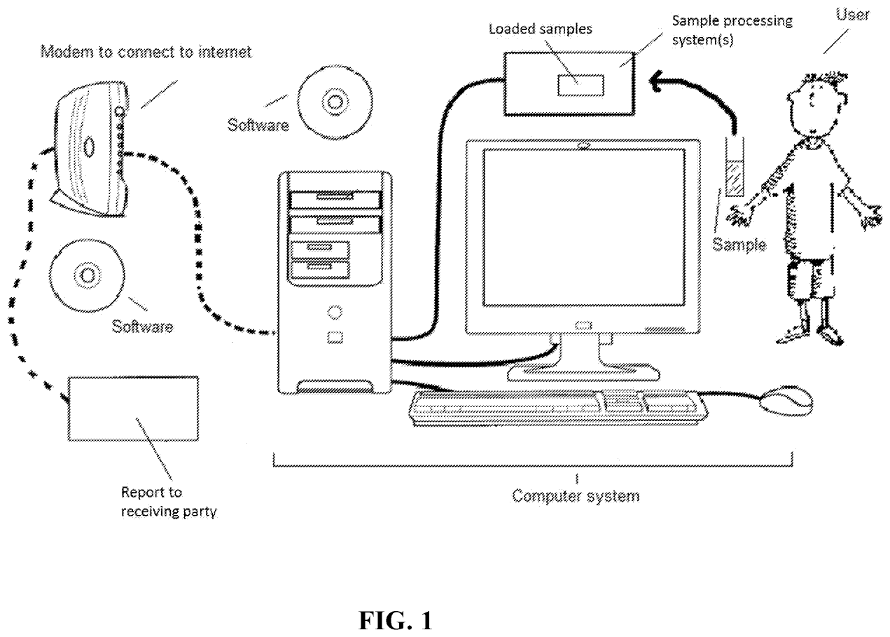

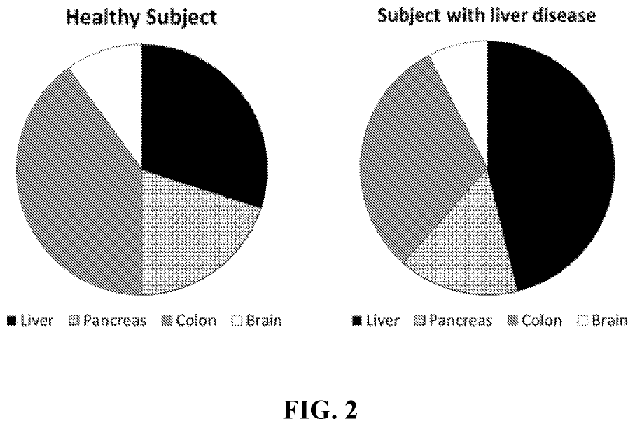

The following methods, kits, and systems are intended to rapidly and non-invasively detect tissues or organs in a subject that are under duress, damaged or affected by a condition or disease. In some instances, the following methods, kits, and systems also determine which disease or condition is affecting the tissue under duress or to what extent the disease or condition is affecting the tissue. As shown in FIG. 1, a sample, such as blood plasma, saliva, or urine, is collected from the subject and analyzed for markers and cell-free polynucleotides that can indicate disease and disease location in the subject. These methods, kits and systems generally rely on circulating, cell-free nucleic acids that are released or secreted from the tissue or organ under duress into biological fluids, such as cell-free RNA in a plasma or urine sample. By focusing on genes that are specifically expressed or predominantly expressed in a certain tissue, inferences or conclusions can be drawn about the health status of that tissue based on the relative contribution of RNA from the tissues to total circulating RNA. By quantifying relative contributions, as represented for example, in FIG. 2, one can advantageously locate tissues affected by a condition without invasive biopsies or macroscale-limited imaging techniques. Tissue-specific nucleic acids are used in combination with markers for various conditions to select therapies, monitor effects of therapies, and monitor progression of a disease or condition.

Identifying diseases and tissues under duress may require comparing levels of tissue-specific polynucleotides and markers in a sample of a test subject to those of at least one sample from a control subject. The tissue-specific polynucleotides and markers may be referred to as a panel herein. In some instances, levels of markers and tissue-specific polynucleotides in a sample that is obtained from a test subject are compared to those of a control subject. In some instances, levels of markers and tissue-specific polynucleotides in a sample that is obtained from a test subject are compared to an average of corresponding levels in multiple control subjects. The control subjects may have a condition of interest or the control subjects may be subjects without the condition.

Methods, systems and kits provide for detecting or quantifying a panel of tissue-specific polynucleotides and/or markers. It is recognized that gene expression may vary tremendously within a population of subjects and between populations of subjects (e.g., between different ethnic groups), and in such cases, a panel of tissue-specific polynucleotides and/or markers may be particularly useful. For instance, the methods may comprise comparing the panel to at least one control panel. While the expression levels of each tissue-specific polynucleotide and marker may not be similar, a conclusion or inference can still be made about the condition or tissue(s) of the subject if the panel is sufficiently similar or sufficiently different from a control panel. In this way a panel may provide an advantage over using a single marker of disease or a single tissue-specific polynucleotide. In some instances, the methods comprise comparing the panel of a subject at a first time point to the panel of the subject at a second time point. Thus, a single subject's natural genetic variations and gene expression fluctuations are controlled for and differences between panels are more likely due to changes in the condition or tissue(s) affected. In some instances, the panel may comprise non-polynucleotide molecules. The panel may comprise polynucleotides and other biological molecules (e.g., peptides, lipids, pathogen fragments, etc.).

Methods, kits, and systems described herein may be used to determine the likelihood or risk of the subject developing the disease or condition, the progression or severity of the disease or condition, or the effect of a therapy or treatment on the disease or condition. Kits, systems and methods disclosed herein are sensitive and accurate enough to compare a first level of a marker or tissue-specific polynucleotide to a second level of the marker or tissue-specific nucleic acid, in order to differentiate between a risk of a condition, a progressed state of a condition, or an improvement of a condition by a treatment. In some instances, the first level of the marker or tissue-specific nucleic acid corresponds to a sample from a subject at a first time point and the second level of the marker or tissue-specific nucleic acid corresponds to a second sample from a subject at a second time point.

Multiple diseases and tissues may be assessed simultaneously using the kits, systems and methods disclosed herein. In this way, the kits, systems and methods disclosed herein may be used to assess the presence or absence of at least one condition and identify both affected and unaffected tissues. In some embodiments, methods comprise selecting or recommending a medical action based on results produced by the methods, systems or kits disclosed herein. In some embodiments, a customized medical action is recommended, and optionally taken, based on the determination. In some instances, customized medical action comprises directly treating a tissue under duress, e.g., with radiation or injection of the tissue. Non-limiting examples of medical actions include performing additional tests (e.g., biopsy, imaging, surgery), treating the subject for the disease or condition, and modifying a treatment of the subject (e.g. altering the dose of a pharmaceutical composition, ceasing administration of a pharmaceutical composition, administering a different or additional pharmaceutical composition).

The systems, methods and kits disclosed herein may provide for detecting a condition or disease in multiple tissues. In some instances, a subject has a condition known to affect one or more tissues depending on the extent or severity of the condition. Systems, methods and kits, such as those disclosed herein, advantageously allow for identification and targeted treatment of multiple tissues under duress. For example, a system disclosed herein may provide markers for detecting inflammation in a subject and determining that the liver and heart are affected by the inflammation due to the levels of circulating liver-specific RNAs and heart-specific RNAs. Also, by way of example, the methods may comprise detecting cell-free RNA in a plasma sample that harbor mutations associated with cancer (e.g., mutations that occur as a cause or consequence of cancer), or that is present at a level indicative of cancer. Once the presence of a cancer is detected, the methods may further comprise quantifying tissue-specific, relative contributions cell-free RNAs from various tissues to determine which tissues may be harboring a tumor, or beginnings thereof.

In addition to detecting tissues that are damaged, the methods further provide for identifying, or differentiating between, conditions that are causing the tissue damage. By way of non-limiting example, methods are disclosed herein for detecting liver damage in a subject, identifying a condition causing the liver damage, selecting a therapy to treat the subject and monitoring the effectiveness of the therapy. Cell-free RNA that corresponds to genes predominantly expressed in the human liver is quantified in a plasma sample of a subject. Elevated levels of such RNA in the plasma sample indicate there is liver damage. Identifying, or differentiating between, diseases, as described herein, generally depends on quantifying, not merely detecting, the tissue-specific RNA and quantifying markers of disease. For example, non-alcoholic fatty liver disease (NAFLD) and the more progressed and severe disease, non-alcoholic steatohepatitis (NASH) may be identified by similar liver-specific RNA and markers, but levels of these molecules may be higher in cases of NASH than in cases of NAFLD because there is more liver damage occurring in NASH than NAFLD. Since more damage of the liver occurs in NASH than NAFLD in most cases, more liver-specific RNA will be released from the liver in a case of NASH than in a case of NAFLD.

Disease presence and location in a subject can be determined at an early stage of disease, because the systems and methods described herein provide rapid results, are non-invasive and inexpensive. Thus, the subject can be advantageously treated before the disease progresses to advanced stages that are relatively more difficult to control or treat as compared to early stages. For example, the systems and methods disclosed herein may allow for determining which tissue(s) or organ(s) have cancerous cells before a tumor is large enough to be visualized with an imaging technique, such as a CT or PET scan. In this way, the methods and systems disclosed herein provide for focused analysis and targeted therapies, such as local injection and targeted radiation, at early stages of disease.

Advantageously, the methods and systems provide for treating with a therapy that is suitable or optimal for the extent of tissue damage. In some instances, the methods comprise detecting/quantifying the markers and/or tissue-specific polynucleotides to assess the effectiveness or toxicity of a therapy. In some instances the therapy is continued. In other instances, the therapy is discontinued and/or replaced with another therapy. Regardless, due to the rapid and non-invasive nature of the methods and systems, therapeutic effects can be assessed and optimized more often relative to conventional treatment optimization.

By way of non-limiting example, according to conventional practice, a patient being treated for cancer is administered a chemotherapy and an MRI is performed three months later to determine if tumor size is reduced. When an increase in tumor size is observed, the medical practitioner prescribes a different therapy, but the tumor has already metastasized. In contrast, using methods described herein, the patient would be tested one to two weeks after initiating treatment to assess levels of tissue-specific nucleic acids corresponding to tissues harboring tumors as well as markers of treatment effectiveness. When levels of tissue-specific nucleic acids and markers indicate the therapy is ineffective, the practitioner prescribes a different therapy that is quickly determined, by similar methods, to be effective. In the latter case, tumors have less time to grow relative to the conventional method that utilizes imaging techniques and do not metastasize, thereby providing the patient with a better prognosis.

In some aspects, the present disclosure provides for uses of systems, samples, markers, and tissue-specific polynucleotides disclosed herein. In some instances, disclosed herein are uses of an in vitro sample for non-invasively detecting a tissue or organ in a subject that is under duress and as well as a disease or condition that is the cause of the duress. In some instances, disclosed herein are uses of an ex vivo sample for non-invasively detecting a tissue or organ in a subject that is under duress and as well as a disease or condition that is the cause of the duress. Generally, uses disclosed herein comprise quantifying markers and tissue-specific polynucleotides in samples, including ex vivo samples and in vitro samples. Some uses disclosed herein comprise comparing a quantity of a marker and a quantity of tissue-specific polynucleotide in a first sample and comparing the quantities to respective quantities in a second sample. In some instances, the first sample is from a first subject and the second sample is from a control subject (e.g., a healthy subject or subject with a condition). In some instances, the first sample is from a subject at a first time point and the second sample is from the same subject at a second time point. The first time point may be obtained before the subject is administered a therapy and the second time point may be obtained after the therapy. Thus, also provided herein are uses of samples, markers, tissue-specific polynucleotides, kits and systems disclosed herein to monitor or evaluate a condition of a subject, tissue health state of a subject, or an effect of a therapeutic agent.

### Certain Terminologies

The following descriptions are provided to aid the understanding of the methods, systems and kits disclosed herein. The following descriptions of terms used herein are not intended to be limiting definitions of these terms. These terms are further described and exemplified throughout the present application.

Methods, systems and kits described herein generally detect and quantify cell-free nucleic acids. For this reason, biological samples described herein are generally acellular biological fluids. Samples from subjects, by way of non-limiting example, may be blood from which cells are removed, plasma, serum, urine, or spinal fluid. For instance, the biological molecule may be circulating in the bloodstream of the subject, and therefore the detection reagent may be used to detect or quantify the marker in a blood or serum sample from the subject. The terms “plasma” and “serum” are used interchangeably herein, unless otherwise noted. However, in some cases they are included in a single list of sample species to indicate that both are covered by the description or claim.

The term “tissue-specific polynucleotide,” as described herein generally refers to a polynucleotide that is predominantly expressed in a specific tissue. Often, methods, systems and kits disclosed herein utilize cell-free, tissue-specific polynucleotides. Cell-free, tissue-specific polynucleotides described herein are polynucleotides expressed at levels that can be quantified in a biological fluid upon damage of the tissue or organ in which they are expressed. In some cases, the presence of cell-free tissue-specific polynucleotides disclosed herein in a biological fluid is due to release of cell-free tissue-specific polynucleotides upon damage of the tissue or organ, and not due to a change in expression of the cell-free tissue-specific polynucleotides. Elevated levels of cell-free tissue-specific polynucleotides disclosed herein are indicative of damage to the corresponding tissue or organ. In some instances, cell-free polynucleotides disclosed herein are expressed/produced in several tissues, but at tissue-specific levels in at least one of those tissues. In these cases, the absolute or relative quantity of the cell-free tissue-specific polynucleotide is indicative of damage to a specific tissue or organ, or collection of tissues or organs. Alternatively or additionally, tissue-specific polynucleotides are nucleic acids with tissue-specific modifications. By way of non-limiting example, tissue-specific polynucleotides or markers disclosed herein include DNA molecules (e.g., a portion of a gene or non-coding region) with tissue-specific methylation patterns. In other words, the polynucleotides and markers may be expressed similarly in many tissues, or even ubiquitously throughout a subject, but the modifications are tissue-specific. Generally, tissue-specific polynucleotides or levels thereof disclosed herein are not specific to a disease. Generally, tissue-specific polynucleotides disclosed herein do not encode a protein implicated in a disease mechanism.

The term, “marker,” as used herein, encompasses a wide variety of biological molecules. Markers may also be referred to herein as disease markers or markers of disease. In some instances, the marker is for a condition associated with a plurality of diseases. For example, the marker may be for inflammation, which can be associated with cardiovascular disease, hepatitis and cancer. Markers, by way of non-limiting example, include peptides, hormones, lipids, vitamins, pathogens, cell fragments, metabolites and nucleic acids. In some instances, a marker is a cell-free nucleic acid. Generally, markers disclosed herein are not tissue-specific. However, in rare instances, the markers are tissue-specific. Markers disclosed herein may also be referred to as disease biomarkers. The disease biomarker is a biological molecule that is present or produced as a result of a disease, dysregulated as a result of a disease, mechanistically implicated in a disease, mutated or modified in a disease state, or any combination thereof. Markers may be produced by the subject. Markers may also be produced by other species. For instance, the marker may be a nucleic acid or protein made by a hepatitis virus or a Streptococcus bacterium. Methods identifying such markers may further comprise detecting/quantifying tissue-specific polynucleotides to determine which tissues are infected or affected by these pathogens, and optionally, to an extent that the tissue(s) are damaged. Markers of diseases disclosed herein generally do not circulate in individuals unaffected by the disease.

In general, the terms “cell free polynucleotide,” and “cell free nucleic acid,” used interchangeably herein, refers to a polynucleotide that can be isolated from a sample without extracting the polynucleotide from a cell. Cell free polynucleotides disclosed herein are typically polynucleotides that have been released or secreted from a damaged tissue or damaged organ. For example, damage to the tissue or organ may be due to a disease, injury or other condition that resulted in cytolysis, releasing the cell-free polynucleotide from cells of the damaged tissue into circulation. In some instances, a cell free polynucleotide disclosed herein is tissue-specific. In other instances, a cell free polynucleotide is not tissue-specific. In some instances, a cell free polynucleotide is present in a cell or in contact with a cell. In some instances, a cell free polynucleotide is in contact with an organelle, vesicle or exosome. In some instances, a cell free polynucleotide is cell-free, meaning the cell free polynucleotide is not in contact with a cell. Cell-free polynucleotides described herein are freely circulating, unless otherwise specified. In some instances, a cell free polynucleotide is freely circulating, that is the cell free polynucleotide is not in contact with any vesicle, organelle or cell. In some instances, a cell free polynucleotide is associated with a polynucleotide-binding protein (transferases, ribosomal proteins, etc.), but not any other molecules.

As used herein, the term ‘about’ a number refers to that number plus or minus 10% of that number. The term ‘about’ a range refers to that range minus 10% of its lowest value and plus 10% of its greatest value.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### Methods

As discussed in the foregoing and following description, methods disclosed herein are intended to non-invasively detect a tissue or organ in a subject that is under duress as well as determine which disease or condition is affecting the tissue or organ under duress. Some methods disclosed herein comprise determining a stage or progress of a disease or condition in a subject. Some methods disclosed herein comprise determining a response to a therapy used to treat a disease or condition in a subject. Some methods disclosed herein comprise determining if a particular tissue or organ in a subject is damaged, injured or infected. Some methods disclosed herein comprise determining if a particular tissue or organ in a subject is affected by a disease or condition. Some methods disclosed herein comprise detecting or quantifying a biological molecule disclosed herein. Some methods disclosed herein comprise detecting or quantifying a marker and/or tissue-specific polynucleotide disclosed herein.

Some methods disclosed herein comprise detecting, quantifying and/or analyzing at least one marker of a disease or condition in a sample of the subject. The methods may comprise detecting, quantifying, and/or analyzing at least one tissue-specific polynucleotide in a biological sample. The tissue-specific polynucleotide may be a tissue-specific, cell-free polynucleotides. The methods may further comprise comparing the quantity of the marker and/or the tissue-specific, cell-free polynucleotide to a reference level of the marker and a reference level of the tissue-specific polynucleotide, respectively. In some instances, comparison to a reference level is not required. For example, the presence of the marker and/or tissue-specific, cell-free polynucleotide may be sufficient to detect the disease or condition, or determine if the particular tissue is damaged, injured or infected by the disease or condition. In some aspects, the methods provide for the diagnosis or prognosis of the disease or condition, or assessing the progression thereof.

In some aspects, the present disclosure provides a method of determining whether a tissue has been damaged by a disease or condition. Often, the method comprises: (a) quantifying a level of or detecting at least one marker of a disease or condition in a first sample of a subject; (b) quantifying in a second sample of the subject a level of at least one tissue-specific polynucleotide, wherein the at least one tissue-specific polynucleotide is a cell-free polynucleotide, and further wherein the quantifying comprises at least one process selected from the group consisting of: reverse transcription, polynucleotide amplification, real-time PCR, sequencing, probe hybridization, microarray hybridization, and methylation-specific modification; (c) optionally comparing the level of the at least one marker to a corresponding reference level of the marker; (d) comparing the level of the at least one tissue-specific polynucleotide to a corresponding reference level of the tissue-specific polynucleotide; and (e) determining whether the tissue has been damaged by the disease or condition based on the comparing. The first sample and the second sample may be the same. The first sample and the second sample may be different. The first sample and the second sample may be obtained simultaneously. The first sample and the second sample may be obtained sequentially. By way of non-limiting example, the disease or condition may be selected from multiple sclerosis, hepatitis, liver steatosis conditions (NAFLD, NASH), heart disease, diabetes, cancer, a concurrent condition thereof, a complication thereof, a risk thereof, a stage thereof, and a response to a treatment thereof.

In another aspect, the disclosure provides a method of measuring a response to a pharmaceutical composition. In some embodiments, the method comprises: (a) quantifying a level of or detecting at least one marker of at least one condition in a first sample of a subject, wherein the first sample was obtained after an administration of the pharmaceutical composition; (b) quantifying in a second sample of a subject a level of at least one tissue-specific polynucleotide, wherein (i) the at least one tissue-specific polynucleotide is a cell-free polynucleotide specific to a tissue; and (ii) the second sample was obtained after the administration of the pharmaceutical composition; (c) optionally comparing the level of each of the at least one marker to a corresponding reference level of the marker, wherein the reference level of the marker is a level in a sample of the subject obtained prior to the administration of the pharmaceutical composition; (d) comparing the level of the at least one tissue-specific polynucleotide to a corresponding reference level of the tissue-specific polynucleotide, wherein the reference level of the tissue-specific polynucleotide is a level in a sample of the subject obtained prior to the administration of the pharmaceutical composition; and (e) determining whether the pharmaceutical composition has a therapeutic effect based on results of steps (c) and (d). The first sample and the second sample may be different. The first sample and the second sample may be obtained simultaneously. The first sample and the second sample may be obtained sequentially. By way of non-limiting example, the disease or condition may be selected from multiple sclerosis, hepatitis, liver steatosis (NAFLD, NASH), heart disease, diabetes, cancer, a concurrent condition thereof, a complication thereof, a risk thereof, a stage thereof, and a response to a treatment thereof.

Some methods disclosed herein comprise detecting a disease or condition in a subject and also detecting any tissues or organs that are under duress due to the disease or condition, wherein the methods comprise comparing levels of markers and/or cell-free polynucleotides in a biological sample to threshold levels of markers and/or cell-free polynucleotides. By way of example, detection of at least one inflammatory marker above a threshold level indicative of inflammation may be combined with a level of liver-specific RNA above a threshold level indicative of liver damage. Taken together, these results are used to determine that the liver is inflamed. By way of further example, a level of kidney-specific RNA that is not above a threshold level indicative of kidney damage may be used to determine, in combination with a high level of an inflammatory marker, that the subject is experiencing inflammation but not of the kidneys.

Some methods disclosed herein are used to detect liver damage. In some instances, the client is suspected of being affected by NAFLD or NASH. Treatments recommended or prescribed for NAFLD can be different than treatments recommended or prescribed for NASH. Thus, the methods disclosed herein may be used to differentiate NAFLD from NASH by detecting or quantifying markers (e.g., cell-free nucleic acids, inflammatory proteins, lipids, sterols, etc.) associated with each of these conditions. In some embodiments, the method comprises: (a) quantifying a level of at least one marker of a liver-associated disease or condition in a blood sample of a subject; (b) quantifying in the blood sample of the subject a level of at least one liver-specific polynucleotide, wherein the at least one tissue-specific polynucleotide is a cell-free polynucleotide, and optionally wherein the quantifying comprises methylation-specific modification; (c) comparing the level of the at least one marker to a corresponding reference level of the marker; (d) comparing the level of the at least one tissue-specific polynucleotide to a corresponding reference level of the tissue-specific polynucleotide; and (e) determining whether the tissue has been damaged by the liver-associated disease or condition based on the comparing. The liver-associated disease or condition may be non-alcoholic fatty liver disease or non-alcoholic steatohepatitis. The at least one liver-specific polynucleotide may be at least one nucleic acid or protein encoded by a gene selected from 1810014F10RIK, A1BG, ABCC2, ABCC6, ABCG5, ACOX2, ACSM2A, ADH1A, ADH1C, ADH6, AFM, AFP, AGXT, AHSG, AKR1C4, AKR1D1, ALB, ALDH1B1, ALDH4A1, ALDOB, AMBP, ANG, ANGPTL3, AOC3, APCS, APOA1, APOA2, APOA5, APOB, APOC1, APOC2, APOC3, APOC4, APOE, APOF, APOH, APOM, AQP9, ARID1A, ARSE, ASGR1, ASGR2, ASL, ATF5, C2, C2ORF72, C4A, C4BPA, C6, C8A, C8B, C8G, C9, CAPN5, CES1, CES2, CFHR1, CFHR2, CFHR3, CFHR4, CFHR5, CHD2, CIDEB, CPB2, CPN1, CRLF1, CRYAA, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F11, CYP4F12, CYP4F2, DAK, DCXR, DIO1, DUSP9, F10, F12, F2, F9, FAH, FCN2, FETUB, FGA, FGB, FGG, FMO3, FTCD, G6PC, GABBR1, GALK1, GAMT, GBA, GBP7, GCKR, GLYAT, GNMT, GPC3, GPT, GSTM1, HAAO, HAMP, HAO1, haptoglobin, HGD, HGFAC, HLF, HMGCS2, HP, HPD, HPN, HPR, HPX, HRG, HSD11B1, HSD17B6, IGF2, IGFALS, IGSF1, IL17RB, IL1RN, IQCE, ITIH1, ITIH2, ITIH2, ITIH3, ITIH4, JCLN, KHK, KLK13, LBP, LCAT, LECT2, LGALS4, LOC55908, LPA, MASP2, MAT1A, MBL2, MGMT, MST1, MSTP9, MUPCDH, NHLH2, NNMT, NR0B2, NR1I2, NSFL1C, OATP1B1, ORM1, ORM2, PCK1, PEMT, PGC, PKLR, PLG, PLGLB2, POLR2C, PON1, PON3, PROC, PXMP2, RBP4, RDH16, RELN, RET, RGN, RHBG, SAA4, SAA4, SARDH, SDS, SDSL, SEC14L2, SERPINA4, SERPINA5, SERPINA7, SERPINA10, SERPINA11, SERPINC1, SERPIND1, SERPINF2, SLC10A1, SLC22A1, SLC22A10, SLC22A7, SLC25A47, SLC27A5, SLC2A2, SLC38A3, SLC6A12, SLCO1B1, SPP2, SULT1A2, SULT2A1, TAT, TBX3, TCP10L, TF, TIM2, TMEM176B, TNNI2, TST, UGT2B15, UGT2B17 UPB1, UROC1, VTN, and WNT7A, and combinations thereof. Markers of liver disease include, but are not limited to cholesterol, triglycerides, insulin, glucose, leukocytes, free fatty acids, and inflammation-associated proteins (e.g. cytokines, chemokines).

In some aspects, the disclosure provide for methods of determining whether non-alcoholic fatty liver disease (NAFLD) is progressing or has progressed to non-alcoholic steatohepatitis (NASH) comprising: detecting at least one marker or quantifying a level of at least one marker of NAFLD and/or NASH in a first sample of a subject; quantifying in a second sample of the subject a level of a cell-free liver-specific polynucleotide of the subject; optionally comparing the level of the at least one marker to a corresponding reference level of the marker; optionally comparing the level of each of the at least one tissue-specific polynucleotide to a corresponding reference level of the tissue-specific polynucleotide; and determining whether non-alcoholic fatty liver disease has progressed to non-alcoholic steatohepatitis in the subject, or calculating a likelihood that such progression will occur. The at least one marker of non-alcoholic steatohepatitis may or may not be a marker of non-alcoholic fatty liver disease. The level of the at least one marker may be higher in a subject with non-alcoholic steatohepatitis than a subject with non-alcoholic fatty liver disease. The level of the at least one marker may be at least about 10% higher, at least about 20% higher, at least about 30% higher, at least about 40% higher, at least about 50% higher, at least about 60% higher, at least about 70% higher, at least about 80% higher, at least about 90% higher, or at least or about 100% higher than the level of the at least one marker in the subject with non-alcoholic fatty liver disease. The first sample and the second sample may be the same. The first sample and the second sample may be different. The first sample and the second sample may be obtained simultaneously. The first sample and the second sample may be obtained sequentially.

In some aspects, the disclosure provide for methods comprising: (a) quantifying a level of at least one marker of multiple sclerosis in a blood sample of a subject; (b) quantifying in the blood sample of the subject a level of at least one tissue-specific polynucleotide, wherein the at least one tissue-specific polynucleotide is a cell-free polynucleotide, and optionally wherein the quantifying comprises methylation-specific modification; (c) comparing the level of the at least one marker to a corresponding reference level of the marker; (d) comparing the level of the at least one tissue-specific polynucleotide to a corresponding reference level of the tissue-specific polynucleotide; and (e) determining whether neurological tissue has been damaged by multiple sclerosis based on the comparing. The neurological tissue may be selected from brain, neurons, and spinal cord. The at least one tissue-specific polynucleotide may be a nucleic acid or protein encoded by a gene selected from C3 proactivator, CRP, MBP, ORM, TNFRSF11B, CALCA, PLP1, VCAM-1, ICAM-1, ADAMTS4, BCAS1, CLDN11, CPM, CXCL16, EDGE, ELOVL7, ENPP6, ERBB3, EVI2A, FA2H, GAL3ST1, GJA12, GM98, GPR62, GSN, IL23A, MAG, MAL, MMP-9, MOBP, MOG, OPN, HGF, CCL4, EGF, CCL11, PLA2G4A, PLEKHH1, PLP1, PLXNB3, PRKCQ, SGK2, SRPK3, TMEM10, TNF-alpha, TRF, TSPAN2, and UGTA8, and combinations thereof. The at least one marker of multiple sclerosis, as well as other diseases and conditions, may be a non-peptide or non-polypeptide marker. The at least one marker of multiple sclerosis may be a marker of cellular immune system activation. A non-limiting example of a non-peptide or non-polypeptide marker and a marker of cellular immune system activation is neopterin.

In some aspects, the disclosure provide for methods comprising: (a) quantifying a level of at least one marker of a cardiovascular disease in a blood sample of a subject; (b) quantifying in the blood sample of the subject a level of at least one cardiovascular polynucleotide, wherein the at least one cardiovascular-specific polynucleotide is a cell-free polynucleotide, and optionally wherein the quantifying comprises methylation-specific modification; (c) comparing the level of the at least one marker to a corresponding reference level of the marker; (d) comparing the level of the at least one cardiovascular polynucleotide to a corresponding reference level of the cardiovascular polynucleotide; and (e) determining whether the cardiovascular system or a component thereof has been damaged by the cardiovascular disease or condition based on the comparing. The cardiovascular disease may also be referred to as a cardiovascular condition. The cardiovascular disease or condition may be atherosclerosis. The cardiovascular disease or condition may be coronary artery disease. The cardiovascular disease or condition may be atheroma. The cardiovascular disease or condition may be diabetic ischemic cardiomyopathy. The at least one cardiovascular polynucleotide may be encoded by a gene selected from TPH1, CNTN4, CASQ2, MYOCD, FHL5, ATRNL1, RPS6KA6, RYR2, NPR3, ACADL, PLCB4, ITLN1, FIBIN, SCRG1, MRAP2, CNN1, ANGPTL1, SLC22A3, PRUNE2, PLD5, NEGR1, SEMA3D, NPR1, PDZRN3, NPNT, PLN, MPP6, SBSPON, THRB, NEXN, TTLL7, PLIN2, CCR1, SELF, MMRN1, CD163, RGS1, NPL, CD180, C7, FPR3, ST8SIA2, ASB18, MYL3, PRSS42, LRRC10, TNNI3, MYL2, SMCO1, CCDC141, MYH7, RD3L, MYBPC3, TNNT2, SCN5A, GJA3, CSRP3, MT1HL1, MYOZ2, XIRP1, KLHL31, PLEKHA5, ANKRD46, PIK3R1, TPR, TRAK2, ALDH5A1, MGEA5, DUT, FAM134B, ARIH2, COL21A1, CBLB, SOBP, SLC16A7, ANP32E, PCMTD2, and EMCN, and combinations thereof.

Markers of cardiovascular disease include, but are not limited to cholesterol, triglycerides, free fatty acids, leukocytes, macrophages, foam cells, and inflammation-associated proteins such as interleukins, tissue-remodeling proteins, proteases, matrix metalloproteases, angiogenic factors, cytokines, and chemokines. At least one marker of atheroma may be encoded by a gene selected from, but not limited to, TPH1, CNTN4, CASQ2, MYOCD, FHL5, ATRNL1, RPS6KA6, NPR3, RYR2, ACADL, PLCB4, ITLN1, FIBIN, SCRG1, MRAP2, CNN1, ANGPTL1, SLC22A3, PRUNE2, PLDS, NEGR1, SEMA3D, NPR1, PDZRN3, NPNT, PLN, MPP6, SBSPON, THRB, NEXN, and TTLL7, and combinations thereof. At least one marker of diabetic ischemic cardiomyopathy may be encoded by a gene selected from, but not limited to, NPR3, PLEHA5, ANKRD46, PIK3R1, TPR, TRAK2, ALDH5A1, MGEA5, DUT, FAM134B, ARIH2, PIK3R1, COL21A1, CBLB, SOBP, SLC16A7, ANP32E, and PCMTD2, and combinations thereof.

In some aspects, the disclosure provide for methods comprising: (a) quantifying a level of at least one marker of a pancreas-associated disease or condition in a blood sample of a subject; (b) quantifying in the blood sample of the subject a level of at least one pancreas-specific polynucleotide, wherein the at least one pancreas-specific polynucleotide is a cell-free polynucleotide, and optionally, wherein the quantifying comprises methylation-specific modification; (c) comparing the level of the at least one marker to a corresponding reference level of the marker; (d) comparing the level of the at least one pancreas-specific polynucleotide to a corresponding reference level of the pancreas-specific polynucleotide; and (e) determining whether the heart has been damaged by the pancreas-associated disease or condition based on the comparing. The pancreas-associated disease or condition may be diabetes. The at least one marker of the pancreas-associated disease or condition may be insulin, glucose, or an inflammatory mediator (e.g., cytokine). The at least one pancreas-specific polynucleotide may be encoded by a gene selected from, but not limited to, REG1A, KLK1, GP2, REG1B, CPA2, CUZD1, PRSS3, CEL, AQP8, SERPINI2, CLPS, PLA2G1B, CPB1, PNLIPRP1, PLA2G1B, SPINK1, CTRB1, CTRC, ERP27, CELA2A, CPA1, C2orf83, CELA3B, GUCA1C, and G6PC2 and combinations thereof. Markers of pancreatic-associated disease include, but are not limited to, glucose, insulin, inflammation-associated proteins, and beta islet cell number.

In some aspects, the disclosure provide for methods comprising: (a) quantifying a level of at least one marker of a retina-associated disease or condition in a blood sample of a subject; (b) quantifying in the blood sample of the subject a level of at least one retina-specific polynucleotide, wherein the at least one retina-specific polynucleotide is a cell-free polynucleotide, and optionally wherein the quantifying comprises methylation-specific modification; (c) comparing the level of the at least one marker to a corresponding reference level of the marker; (d) comparing the level of the at least one retina-specific polynucleotide to a corresponding reference level of the retina-specific polynucleotide; and (e) determining whether the retina has been damaged by the retina-associated disease or condition based on the comparing. The at least one retina-specific polynucleotide may be encoded by a gene selected from, but not limited to, RBP3, OPTC, RHO, RPE65, RLBP1, GNAT1, OTX2, RCVRN, RGR, PPEF2, PDC, SIX3, PDE6G, CRYBA1, RGR, ARR3, NRL, PDE6A, SAG, LRAT, AIPL1, GUCA1A, GNGT1, and GRIM, and combinations thereof. The retina-associated disease or condition may be diabetic retinopathy. The at least one marker of diabetic retinopathy or retina-specific polynucleotides may be encoded by a gene selected from, but not limited to, RBP3, OPTC, RHO, RPE65, RLBP1, GNAT1, OTX2, RCVRN, RGR, PPEF2, PDC, SIX3, PDE6G, CRYBA1, RGR, ARR3, IMPG1, NRL, PDE6A, SAG, LRAT, AIPL1, GUCA1A, GNGT1, PMEL, TYRP1, BEST1, RGR, MLAVA, TYR, BCO1, TSPAN10, SLC39A12, SLC45A2, SLC16A8, DCT, SGRP5, MYOC, EDN3, COL9A1, TRPM3, MYOC, and GRIM, and combinations thereof. The at least one marker of diabetic retinopathy or the at least one retina-specific polynucleotide may be encoded by a gene selected from, but not limited to, BEST1, RGR, and PMEL, and combinations thereof.

In some aspects, the disclosure provide for methods comprising: (a) quantifying a level of at least one marker of a kidney-associated disease or condition in a blood sample of a subject; (b) quantifying in the blood sample of the subject a level of at least one kidney-specific polynucleotide, wherein the at least one kidney-specific polynucleotide is a cell-free polynucleotide, and optionally wherein the quantifying comprises methylation-specific modification; (c) comparing the level of the at least one marker to a corresponding reference level of the marker; (d) comparing the level of the at least one kidney-specific polynucleotide to a corresponding reference level of the kidney-specific polynucleotide; and (e) determining whether the kidney has been damaged by the kidney-associated disease or condition based on the comparing. The kidney-associated disease or condition may be diabetic nephropathy. The kidney-specific polynucleotide may be encoded by a gene selected from, but not limited to, SLC12A3, SLC12A1, SLC22A2, HAVCR1, SLC34A1, DNMT3L, KAAG1, ATP6V0D2, SLC22A8, ATPV1G3, BSND, FCAMR, TMEM174, SLC6A18, AQP2, SLC22A11, SLC22A13, SLC22A12, TMEM207, MCCD1, UMOD, NPHS2, SLC4A9, PAX2, MIOX, CDH16, UGT1A9, 00001T8, CASR, CYP24A1, DPEP1, DUSP9, FMO1, HNF1A, KHK, LGALS2, NPHS1, PAPPA2, PTH2R, SLC12A1, SLC12A3, SLC6A13, TDGF1, UMOD, and XPNPEP2, and combinations thereof. The at least one markers of diabetic nephropathy may be encoded by a gene selected from, but not limited to, CASR, CYP24A1, DPEP1, DUSP9, FMO1, HNF1A, KHK, LGALS2, NPHS1, PAPPA2, PTH2R, SLC12A1, SLC12A3, SLC6A13, TDGF1, UMOD, and XPNPEP2, and combinations thereof. The at least one marker of diabetic nephropathy may be encoded by a gene selected from, but not limited to, CYP24A1, NPHS1, SLC12A1, SLC12A3, and UMOD, and combinations thereof.

In some aspects, the disclosure provides for methods of monitoring a human subject with a chronic condition for a presence of at least one complication of at least one tissue. In some aspects, the disclosure provide for methods of monitoring a human subject with a chronic condition for an increased risk of at least one complication of at least one tissue.

In some aspects, the disclosure provide for methods of monitoring a human subject with a chronic metabolic condition for a presence of at least one complication of at least one tissue. In some aspects, the disclosure provide for methods of monitoring a human subject with a chronic metabolic condition for an increased risk of at least one complication of at least one tissue.

In some aspects, the disclosure provide for methods of assessing a sample from a human subject with a chronic metabolic condition for a presence of at least one complication of at least one tissue. In some aspects, the disclosure provide for methods of assessing a sample from a human subject with a chronic metabolic condition for an increased risk of at least one complication of at least one tissue.

Some methods comprise monitoring the human subject for a complication in any one of at least three tissues. Some methods comprise monitoring the human subject for an increased risk of a complication in any one of at least three tissues.

Some methods comprise the steps of: obtaining a biological fluid from the subject; measuring a marker level in the biological fluid, wherein the marker is selected from a cholesterol, a lipid, insulin, an inflammatory mediator, a lipid mediator, an insulin mediator and a cholesterol mediator; and quantifying ribonucleic acids (RNA) in the biological fluid from liver, cardiovascular tissue, and kidney. In some cases, a threshold marker level and a threshold quantity of the RNA indicates the presence or increased risk of the complication in at least one of the liver, cardiovascular tissue and kidney.

As used herein, the term “chronic condition” is a condition that the subject has experienced for at least about six months. In some instances, a chronic condition is a condition that the subject has experienced for at least about one year. In some instances, a chronic condition is a condition that the subject has experienced for at least about six months to at least about one year. In some instances, a chronic condition is a condition that the subject has experienced for at least about six months to at least about two years. In some instances, the chronic condition is a chronic metabolic condition. In some instances, the chronic condition is a neurodegenerative condition. In some instances, the chronic condition is cancer.

As used herein, the term “complication” includes a condition that is acute, a condition that is life-threatening, a condition that requires immediate intervention, a condition that warrants immediate attention, a condition of which immediate attention or intervention would prevent a life-threatening incident, and combinations thereof. Non-limiting examples of complications are renal ischemia, renal failure, liver failure, liver cirrhosis, liver fibrosis, non-alcoholic steatohepatitis, viral hepatitis, arterial thrombosis, arterial occlusion, valvular heart disease, atherosclerotic plaques, aneurysm, peripheral artery disease, blood clot, pericarditis, and cardiomyopathy.

In some instances, an increased risk of at least one complication is a substantially greater risk in the subject relative to a risk of the at least one complication in a subject that does not have a chronic metabolic condition. In some instances, an increased risk of at least one complication is a substantially greater risk in a first subject that has the chronic metabolic condition relative to a risk of the at least one complication in a second subject that does not have the chronic metabolic condition.

Often, methods disclosed herein comprise detecting or quantifying an amount of a marker of a disease or condition disclosed herein in to determine that the subject is affected by a respective disease or condition or that the subject is at a risk of being affected by a respective disease or condition. In some instances, detecting or quantifying at least 1 copy/ml of the marker is sufficient to determine that the subject is affected by, or at risk of being affected by, a respective disease or condition. In some instances, detecting or quantifying at least 5 copies/ml of the marker is sufficient to determine that the subject is affected by, or at risk of being affected by, a respective disease or condition. In some instances, detecting or quantifying at least 10 copies/ml of the marker is sufficient to determine that the subject is affected by, or at risk of being affected by, a respective disease or condition. In some instances, detecting or quantifying at least 15 copies/ml of the marker is sufficient to determine that the subject is affected by, or at risk of being affected by, a respective disease or condition. In some instances, detecting or quantifying at least 20 copies/ml of the marker is sufficient to determine that the subject is affected by, or at risk of being affected by, a respective disease or condition. In some instances, detecting or quantifying at least 25 copies/ml of the marker is sufficient to determine that the subject is affected by, or at risk of being affected by, a respective disease or condition. In some instances, detecting or quantifying at least 30 copies/ml of the marker is sufficient to determine that the subject is affected by, or at risk of being affected by, a respective disease or condition. In some instances, detecting or quantifying at least 40 copies/ml of the marker is sufficient to determine that the subject is affected by, or at risk of being affected by, a respective disease or condition. In some instances, detecting or quantifying at least 50 copies/ml of the marker is sufficient to determine that the subject is affected by, or at risk of being affected by, a respective disease or condition. In some instances, detecting or quantifying at least 100 copies/ml of the marker is sufficient to determine that the subject is affected by, or at risk of being affected by, a respective disease or condition.

Often, methods disclosed herein comprise detecting or quantifying an amount of a tissue-specific polynucleotide disclosed herein in to determine that a respective tissue is being affected by a disease or condition. In some instances, methods comprise detecting or quantifying at least 1 copy/ml of the tissue-specific polynucleotide. In some instances, methods comprise detecting or quantifying at least 5 copies/ml of the tissue-specific polynucleotide. In some instances, methods comprise detecting or quantifying at least 10 copies/ml of the tissue-specific polynucleotide. In some instances, methods comprise detecting or quantifying at least 15 copies/ml of the tissue-specific polynucleotide. In some instances, methods comprise detecting or quantifying at least 20 copies/ml of the tissue-specific polynucleotide. In some instances, methods comprise detecting or quantifying at least 25 copies/ml of the tissue-specific polynucleotide. In some instances, methods comprise detecting or quantifying at least 30 copies/ml of the tissue-specific polynucleotide. In some instances, methods comprise detecting or quantifying at least 35 copies/ml of the tissue-specific polynucleotide. In some instances, methods comprise detecting or quantifying at least 40 copies/ml of the tissue-specific polynucleotide. In some instances, methods comprise detecting or quantifying at least 45 copies/ml of the tissue-specific polynucleotide. In some instances, methods comprise detecting or quantifying at least 50 copies/ml of the tissue-specific polynucleotide. In some instances, methods comprise detecting or quantifying at least 100 copies/ml of the tissue-specific polynucleotide.

Some methods disclosed herein comprise detecting or quantifying at least a certain amount of a marker or tissue-specific polynucleotide in order to determine that a disease or condition is affecting a respective tissue. In some cases, the amount of the marker, wherein the marker is a polynucleotide, or tissue-specific polynucleotide is at least about 1 copy/mL, at least about 10 copies/mL, at least about 20 copies/mL, at least about 30 copies/mL, at least about 40 copies/mL, or at least about 50 copies/mL, at least about 80 copies/cell, at least about 100 copies/cell, at least about 120 copies/cell, at least about 150 copies/cell, or at least about 200 copies/cell. In some cases, the amount of the marker, wherein the marker is a protein, lipid, or other non-polynucleotide biological molecule, is at least about 5 pg/mL, at least about 10 pg/mL, at least about 20 pg/mL, at least about 30 pg/mL, at least about 50 pg/mL, at least about 60 pg/mL, at least about 80 pg/mL, at least about 100 pg/mL, at least about 150 pg/mL, at least about 200 pg/mL, or at least about 500 pg/mL.

### Isolating, Quantifying and Detecting

As discussed in the foregoing and following description, methods and systems disclosed herein are intended to non-invasively detect a tissue or organ in a subject that is under duress as well as determine which disease or condition is affecting the tissue or organ under duress by detecting, quantifying, or otherwise analyzing at least one marker and at least one tissue-specific polynucleotide disclosed herein. In some cases, the at least one marker comprises a polynucleotide (e.g. cell-free polynucleotide) or a polypeptide. Some methods comprise detecting the polynucleotide or polypeptide by contacting the polynucleotide or polypeptide with at least one probes. In some cases, the at least one probe is only be capable of binding to a wildtype version of the polynucleotide or polypeptide. In some cases, the at least one probe is only be capable of binding to a mutant version of the polynucleotide or polypeptide. In some cases, wherein the marker is a polynucleotide, detection comprises sequencing.

Some methods disclosed herein comprise isolating at least one marker and/or at least one tissue-specific polynucleotide. In some cases, the at least one marker and/or at least one tissue-specific polynucleotide comprise a cell-free polynucleotide. In some cases, isolating the cell-free polynucleotide comprises fractionating the sample from the subject. Some methods comprise removing intact cells from the sample. For example, some methods comprise centrifuging a blood sample and collecting the supernatant that is serum or plasma, or filtering the sample to remove cells. In some embodiments, cell-free polynucleotides are analyzed without fractionating the sample from the subject. For example, urine, cerebrospinal fluid or other fluids that contain little to no cells may not require fractionating. Some methods comprise sufficiently purifying the cell-free polynucleotides in order to detect/quantify/analyze the cell-free polynucleotides. Various reagents, methods and kits can be used to purify the cell-free polynucleotides. Reagents are known in the art and include, but are not limited to, Trizol, phenol-chloroform, glycogen, sodium iodide, and guanidine resin. Kits include, but are not limited to, Thermo Fisher ChargeSwitch® Serum Kit, Qiagen RNeasy Kit, ZR serum DNA kit, Puregene DNA purification system, QIAamp DNA Blood Midi kit, QIAamp Circulating Nucleic Acid Kit, and QIAamp DNA Mini kit.

Some methods disclosed herein comprise enriching a sample for cell-free polynucleotides. For example, a sample of interest may contain RNA/DNA from bacteria. Some methods comprise exomal capture, thereby eliminating unwanted sequences and enriching the sample for polynucleotides of interest. In some cases, exomal capture comprises array-based capture or in-solution capture, fragments of DNA corresponding to RNAs of interest tethered to a surface or beads, respectively. Some methods also comprise filtering or removing other biological molecules or cells from the sample, such as proteins or platelets. In some instances, enriching the sample for cell-free polynucleotides includes preventing blood cell RNA contamination of a plasma sample. In some instances, using tubes free of EDTA prevents or reduces the presence of blood cell RNA in a plasma/serum sample.

Generally, methods disclosed herein comprise detecting or quantifying at least one marker and/or at least one tissue-specific polynucleotide. In some instances, quantifying and/or detecting the at least one marker and/or at least one tissue-specific polynucleotide comprises amplifying the at least one marker and/or at least one tissue-specific polynucleotide. In some cases involving cell-free RNA, quantifying and/or detecting the at least one marker and/or at least one tissue-specific polynucleotide comprises reverse transcribing the cell-free RNA. Any of a variety of processes can be employed to detect/quantify the marker or tissue-specific polynucleotide in a sample. In some cases involving cell-free, tissue-specific RNAs, RNA is isolated from a sample and reverse transcribed to produce cDNA prior to further manipulation, such as amplification and/or sequencing. In some embodiments, amplification is initiated at the 3′ end as well as randomly throughout the whole transcriptome in the sample to allow for amplification of both mRNA and non-polyadenylated transcripts. Suitable kits for amplifying cDNA include, for example, the Ovation® RNA-Seq System. Tissue-specific RNAs can be identified and quantified by a variety of techniques known in the art, such as array hybridization, quantitative PCR, and sequencing.

Some methods disclosed herein comprise quantifying at least one marker and/or at least one tissue-specific polynucleotide described herein. In some cases, quantifying is useful for determining the severity of a condition. For example, some methods comprise comparing a quantity of marker and/or tissue-specific polynucleotide to a quantity of marker and/or tissue-specific polynucleotide in a sample from a subject in an early state of the condition or an advanced state of the condition. Some methods comprise determining if a therapy is appropriate based on the severity of the condition or state of the condition. Some methods comprise determining an appropriate therapeutic dose based on the severity of the condition or state of the condition. Quantifying may be useful in monitoring and modulating a therapy for the condition. For example, some methods comprise quantifying the marker and/or tissue-specific polynucleotide in a first sample at a first time in the subject and quantifying the marker and/or tissue-specific polynucleotide in a second sample at a second time, wherein the subject was subjected to a therapy between the first time and the second time. Some methods comprise maintaining the therapy or changing the therapy (e.g., type, dose) based on information that resulted from the quantifying. Some methods comprise quantifying the marker and/or tissue-specific polynucleotide in additional samples at additional times, in between which the therapy is modulated.

Some methods of quantifying nucleic acids disclosed herein comprise sequencing at least one nucleic acid. Sequencing may be targeted sequencing. In some cases, targeted sequencing comprises specifically amplifying a select marker or a select tissue-specific polynucleotide disclosed herein and sequencing the amplification products. In some cases, targeted sequencing comprises specifically amplifying a subset of selected markers or a subset of select tissue-specific polynucleotide disclosed herein and sequencing the amplification products. Alternatively, some methods comprising targeting sequencing do not comprise amplifying the markers or tissue-specific polynucleotides. Some methods comprise untargeted sequencing. In some instances, untargeted sequencing comprises sequencing the amplification products, wherein a portion of the cell-free nucleic acids are not markers or tissue-specific polynucleotides. In some instances, untargeted sequencing comprises amplifying cell-free nucleic acids in a sample from the subject and sequencing the amplification products, wherein a portion of the cell-free nucleic acids are not markers or tissue-specific polynucleotides. In some instances, untargeted sequencing comprises amplifying cell-free nucleic acids comprising a marker or tissue-specific polynucleotide described herein. Sequencing may provide a number of reads that corresponds to a relative quantity of the marker or tissue-specific polynucleotide. In some instances, sequencing provides a number of reads that corresponds to an absolute quantity of the marker or tissue-specific polynucleotide. In some embodiments, the amplified cDNA is sequenced by whole transcriptome shotgun sequencing (also referred to as “RNA-Seq”). Whole transcriptome shotgun sequencing (RNA-Seq) can be accomplished using a variety of next-generation sequencing platforms such as the Illumina Genome Analyzer platform, ABI Solid Sequencing platform, or Life Science's 454 Sequencing platform. In some instances, identification of specific targets is performed by microarray, such as a peptide array or oligonucleotide array, in which an array of addressable binding elements specifically bind to corresponding targets, and a signal proportional to the degree of binding is used to determine quantity of the target in the sample. In some cases, sequencing is a preferable method of quantifying. In some instances, sequencing allows for parallel interrogation of thousands of genes without amplicon interference. In some instances, quantifying by sequencing is preferable to quantifying by Q-PCR. In some instances, there are so many control genes required to accurately quantify gene expression by Q-PCR, that quantifying with Q-PCR is inefficient. In other instances, sequencing efficiency and accurate quantification by sequencing is not be affected by the number of (control) genes analyzed. For at least the foregoing reasons, sequencing is particularly useful for some methods disclosed herein, wherein the health status of multiple organs (e.g., heart, kidney and liver) is assessed.

Some methods of quantifying a nucleic acid disclosed herein comprise quantitative PCR (q-PCR). In some instances, Q-PCR comprises a reverse transcription reaction of cell-free RNAs described herein to produce corresponding cDNAs. In some instances, cell-free RNA comprises a marker, a tissue-specific polynucleotide, and a cell-free RNA that is neither a marker nor a tissue specific polynucleotide. Some cell-free RNA comprises a marker described herein, a tissue-specific polynucleotide described herein, and a cell-free RNA that is neither a marker nor a tissue specific polynucleotide described herein. In some cases, Q-PCR comprises contacting the cDNAs that correspond to a marker, a tissue-specific polynucleotide, or a housekeeping gene (e.g., ACTB, ALB, GAPDH) with PCR primers specific to the marker, tissue-specific polynucleotide or housekeeping gene.

Some methods disclosed herein comprise quantifying a blood cell-specific polynucleotide. Methods comprising Q-PCR disclosed herein may comprise contacting cDNA with primers corresponding to a blood cell-specific polynucleotide. Some blood cell-specific polynucleotides disclosed herein are nucleic acids that are predominantly expressed or even exclusively expressed by one or more types of blood cells. Types of blood cells can be generally categorized as white blood cells (also referred to as leukocytes), red blood cells (also referred to as erythrocytes), and platelets. In some instances, the blood cell-specific polynucleotide is used as a control in methods comprising quantifying tissue-specific polynucleotides and disease markers disclosed herein. In some cases, absence of an amplification product with primers corresponding to a blood cell-specific polynucleotide may be used to confirm the method is detecting cell-free RNAs in a blood, plasma or serum sample and not RNA expressed in blood cells. By way of non-limiting example, blood-cell specific polynucleotides include polynucleotides expressed in white blood cells, platelets or red blood cells, and combinations thereof. White blood cells, include, but are not limited to lymphocytes, T-cells, B cells, dendritic cells, granulocytes, monocytes, and macrophages. By way of non-limiting example, the blood-specific polynucleotide may be encoded by a gene selected from CD4, TMSB4X, MPO, SOX6, HBA1, HBA2, HBB, DEFA4, GP1BA, CD19, AHSP, and ALAS2. The blood cell-specific polynucleotide may be encoded by CD4 and predominantly expressed by white blood cells. The blood cell-specific polynucleotide may be encoded by TMSB4X and expressed by multiple blood cell types (whole blood). The blood cell-specific polynucleotide may be encoded by MPO and predominantly expressed by neutrophil granulocytes. The blood cell-specific polynucleotide may be encoded by DEFA4 and predominantly expressed by neutrophils. The blood cell-specific polynucleotide may be encoded by GP1BA and predominantly expressed by platelets. The blood cell-specific polynucleotide may be encoded by CD19 and predominantly expressed by B cells. The blood cell-specific polynucleotide may be encoded by ALAS2, SOX6, HBA1, HBA2 or HBB and predominantly expressed by erythrocytes.

In some cases, Q-PCR is a preferable method of quantifying. Q-PCR may be a more sensitive method and therefore more accurately quantify RNA present at very low levels. In some instances, quantifying by Q-PCR is preferable to quantifying by sequencing. In some instances, sequencing requires more complex preparation of RNA samples and requires depletion or enrichment of nucleic acids in order to provide accurate quantification.

Often, methods disclosed herein comprise detecting or quantifying a combination of markers or a combination of tissue-specific polynucleotides. In some cases, a more conclusory diagnosis or assessment of the subject can be performed if multiple tissue-specific polynucleotides are detected. In some cases, the presence of each of the tissue-specific polynucleotides in a blood sample of the subject would not be indicative of damage to the tissue or origin of interest. However, their presence may collectively indicate damage to the tissue or origin of interest. Similarly, a more conclusory diagnosis or assessment of the subject can be performed if multiple markers are detected. In some cases, the presence of each of the markers in a blood sample of the subject would not be indicative of damage to the tissue or origin of interest. However, their presence may collectively indicate the condition in the tissue or origin of interest. The methods may comprise detecting or quantifying about 2, about 3, about 4, about 5, about 6, about 7, about 8, about 9 or about 10 tissue-specific polynucleotides. The methods may comprise detecting or quantifying about 2, about 3, about 4, about 5, about 6, about 7, about 8, about 9 or about 10 markers. Two or more of the markers may be known to interact in a common genetic pathway or common molecular signaling pathway. The common molecular signaling pathway may be a network of several proteins interacting to enact a cellular function, such as, by way of non-limiting example, an inflammatory response, apoptosis, cholesterol uptake, etc.

Similarly, in the case of cell-free DNAs, some methods disclosed herein employ tissue-specific modifications of DNA or chromatin to identify the tissue-specific polynucleotide in the sample. For example, a tissue-specific cell-free DNA may comprise a tissue-specific methylation pattern. A tissue-specific cell-free DNA may be complexed with a protein that is indicative of a specific tissue of origin (e.g., a transcription factor known to transcribe the gene in a particular tissue). Cell-free or circulating chromatin or chromatin fragments may have tissue-specific histone modifications (e.g., methylation, acetylation, and phosphorylation). In some of these cases, a method such as chromatin immunoprecipitation may be suitable for detecting/quantifying the tissue-specific polynucleotide. Cell-free tissue-specific DNA may be single-stranded or double-stranded DNA.

Some methods disclosed herein comprise use of a variety of methods of detecting the methylation pattern. Typically, the DNA will be subjected to a chemical conversion process that selectively modified either methylated or unmethylated nucleotides. For example, the DNA may be treated with bisulfite, which converts cytosine residues to uracil (which are converted to thymidine following PCR), but leaves 5-methylcytosine residues unaffected. Thus, bisulfite treatment introduces specific changes in the DNA sequence that depend on the methylation status of individual cytosine residues (“methylation-specific modification”), yielding single-nucleotide resolution information about the methylation status of a segment of DNA. Various analyses can be performed on the altered sequence to retrieve this information.

Some methods disclosed herein comprise subjecting DNA to oxidizing or reducing conditions prior to bisulfite treatment, so as to identify patterns of other epigenetic marks. For example, an oxidative bisulfite reaction can be performed. 5-methylcytosine and 5-hydroxymethylcytosine both read as a C in bisulfite sequencing. An oxidative bisulfite reaction allows for the discrimination between 5-methylcytosine and 5-hydroxymethylcytosine at single base resolution. Typically, the method employs a specific chemical oxidation of 5-hydroxymethylcytosine to 5-formylcytosine, which subsequently converts to uracil during bisulfite treatment. The only base that then reads as a C is 5-methylcytosine, giving a map of the true methylation status in the DNA sample. Levels of 5-hydroxymethylcytosine can also be quantified by measuring the difference between bisulfite and oxidative bisulfite sequencing. DNA may also be subjected to reducing conditions prior to bisulfite treatment. Reduction converts 5-formylcytosine residues in the sample nucleotide sequence into 5-hydroxymethylcytosine. As noted above, 5-formylcytosine converts to uracil upon bisulfite treatment, but 5-hydroxymethylcytosine does not. By comparing a first portion of a sample subjected to reductive bisulfite treatment to a second portion of a sample subjected to bisulfite treatment alone, locations of 5-formylcytosine marks can be identified.

As an alternative to inducing sequence changes based on methylation, methods disclosed herein may comprise inferring methylation status may by isolating or enriching polynucleotides comprising methylation, and identifying the methylated polynucleotides based on their sequences (e.g. by sequencing or probe hybridization). One process for enriching methylated sequences comprises modifying bases in a methylation-specific fashion, enriching for polynucleotides comprising the modification (e.g. by purification), optionally amplifying the enriched polynucleotides, and then identifying the polynucleotides. For example, 5-hydroxymethyl-modified cytosines (5hmC) may be selectively glycosylated in the presence of a UDP-glucose molecules and a beta-glucosyltransferase. The UDP-glucose molecules may comprise a label, such that the label becomes conjugated to the 5hmC-containing polynucleotide upon reaction with the UDP-glucose. The label can be a member of a binding pair (e.g. steptavidin/biotin or antigen/antibody), which allows isolation of modified fragments upon binding to the corresponding member of the binding pair. Isolated polynucleotides may be further enriched, such as in an amplification reaction (e.g. PCR), prior to identification.

Presence and optionally quantity (relative or absolute) of a polynucleotide, as well as changes in sequence resulting from bisulfate treatment, can be detected using any suitable sequence detection method disclosed herein. Examples include, but are not limited to, probe hybridization, primer-directed amplification, and sequencing. Polynucleotides may be sequenced using any convenient low or high throughput sequencing technique or platform, including Sanger sequencing, Solexa-Illumina sequencing, Ligation-based sequencing (SOLiD), pyrosequencing; strobe sequencing (SMR); and semiconductor array sequencing (Ion Torrent). The Illumina or Solexa sequencing is based on reversible dye-terminators. DNA molecules are typically attached to primers on a slide and amplified so that local clonal colonies are formed. Subsequently, one type of nucleotide at a time may be added, and non-incorporated nucleotides are washed away. Subsequently, images of the fluorescently labeled nucleotides may be taken and the dye is chemically removed from the DNA, allowing a next cycle. The Applied Biosystems' SOLiD technology employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. Another example of an envisaged sequencing method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of suitable sequencing techniques are sequencing by hybridization, sequencing by use of nanopores, microscopy-based sequencing techniques, microfluidic Sanger sequencing, or microchip-based sequencing methods. High-throughput sequencing platforms permit generation of multiple different sequencing reads in a single reaction vessel, such as 10³, 10⁴, 10⁵, 10⁶, 10⁷ or more.

Some methods, systems and kits disclosed herein provide for quantifying a tissue's relative contribution to a cell-free transcriptome of a biological sample. In some instances quantifying a tissue's relative contribution to a cell-free transcriptome comprises quantifying total RNA in the sample. In some instances quantifying a tissue's relative contribution to a cell-free transcriptome comprises quantifying total nucleic acids in the sample. In some instances, the relative contribution of the tissue is compared to that of a control cell-free transcriptome in a control sample. If the relative contribution of the tissue is similar to that of a control cell-free transcriptome, the tissue is considered to have a similar health status as that of a control tissue contributing to the control cell-free transcriptome. If the relative contribution of the tissue is different from that of a control cell-free transcriptome, the tissue is considered to have a different health status than that of a control tissue contributing to the control cell-free transcriptome. See, e.g., FIG. 2.

In some cases the control cell-free transcriptome is representative of a healthy individual or a healthy population with the control tissue being healthy, disease-free, and damage-free. For healthy, normal subjects, the relative contributions of circulating RNA from different tissue types are usually stable relative to subjects with conditions or diseases. Thus, relative proportions of the cell-free transcriptome corresponding to various tissues can serve as reference levels. In some cases the control cell-free transcriptome is representative of an individual or a population with a disease or condition with the control tissue being affected by a disease or condition.

In some instances quantifying a tissue's relative contribution to a cell-free transcriptome comprises quantifying a first tissue's relative contribution to a cell-free transcriptome and a second tissue's relative contribution to the cell-free transcriptome. In some instances, the relative contributions of the first and second tissues are compared to those of a control cell-free transcriptome. If the relative contributions of the first and second tissues are similar to those of a control cell-free transcriptome, the tissues are considered to have a similar health status as those of control tissues contributing to the control cell-free transcriptome. If the relative contributions of the first and second tissues are different from those of a control cell-free transcriptome, the tissues are considered to have a different health status than those of control tissues contributing to the control cell-free transcriptome.

Some methods and systems disclosed herein provide for deconvolution of a cell-free transcriptome to determine the relative contribution of a tissue type towards the cell-free RNA transcriptome. In some instances, the following steps are employed to determine the relative RNA contributions of certain tissues in a sample, First, a panel of tissue-specific transcripts is identified. Second, total RNA in plasma from a sample is determined using methods known in the art. Third, the total RNA is assessed against the panel of tissue-specific transcripts, and the total RNA is considered a summation these different tissue-specific transcripts. Quadratic programming can be used as a constrained optimization method to deduce the relative optimal contributions of different organs/tissues towards the cell-free transcriptome of the sample. In certain embodiments, quadratic programming is used as a constrained optimization method to deduce relative optimal contributions of different organs/tissues towards the cell-free transcriptome in a sample. Quadratic programming is known in the art and described in detail in Goldfarb and A. Idnani (1982). Dual and Primal-Dual Methods for Solving Strictly Convex Quadratic Programs. In J. P. Hennart (ed.), Numerical Analysis, Springer-Verlag, Berlin, Pages 226-239, and D. Goldfarb and A, Idnani (1983), A numerically stable dual method for solving strictly convex quadratic programs. Mathematical Programming, 27, 1-33.

In some cases, the methods comprise normalizing cell-free transcript values. This involves resealing cell-free transcript values to housekeeping gene transcript values. Next, the sample's total RNA is assessed against the panel of tissue-specific genes using quadratic programming in order to determine the tissue-specific relative contributions to the sample's cell-free transcriptome. The following constraints are employed to obtain the estimated relative contributions during the quadratic programming analysis: a) the RNA contributions of different tissues are greater than or equal to zero, and b) the sum of all contributions to the cell-free transcriptome equals one.

Some methods, systems and kits disclosed herein provide for determining the relative contribution of a tissue to determine a reference level for the tissue. That is, a certain population of subjects (e.g., diseased, normal, cancerous) can be subject to the deconvolution process to obtain reference levels of tissue-specific gene expression for a reference population, also referred to as control population. When relative tissue contributions are considered individually, quantification of each of these tissue-specific transcripts can be used as a measure of a reference apoptotic rate, cell turnover rate, senescence rate, nucleic acid release rate or secretion rate of that particular tissue for that particular population. For example, blood from one or more healthy, normal individuals can be analyzed to determine the relative RNA contribution of tissues to the cell-free RNA transcriptome for healthy, normal individuals. Each relative RNA contribution of tissue that makes up the normal RNA transcriptome is a reference level for that tissue.

Some methods disclosed herein comprise deducing relative contributions of different tissue types. A quantified panel of tissue specific transcripts can be considered as a summation of the contributions from the various tissues. Relative contributions of different tissue types may be obtained by inserting observed transcript levels in a sample tissue and a reference tissue into the following equation to determine πᵢ for each tissue, which will correspond to the fractional contribution the sample tissue(s) to the cell-free transcriptome.

$${Y}_{i} = {\sum }_{j} {π}_{i} {X}_{ij} + ε$$

where Y is the observed transcript quantity in a sample for gene i, X is the known transcript quantity for gene i in a reference tissue j and ε the normally distributed error. Additional physical constraints include:

1. Summation of all fraction contributing to the observed quantification is 1, given by the condition: Σπᵢ=1

2. All the contribution from each tissue type has to greater than or equal zero. There is no physical meaning to having a negative contribution. This is given by πᵢ≥0, since Σ is defined as the fractional contribution of each tissue types.

Consequently to obtain the optimal fractional contribution of each tissue type, the least-square error is minimized. The above equations are then solved using quadratic programming in R to obtain the optimal relative contributions of the tissue types towards the maternal cell free RNA transcripts. In the workflow, the quantity of RNA transcripts are given relative to the housekeeping genes in terms of Ct values obtained from qPCR. Therefore, the Ct value can be considered as a proxy of the measured transcript quantity. An increase in Ct value of one is similar to a two-fold change in transcript quantity, i.e. 2 raised to the power of 1. The process beings with normalizing all of the data in CT relative to the housekeeping gene, and is followed by quadratic programming.

### Treating, Monitoring, and Testing

As discussed in the foregoing and following description, methods, systems and kits disclosed herein are intended to non-invasively detect a tissue or organ in a subject that is under duress as well as determine which disease or condition is affecting the tissue or organ under duress. In some instances, the methods, systems and kits provide for treating a subject for a disease or condition. Some methods disclosed herein comprise selecting a method or therapy for treating a subject for a disease or condition. Some kits and systems disclosed herein provide for selecting a method or therapy for treating a subject for a disease or condition. Some methods disclosed herein comprise monitoring a disease or condition in a subject, or administering a test for a disease or condition. Some kits and systems disclosed herein provide for monitoring a disease or condition in a subject, or administering a test for a disease or condition. Some methods disclosed herein comprise treating a subject for a disease or condition, monitoring a disease or condition in a subject, or administering a test for a disease or condition. In some instances, the methods disclosed herein comprise determining the subject has a disease or condition, thereby informing the subject or their healthcare provider that a treatment or test would be appropriate, suitable or beneficial to the subject. In some instances, the methods disclosed herein comprise determining the subject has a disease or condition and recommending a treatment for the disease or condition. In some instances, the methods disclosed herein comprise determining the subject has a disease or condition and treating the subject for the disease or condition. In some instances, the methods disclosed herein comprise determining the subject has a disease or condition and monitoring the subject for the disease or condition. In some instances, the methods disclosed herein comprise determining the subject has an increased risk or possibility of having the disease or condition relative to an individual without the disease or condition, and administering a test specific for the disease or condition to the subject. In some instances, the methods disclosed herein comprise determining the subject has an increased risk or possibility of having the disease or condition relative to an individual without the disease or condition, and recommending a test specific for the disease or condition to the subject.

Provided herein are therapeutic agents, compositions, compounds and agents for the treatments of diseases and conditions. One of skill in the art would understand that combination and analogs of these agents are contemplated and intended herein even if each combination and analog is not explicitly described. An “analog,” as used herein refers to a modified or synthetic compound that resembles a naturally-occurring compound, wherein at least 50% of the analog structure is identical to at least 50% of the naturally-occurring compound.

Methods disclosed herein may comprise treating a subject for a liver-associated disease with a therapy or therapeutic agent. The liver-associated disease, by way of non-limiting example, may be selected from NAFLD, NASH, hepatitis, and cirrhosis. The therapy or therapeutic agent may be selected from compounds, both naturally occurring and synthetic, that are disclosed herein, as well as analogs thereof.

Methods disclosed herein may comprise treating a subject for NAFLD or NASH. Treating the subject for NAFLD or NASH may comprise administering or recommending a therapy to the subject selected from weight loss, dietary limitations (e.g., reduced sugar/fat/cholesterol intake), and bariatric surgery. Treating the subject for NAFLD may comprise administering or recommending an appetite suppressant. A non-limiting example of an appetite suppressant use to treat NAFLD or NASH is sibutramine or an analog thereof. Treating the subject for NAFLD may comprise administering a pharmaceutical targeting steatosis or insulin resistance. By way of non-limiting example, a pharmaceutical targeting steatosis or insulin resistance is metformin or an analog thereof. A pharmaceutical targeting steatosis or insulin resistance may be a statin. The statin, by way of non-limiting example, may be selected from atorvastatin, pravastatin, rosuvastatin, and tetrahydrolipstatin, or an analog thereof. A pharmaceutical targeting steatosis or insulin resistance may be a fibrate, e.g., gemfibrozil or an analog thereof. A pharmaceutical targeting steatosis or insulin resistance may be a thiazolidinedione or peroxisome proliferator activated receptor (PPAR) agonist (e.g., pioglitazone, rosiglitazone, GFT505 or an analog thereof). A pharmaceutical targeting steatosis or insulin resistance may be a bile acid or analog thereof, e.g., ursodiol, 6α-ethyl-chenodeoxycholic acid. A pharmaceutical targeting steatosis or insulin resistance may be a vitamin or analog thereof, e.g., vitamin E, vitamin D or vitamin C, or analogues thereof. Treating the subject for NAFLD or NASH may comprise administering or recommending a caspase inhibitor (e.g., IDN-6556, PF-03491390 or an analog thereof). Treating the subject for NAFLD or NASH may comprise administering or recommending an antioxidant, anti-inflammatory or anti-cytokine (e.g., pentoxifylline, betaine or an analog thereof). Treating the subject for NAFLD or NASH may comprise administering or recommending a pro-biotic or pre-biotic. Treating the subject for NAFLD or NASH may comprise administering or recommending a fibrosis inhibitor. By way of non-limiting example, the fibrosis inhibitor may be an angiotensin II receptor antagonist (e.g., losartan or an analog thereof).

Methods disclosed herein may comprise treating a subject for NASH or selecting a treatment for NASH. NASH is generally described in the field as a severe form of NAFLD, a significantly developed form of NAFLD or an aggressive condition of NAFLD. Scarring of the liver, progressing to cirrhosis (long term damage), may occur in subjects with NASH, but generally does not occur in subjects with NAFLD. NASH may have significantly more inflammation and deposition of extracellular matrix components as compared to NAFLD. At least partially for these reasons, the methods, kits and systems disclosed herein may comprise use of the same markers and liver-specific polynucleotides for detecting NAFLD and NASH in the subject. However, the levels of these markers and tissue-specific polynucleotides may be different in each of these conditions. Liver-specific polynucleotides may be different in each of these conditions due to the fact that more cell death resulting in release of more liver-specific polynucleotides may occur in the more severe case of NASH. In some instances, the methods comprise quantifying a marker of NAFLD or NASH and/or a liver-specific polynucleotide, and determining a subject has NASH if a level of the marker and/or liver-specific polynucleotide is at least 20% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 30% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 40% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 50% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 60% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 70% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 80% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 90% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 100% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 150% greater than an average level of the marker and/or liver-specific polynucleotide in NAFLD subjects. In some instances, the methods comprise quantifying a marker and/or liver-specific polynucleotide of NAFLD or NASH, and determining a subject has NASH if a level of the marker and/or liver-specific polynucleotide is at least 200% greater than an average level of the marker and/or liver-specific polynucleotide in NAFLD subjects.

Methods disclosed herein may further comprise administering a test for NAFLD or NASH. The test may be specific for NAFLD or NASH. The test specific for NAFLD or NASH may be a liver function test, which measure levels of enzymes made by liver cells (e.g., aspartate aminotransferase (AST or SGOT) and alanine aminotransferase (ALT or SGPT)). Tests for NAFLD or NASH may include a right upper quadrant ultrasound examination, skin color examination (yellowing of skin), or a liver scan (e.g., ultrasound, CT or MRI). The methods may comprise monitoring the subject for progression of or progression to NAFLD or NASH using a method or test disclosed herein. The subject may be overweight. The subject may be obese. The subject may have a body mass index (BMI) greater than 25. The subject may have a BMI greater than 30. The subject may be insulin resistant. The subject may be insulin insensitive. The subject may have diabetes. The subject may have type II diabetes. Monitoring the subject for progression of or progression to NAFLD or NASH may comprise detecting or quantifying a marker for NAFLD or NASH and/or a liver-specific polynucleotide more than one time. Monitoring the subject for progression of or progression to NAFLD or NASH may comprise detecting or quantifying a marker NAFLD and/or a liver-specific polynucleotide after treating the subject for NAFLD or NASH.

Provided herein are methods that comprise treating a subject for a hepatitis or symptoms thereof or selecting a treatment for a hepatitis (symptom). The hepatitis may be acute hepatitis. The hepatitis may be chronic hepatitis. The hepatitis may be a viral hepatitis or an alcohol-induced hepatitis. The viral hepatitis may be selected from Hepatitis A, Hepatitis B, or Hepatitis C. Treating the subject for a viral hepatitis may comprise reducing medications or alcohol intake, allowing the liver to heal. Treating the subject for a viral hepatitis may comprise administering an interferon to the subject. Treating the subject for a viral hepatitis may comprise administering an antiviral agent to the subject. Treating the subject for hepatitis C may comprise administering ribavirin to the subject. Treating the subject for Hepatitis C may comprise administering a protease inhibitor to the subject. By way of non-limiting example, the protease inhibitor may be selected from boceprevir, simeprevir, sofosbuvir, dacatasvir, ledipasvir, ombitasvir, paritaprevir, and ritonavir, and analogs thereof. Treating the subject for Hepatitis B may comprise administering a therapy to the subject selected from injectable alpha interferons, lamivudine, adefovir, entecavir, telbivudein, and tenofovir, and analogs thereof, and combinations thereof. The methods may comprise administering a test for a hepatitis disclosed herein. The test may be specific for a hepatitis disclosed herein. Tests for hepatitis may include an antibody-based blood test for antigens of Hepatitis strains. The methods may comprise monitoring the subject for progression of hepatitis. Monitoring the subject for progression of hepatitis may comprise detecting or quantifying a marker for hepatitis and/or a liver-specific polynucleotide more than one time. Monitoring the subject for progression of hepatitis may comprise detecting or quantifying a marker for hepatitis and/or a liver-specific polynucleotide after treating the subject for hepatitis.

Methods disclosed herein may comprise treating the subject for cirrhosis or selecting a treatment for cirrhosis. Treatments for cirrhosis may comprise administering a therapy to the subject selected from, but not limited to a transjugular intrahepatic portosystemic shunt (TIPS) to lower fluid buildup in liver, liver transplantation, ursodiol, and obeticholic acid. The methods may comprise administering a test for cirrhosis. The methods may comprise a test used in monitoring the subject for progression of cirrhosis or response to a treatment disclosed herein. The test may be specific for cirrhosis. Tests for cirrhosis may include, but are not limited to MRI, CT scan, ultrasound, biopsy, blood test for excess bilirubin or creatinine, and the international normalized ratio (INR) test for clotting. Monitoring the subject for progression of cirrhosis may comprise detecting or quantifying a marker for cirrhosis and/or a liver-specific polynucleotide more than one time. Monitoring the subject for progression of cirrhosis may comprise detecting or quantifying a marker for cirrhosis and/or a liver-specific polynucleotide after treating the subject for cirrhosis.

Methods disclosed herein may comprise treating a subject for a cardiovascular disease. The cardiovascular disease, by way of non-limiting example, may be selected from atherosclerosis, atheroma, coronary artery disease and diabetic ischemic cardiomyopathy. Treating the subject for the cardiovascular disease may comprise administering a therapy to the subject selected from a statin, a blood thinner, an antioplasty/stent, a beta blocker, a calcium channel blocker, a fibrinolytic therapy, a tissue plasminogen activator, nitroglycerin, an acetylcholine esterase (ACE) inhibitor, or a combination thereof. Treating the subject for the cardiovascular disease may comprise performing a bypass surgery, bypass graft surgery or percutaneous coronary revascularization on the subject. Statins, by way of non-limiting example, include lovastatin, atorvastatin, fluvastatin, pitavastatin, pravastatin, rosuvastatin and simvastatin. Fibrinolytic therapies, by way of non-limiting example, include streptokinase, urokinase, and anistreplase. Tissue plasminogen activators, include, but are not limited to, alteplase, reteplase, tenecteplase and staphylokinase.

Methods disclosed herein may comprise a test used in monitoring the subject for progression of a cardiovascular disease or response to a treatment disclosed herein. The test may be specific for the cardiovascular disease. Tests for a cardiovascular disease may include, but are not limited to an angiography, a carotid intima-media thickness scan (CIMT), intravascular ultrasound (IVUS), a CT scan, an echocardiogram or echocardiography (ECG/EKG), chest x-ray, stress test, coronary angiography and cardiac catheterization. Monitoring the subject for progression of cirrhosis may comprise detecting or quantifying a marker for a cardiovascular disease more than one time. Monitoring the subject for progression of a cardiovascular disease may comprise detecting or quantifying a marker for a cardiovascular disease and/or a heart-specific, artery-specific, or endothelium-specific polynucleotide after treating the subject for the cardiovascular disease. Monitoring the subject for progression of the cardiovascular disease may comprise detecting or quantifying a marker for the cardiovascular disease and/or a cardiovascular-specific polynucleotide more than one time. Monitoring the subject for progression of atherosclerosis may comprise detecting or quantifying a marker for the cardiovascular disease and/or a cardiovascular-specific polynucleotide after treating the subject for the cardiovascular disease. The cardiovascular-specific polynucleotide may be a polynucleotide predominantly or specifically expressed in an aorta. The cardiovascular-specific polynucleotide may be a polynucleotide predominantly or specifically expressed a coronary artery. The cardiovascular-specific polynucleotide may be a polynucleotide predominantly or specifically expressed in endothelial cells. The cardiovascular-specific polynucleotide may be a polynucleotide predominantly or specifically expressed in vascular smooth muscle cells.

Provided herein are methods, systems and kits for the treatment of diabetic ischemic cardiomyopathy. Treating the subject for diabetic ischemic cardiomyopathy may comprise administering a therapy to the subject selected from insulin, metformin, pioglitazone, dapagliflozin, GLP-1 mimetics/agonists, dipeptidyl peptidease-4 (DPP-4) inhibitors, amylin analogues, statins, vasoactive agents, phosphodiesterase type 5 inhibitors, antioxidants (e.g., trimetazidine). The methods may comprise administering a test for diabetic ischemic cardiomyopathy. The test may be specific for diabetic ischemic cardiomyopathy. Tests for diabetic ischemic cardiomyopathy include, but are not limited to, an echocardiography, an MRI, a coronary angiography, SPECT imaging, PET imaging, or a cardiac catheterization. The methods may comprise monitoring the subject for progression of diabetic ischemic cardiomyopathy. Monitoring the subject for progression of diabetic ischemic cardiomyopathy may comprise use of tests disclosed herein. Monitoring the subject for progression of diabetic ischemic cardiomyopathy may comprise detecting or quantifying a marker for diabetic ischemic cardiomyopathy and/or a cardiovascular-specific polynucleotide more than one time. Monitoring the subject for progression of diabetic ischemic cardiomyopathy may comprise detecting or quantifying a marker for diabetic ischemic cardiomyopathy and/or a cardiovascular-specific polynucleotide after treating the subject for diabetic ischemic cardiomyopathy. Markers for diabetic ischemic cardiomyopathy include, but are not limited to matrix metalloproteases, cardiac tropins and an N-terminal procollagen III propeptide (PIIINP).

Methods disclosed herein may comprise treating a subject for a cancer. Treating the subject for the cancer may comprise administering a therapy to the subject selected from a radiation therapy, a chemotherapy, a cell-based therapy, an immunotherapy, and a combination thereof. In some instances, the cancer is treated with a kinase inhibitor. The kinase inhibitor may be a tyrosine kinase inhibitor. The kinase inhibitor may be a serine kinase inhibitor. The kinase inhibitor may be a threonine kinase inhibitor.

Treating the subject for the cancer may comprise administering a cell-based therapy to the subject. The term “cell-based therapy,” as used herein is a therapy that uses a targeting cell, such as a T cell, to attack a target cell, such as a cancer cell. The targeting cell may be autologous or allogeneic. The targeting cell may be engineered, partially engineered or genetically modified. The targeting cell may be a lymphocyte, e.g., a macrophage, a T cell or a natural killer cell. In some cases an antibody, antigen-binding antibody fragment, small molecule, peptide, or combination thereof that binds the target cell and targeting cell, bringing them in proximity for the targeting cell to have cytotoxic effects on the target cell.

The term “immunotherapy,” as used herein is a therapy that utilizes the immune system or component thereof. Generally immunotherapies disclosed herein comprise an immunoglobulin (antibody) or antigen-binding fragment thereof. In some cases, the antibody is conjugated to a drug that is cytotoxic to a target cell, and the antibody binds an antigen on the target cell, thereby bringing the drug in proximity of the target cell. In some cases the antibody is conjugated to a peptide, ligand or small non-drug molecule that binds a cell surface molecule of the target cell, and the antibody binds to the targeting cell, thereby bringing a targeting cell in proximity of the target cell. In some cases, the antibody is not conjugated to anything and blocks activity or interaction of a cell surface molecule on the target cell. The antibody may thereby reduce the target cell's growth, metastasis or interaction with other cells.

Provided herein are methods, kits and systems for treating a subject with a breast cancer. Further provided herein are methods, kits and systems for selecting a therapy or process for treating a subject with a breast cancer. Treating the subject for breast cancer may comprise performing a lumpectomy, mastectomy, irradiation therapy on the subject. Treating the subject for breast cancer may comprise administering a therapy to the subject selected from capecitabine, carboplatin, cisplatin, cyclophosphamide, docetaxel, (pegylated liposomal) doxorubicin, epirubicin, fluorouracil, gemcitabine, methotrexate, (protein bound) paclitaxel, vinorelbine, eribulin, ixabepilone, and combinations thereof. The breast cancer may be Her2 positive and the treatment may be lapatinib or palbociclib. The therapy may be a hormonal therapy. By way of non-limiting example, the hormonal therapy may be tamoxifen, an aromatase inhibitor or fulvestrant. The therapy may be an immunotherapy. Non-limiting examples of immunotherapies for breast cancer include trastuzumab, pertuzumab, and conjugates thereof. Monitoring the subject for breast cancer or a response to treatment of breast cancer may comprise performing a lymph node biopsy, a lymph node dissection, a breast exam, a mammogram, an ultrasound, an MRI, a breast biopsy, or a combination thereof, on the subject.

Provided herein are methods, kits and systems for treating a subject with a prostate cancer. Further provided herein are methods, kits and systems for selecting a therapy or process for treating a subject with a prostate cancer. Treating the subject for prostate cancer may comprise performing prostatectomy, radiation treatment (external beam, brachytherapy, orchiectomy), cryosurgery, cryoablation, high intensity focused ultrasound on the subject. Treating the subject for prostate cancer may comprise administering a therapy to the subject selected from leuprolide, goserelin, triptorelin, histrelin, ketoconazole, abiraterone, bicalutamide, flutamide, nilutamide, enzalutamide or a combination thereof, to the subject. The therapy may be an immunotherapy. The immunotherapy may bind an antigen encoded by a gene selected from PSA or CTLA-4. The immunotherapy may comprise primary dendritic cells incubated with a PAP/GM-CSF fusion protein. Non-limiting examples of immunotherapies for prostate cancer include sipuleucel-T, Prostvac, GVAX, and ipilimumab. Monitoring the subject for prostate cancer or a response to treatment of prostate cancer may comprise performing active surveillance and imaging scans of the subject.

Provided herein are methods, kits and systems for treating a subject with a lung cancer. Further provided herein are methods, kits and systems for selecting a therapy or process for treating a subject with a lung cancer. Treating the subject for lung cancer may comprise performing a lobectomy, wedge resection, segmentectomy, pneumonectomy, radiation therapy, or chemotherapy on the subject. Treating the subject for lung cancer may comprise administering a therapy to the subject selected from carboplatin, cisplatin, docetaxel, gemcitabine, nab-paclitaxel, pemetrexed, vinorelbine, crizotinib, ceritinib, alectinib, or a combination thereof, to the subject. Treating the subject for lung cancer may comprise administering an epidermal growth factor receptor (EGFR) inhibitor to the subject. Non-limiting examples of EGFR inhibitors are erlotinib, gefitinib, afatinib, and osimertinib. The therapy may be an immunotherapy. The immunotherapy may inhibit angiogenesis. Non-limiting examples of immunotherapies that inhibit angiogenesis include bevacizumab and ramucirumab. The immunotherapy may bind an antigen encoded by a gene selected from PD-1, DLL3 and EGFR. Non-limiting examples of immunotherapies for lung cancer include Nivolumab, pembrolizumab, rovalpituzumab tesirine, and necitumumab. Monitoring the subject for lung cancer or a response to treatment of lung cancer may comprise performing a CT scan, a biopsy, an MRI, a bronchoscopy, a mediastinoscopy, a mediastinotomy, an endobronchial ultrasound, an endoscopic esophageal ultrasound, a thoracentesis, a thoracoscopy or video-assisted thoracic surgery, a sputum cytology, or a fine needle aspiration (FNA).

Provided herein are methods, kits and systems for treating a subject with a colon cancer. Further provided herein are methods, kits and systems for selecting a therapy or process for treating a subject with a colon cancer. Treating the subject for colon cancer may comprise performing a polyp removal, endoscopic mucosal resection, partial colectomy, radiation, targeted chemotherapy, or colostomy on the subject. Treating the subject for colon cancer may comprise administering an inhibitor of VEGFR or TIE2 activity (e.g., regorafenib) to the subject. Treating the subject for colon cancer may comprise administering an immunotherapy to the subject. The immunotherapy may bind an antigen encoded by a gene selected from a VEGF, a VEGFR, or an EGFR. The VEGF may be VEGF-A. The VEGFR may be VEGR2. Non-limiting examples of immunotherapies for colon cancer include bevacizumab, cetuximab, panitumumab, ramucirumab, and ziv-aflibercept. Monitoring the subject for colon cancer or a response to treatment of colon cancer may comprise performing a lymph node removal or biopsy, a colonoscopy, a biopsy, a CT scan, an MRI, an ultrasound, an x-ray or a PET scan on the subject

Provided herein are methods, kits and systems for treating a subject with a uterine cancer. Further provided herein are methods, kits and systems for selecting a therapy or process for treating a subject with a uterine cancer. Treating the subject for uterine cancer may comprise performing radiation and/or a hysterectomy on the subject. Treating the subject for uterine cancer may comprise administering a therapy for breast cancer disclosed herein. Treating the subject for uterine cancer may comprise administering an aromatase inhibitor to the subject. Non-limiting examples of aromatase inhibitors include anastrozole, letrozole and exemestane. Treating the subject for uterine cancer may comprise administering an immunotherapy to the subject. The immunotherapy may bind an antigen encoded by a gene selected from PD-1 and CTLA-4. Non-limiting examples of immunotherapies for uterine cancer include Nivolumab and ipilimumab. Monitoring the subject for uterine cancer or a response to treatment of uterine cancer may comprise performing a PAP test, a lymph node biopsy/dissection, a transvaginal ultrasound, a CT scan, an MRI, or an endometrial biopsy on the subject.

Provided herein are methods, kits and systems for treating a subject with a bladder cancer. Further provided herein are methods, kits and systems for selecting a therapy or process for treating a subject with a bladder cancer. Treating the subject for bladder cancer may comprise performing surgery or radiation on the subject. Treating the subject for bladder cancer may comprise administering a chemotherapy to the subject. Treating the subject for bladder cancer may comprise administering an immunotherapy to the subject. The immunotherapy may bind an antigen encoded by a gene selected from IFNAR1 or IFNAR2c, (e.g., synthetic interferon alfa-2b). Treating the subject for bladder cancer may comprise administering a biological therapy to the subject. The biological therapy may be Bacille Calmette-Guerin (BCG), a bacterium normally used as a TB vaccine, but also a highly successful immunotherapy for bladder cancer. Monitoring the subject for bladder cancer or a response to treatment of bladder cancer may comprise performing a biopsy, an x-ray, a CT scan, a bone scan, ultrasound, MRI or PET scan. Monitoring the subject for bladder cancer or a response to treatment of bladder cancer may comprise performing a cystoscopy, a transurethral resection of a bladder tumor, an intravenous pyelogram, or a retrograde pyelogram. Monitoring the subject for bladder cancer or a response to treatment of bladder cancer may comprise quantifying markers of bladder cancer in a urinalysis. Non-limiting examples of markers of bladder cancer include bladder tumor-associated antigen (BTA/CFHrp), mucin, carcinoembryonic antigen (CEA), and NMP22 protein.

Provided herein are methods, kits and systems for treating a subject with a skin cancer. Further provided herein are methods, kits and systems for selecting a therapy or process for treating a subject with a skin cancer. Treating the subject for skin cancer may comprise performing a surgery or radiation on the subject. Treating the subject for skin cancer may comprise administering a chemotherapy to the subject. Non-limiting examples of chemotherapy include dacarbazine, temozolomide, nab-paclitaxel, paclitaxel, cisplatin, carboplatin, vinblastine, and combinations thereof. Treating the subject for skin cancer may comprise administering a chemotherapy to the subject that inhibits B-Raf or MEK activity. Treating the subject for skin cancer may comprise administering a chemotherapy to the subject selected from vemurafenib, dabrafenib, trametinib, or a combination thereof, to the subject. Treating the subject for skin cancer may comprise administering an immunotherapy to the subject. The immunotherapy may bind an antigen encoded by a gene selected from IL-2R, CTLA-4, and PD-1. Non-limiting examples of immunotherapies for skin cancer include interferon alpha, interleukin-2, ipilimumab, nivolumab, and pembrolizumab. Monitoring the subject for skin cancer or a response to treatment of skin cancer may comprise performing a skin biopsy, MRI, PET scan, lymph node biopsy, or chest x-ray on the subject. Monitoring the subject for skin cancer or a response to treatment of skin cancer may comprise obtaining reflectance confocal micrographs of the subject. Monitoring the subject for skin cancer or a response to treatment of skin cancer may comprise quantifying levels of lactate dehydrogenase (LDH) in a blood sample of the subject.

Provided herein are methods, kits and systems for treating a subject with a thyroid cancer. Further provided herein are methods, kits and systems for selecting a therapy or process for treating a subject with a thyroid cancer. Treating the subject for thyroid cancer may comprise performing surgery or radiation therapy on the subject. Treating the subject for thyroid cancer may comprise administering a chemotherapy to the subject. Treating the subject for thyroid cancer may comprise administering a therapy to the subject selected from cabozantinib-s-malate, carelsa, cometriq, doxorubicin hydrochloride, lenvatinib mesylate, lenvima, nexavar, sorafebnib tosylate and or a combination thereof, to the subject. Treating the subject for thyroid cancer may comprise administering a therapy to the subject that inhibits an EGFR or a RET tyrosine kinase. Treating the subject for thyroid cancer may comprise administering a therapy to the subject selected from vandetanib and sorafebnib. Treating the subject for thyroid cancer may comprise administering an immunotherapy to the subject. The immunotherapy may bind an antigen encoded by a PD-1 gene. The immunotherapy may be pembrolizumab. Monitoring the subject for thyroid cancer or a response to treatment of thyroid cancer may comprise performing an ultrasound, biopsy, physical examination, x-ray, CT scan or PET scan on the subject. Monitoring the subject for skin cancer or a response to treatment of skin cancer may comprise quantifying levels of T3, T4, TSH, Tg, TgAb, or CEA in a blood sample of the subject. Monitoring the subject for thyroid cancer or a response to treatment of thyroid cancer may comprise performing radionuclide scanning with radioisotope I-131 or I-123.

Provided herein are methods, kits and systems for treating a subject with a lymphoma. Further provided herein are methods, kits and systems for selecting a therapy or process for treating a subject with a lymphoma. Treating the subject for a lymphoma, such as Non Hodgkin Lymphoma, may comprise performing a radiation or a stem cell transplant on the subject. Treating the subject for lymphoma may comprise administering a chemotherapy to the subject. By way of non-limiting example, the chemotherapy may be selected from cyclophosphamide, doxorubicin, vincristine and prednisone. Treating the subject for lymphoma may comprise administering an immunotherapy to the subject. The immunotherapy may bind an antigen encoded by a gene selected from CD20. Non-limiting examples of immunotherapies for lymphoma include rituximab and ibritumomab tiuxetan. Monitoring the subject for lymphoma or a response to treatment of lymphoma may comprise performing a physical exam, CT scan, PET scan, MRI or biopsy on the subject. The biopsy may be a lymph node biopsy or a bone marrow biopsy.

Provided herein are methods, kits and systems for treating a subject with a leukemia. Further provided herein are methods, kits and systems for selecting a therapy or process for treating a subject with a leukemia. Treating the subject for a leukemia, such as acute lymphocytic leukemia (ALL), acute myelogenous leukemia (AML) or chronic lymphocytic leukemia (CLL) may comprise performing radiation or a stem cell transplant on the subject. Treating the subject for leukemia may comprise administering a chemotherapy to the subject. Non-limiting examples of chemotherapies for leukemia include vincristine, daunorubicin, doxorubicin, cytarabine, L-asapraginase, PEG-L-asparaginase, etoposide, teniposide, 6-mercaptopurine, methyotrexate, cyclophosphamide, prednisone, dexamethasone, prednisone, cladribine, fludarabine, topotecan, etoposide, 6-thioguanine, hydroxyurea, methotrexate, azacitidine, decitabine, bendamustine, pentostatin, and ibrutinib, and combinations thereof. In some cases, the leukemia is ALL and the chemotherapy is imatinib, dasatinib, nilotinib or blinatumomab. In some cases, the leukemia is AML and the chemotherapy is arsenic trioxide or all-trans retinoic acid. In some cases, the leukemia is CLL and the chemotherapy is ibrutinib, idelalisib or lenalidomide. Treating the subject for leukemia may comprise administering an immunotherapy to the subject. By way of non-limiting example, the immunotherapy may target an antigen selected from, CD3, CD19, CD20, CD33, CD52, PD-L1, and CTLA-4. The leukemia may be ALL and the immunotherapy may be selected from blinatumomab, rituximab, ofatumumab, obinutuzumab, and alemtuzumab. The leukemia may be AML and the immunotherapy may be selected from gemtuzumab ozogamicin, a chimeric antigen receptor expressing T cell that binds CD19 on the leukemia cell, and ipilimumab. The leukemia may be CLL and the immunotherapy may be selected from alemtuzumab, ofatumumab, obinutuzumab, and rituximab. Monitoring the subject for leukemia or a response to treatment of leukemia may comprise performing blood cell count, a bone marrow aspiration or biopsy, an x-ray, CT-scan, ultrasound, a peripheral blood smear, cytogenetic analysis, immunophenotyping (cell flow cytometry to characterize relative quantities of cell types in blood), a lumbar puncture, or a spinal fluid test, or a combination thereof.

Provided herein are methods, kits and systems for treating a subject with a renal disease. Further provided herein are methods, kits and systems for selecting a therapy or process for treating a subject with a renal disease. Treating the subject for a renal disease may comprise performing dialysis or a kidney transplant on the subject. Treating the subject for renal disease may comprise administering a therapy to the subject selected from an angiotensin converting enzyme (ACE) inhibitor, an angiotensin II receptor blocker, a blood pressure lowering medication, a diuretic, and a combination thereof, to the subject. The therapy may be an immunotherapy. Monitoring the subject for renal disease or a response to treatment of renal disease may comprise performing imaging or biopsy of the subject. Monitoring the subject for renal disease or a response to treatment of renal disease may comprise quantifying markers of renal disease more than once. Markers for the renal disease include, but are not limited to, creatine and urea. Markers may be detected or quantified in whole blood or urine.

Provided herein are methods, kits and systems for treating a subject with a retinal disorder or disease. Further provided herein are methods, kits and systems for selecting a therapy or process for treating a subject with a retinal disorder or disease. Treating the subject for a retinal disorder or disease may comprise performing a laser treatment, cryopexy, retinopexy, proton beam therapy, scleral buckle, vitrectomy or intraocular injection of an eye of the subject. Treating the subject for a retinal disorder or disease may comprise administering a therapy to the subject selected from a biologic, e.g., interfering RNA or a small molecule drug (e.g., pegaptanib) to the subject. The therapy may be an immunotherapy. The immunotherapy may regulate vascularization of the eye. The immunotherapy may target (e.g., bind) VEGF or VEGF receptors. In some cases, the immunotherapy targets a VEGF-A or a VEGF-A receptor. Non-limiting examples of immunotherapies for a retinal disorder include ranibizumab and bevacizumab.

### Kits & Systems

As discussed in the foregoing and following description, systems and kits are provided herein to non-invasively detect a tissue or organ in a subject that is under duress as well as determine which disease or condition is affecting the tissue or organ under duress. Disclosed herein are kits for use in detecting a disease or condition in a subject, the kit comprising at least one reagent for detecting at least one marker, and at least one reagent for detecting at least one tissue-specific polynucleotide. Additionally or alternatively, the kits disclosed herein may be used to determine the location (e.g., tissue) and/or progression of a disease or condition in the subject. Additionally or alternatively, the kits disclosed herein may be used to determine if a therapy administered to the subject has affected the progression or stage of the disease or condition. Additionally or alternatively, the kits disclosed herein may be used to determine if a therapy administered to the subject has resulted in any unintended toxicity or side effects.

Provided herein are kits that comprise at least one reagent disclosed herein. The at least one reagent for detecting tissue-specific polynucleotides may comprise at least one reagent for detecting a cell-free polynucleotide. The at least one reagent for detecting at least one marker may comprise at least one reagent for a detecting cell-free polynucleotide. The at least one cell free polynucleotide may comprise cell-free DNA or cell-free RNA. The cell-free DNA may have a tissue-specific methylation pattern. The cell free polynucleotide may be a tissue-specific gene transcript. The at least one reagent for detecting at least one marker and/or the at least one reagent for detecting the tissue-specific polynucleotide may comprise a polynucleotide probe. The polynucleotide probe may bind to the cell-free polynucleotide. The polynucleotide probe may bind to the cell-free polynucleotide in a sequence-dependent manner. The polynucleotide probe may bind to a cell-free polynucleotide corresponding to a wildtype version of a gene, but not a mutant version of the gene. Alternatively, the polynucleotide probe may bind to a cell-free polynucleotide corresponding to a mutant version of a gene, but not a wildtype version of the gene. The polynucleotide probe may be attached to a signaling moiety. By way of non-limiting example, the signaling moiety may be selected from a hapten, a fluorescent molecule, and a radioactive isotope. The kit may be specific for one disease or condition. The kit may comprise as few as 1, 2, 3, 4, or 5 polynucleotide probes in order to detect a disease or condition in a subject. The kit may be specific for multiple diseases or conditions. The kit may comprise from about 5 to about 10, about 10 to about 20, about 10 to about 100, about 10 to about 1000, about 100 to about 1000, or about 100 to about 10,000 polynucleotide probes.

Provided herein are kits that comprise at least one reagent disclosed herein. The at least one reagent for detecting at least one marker and/or the at least one reagent for detecting the tissue-specific polynucleotide may comprise a primer. The primer may be a reverse transcriptase primer. The primer may be a PCR primer. The primer may amplify the at least one marker, at least one tissue-specific polynucleotide, or portions thereof. The primer may amplify the cell-free polynucleotide in a sequence-dependent manner. The primer may amplify a cell-free polynucleotide or portion thereof corresponding to a wildtype version of a gene, but not a mutant version of the gene. Alternatively, the primer may amplify a cell-free polynucleotide or portion thereof corresponding to a mutant version of a gene, but not a wildtype version of the gene. The kit may further comprise an amplification reporter that provides a user of the kit with the quantity of the at least one marker and/or the at least one reagent for detecting the tissue-specific polynucleotides. Typically, the quantity is a relative quantity based on a reference sample. The amplification signaling reagent may be selected from intercalating fluorochromes or dyes. The amplification signaling reagent may be SYBR Green.

Provided herein are kits that comprise at least one reagent disclosed herein. The at least one reagent for detecting at least one marker and/or the at least one reagent for detecting the tissue-specific polynucleotide may comprise a peptide that binds to the at least one marker or tissue-specific polynucleotide. The peptide may be part of an antibody, or a polynucleotide binding protein (e.g., transcription factor, histone). The at least one reagent for detecting at least one marker and/or the at least one reagent for detecting the tissue-specific polynucleotide may comprise a signaling moiety that emits a signal, wherein the signal being emitted or lost is indicative of a presence or a quantity of a marker or a tissue-specific polynucleotide. Examples of signaling moieties include, but are not limited to, dyes, fluorophores, enzymes and radioactive particles. The at least one reagent may further comprise a signaling moiety detector for detecting the signal or absence thereof.

Disclosed herein are kits for use in detecting a disease or condition in a subject, the kit comprising at least one reagent for detecting at least one marker, and at least one reagent for detecting at least one tissue-specific polynucleotide. The kit may further comprise a solid support, wherein the polynucleotide probe, the primer and/or the peptide is attached to a solid support. The solid support may be selected from a bead, a chip, a gel, a particle, a well, a column, a tube, a probe, a slide, a membrane, and a matrix.

Disclosed herein are kits for use in detecting a disease or condition in a subject, the kit comprising at least one reagent for detecting at least one marker, and at least one reagent for detecting at least one tissue-specific polynucleotide. Two or more components of the kits disclosed herein may be separate. Two or more components of the kits disclosed herein may be integrated. Two or more components of the kits disclosed herein may be integrated into a device. The device may allow for a user to simply add at least one sample from the subject to the device and receive a result indicating whether or not the subject has the disease or condition and/or which tissue(s) of the subject is affected by the disease or condition. In some cases, the user may add at least one reagent to the device. In other cases, the user does not have to add any reagents to the device.

Disclosed herein are kits for use in detecting a disease or condition in a subject, the kit comprising at least one reagent for detecting at least one marker, and at least one reagent for detecting at least one tissue-specific polynucleotide. The at least one tissue-specific polynucleotide or marker may comprise a cell free polynucleotide. The at least one marker may comprise RNA. The at least one tissue-specific polynucleotide may comprise at least one tissue specific RNA, wherein a tissue specific RNA is an RNA expressed only in a specific tissue or at a level in a specific tissue that is substantially higher than the level at which it is expressed in other tissues. For example, a tissue specific gene may be a gene for which expression in a particular tissue or group of tissues is at least 2-fold, 5-fold, 10-fold, or 25-fold greater than any other tissue or group of tissues (e.g. any individually, or all other tissues or group of tissues combined). The at least one tissue-specific polynucleotide or marker may comprise at least one tissue-specific methylated DNA, wherein the tissue-specific methylated DNA comprises a tissue-specific methylation pattern. Alternatively or additionally, the tissue-specific methylated DNA may comprise DNA with a methylation pattern that occurs in only one tissue or at a level in a tissue that is substantially higher than the level at which it occurs in other tissues. The tissue may be determined to be damaged by the condition if (a) the level of at least one of the marker is above the reference level of the at least one marker, and (b) the level of at least one of the tissue-specific polynucleotide is above the reference level of the at least one tissue-specific polynucleotide. The at least one tissue-specific polynucleotide may comprise two or more polynucleotides each of which is specific for a different tissue (e.g. 2, 3, 4, 5, 10, 15, 25, or more different tissues). The tissue may be at least one of: whole blood, bone, epithelium, hypothalamus, smooth muscle, lung, thymus, lymph node, thyroid, heart, kidney, brain, cerebellum, liver, and skin. The marker and/or tissue-specific polynucleotide may correspond to a gene. In general, a marker or tissue-specific polynucleotide “corresponds to a gene” if it is a DNA molecule comprising the gene (or an identifiable portion thereof), or is an expression product of the gene (e.g. an RNA transcript or a protein product).

Disclosed herein are kits for use in detecting a kidney under duress, wherein the kits comprise at least one primer or probe for a kidney-specific polynucleotide. Further disclosed herein are kits for detecting the presence of a disease in a kidney, wherein the kits comprise at least one primer or probe for a kidney-specific polynucleotide. Further disclosed herein are kits for detecting damage to a kidney, wherein the kits comprise at least one primer or probe for a kidney-specific polynucleotide. Further disclosed herein are kits for detecting damage to a kidney, wherein the kits comprise at least three primers or probes for a kidney-specific polynucleotide. Further disclosed herein are kits for detecting damage to a kidney, wherein the kits comprise at least five primers or probes for a kidney-specific polynucleotide. The kidney-specific polynucleotide may correspond to a gene selected from the group consisting of: AK3L1, AQP2, BBOX1, BFSP2, BHMT, C20ORF194, CA12, CDH16, CLCNKA, CRYAA CTXN3, CUBN, DDC, EGF, ENPEP, FMO1, FOLR3, FUT3, FXYD2, GGT1, HAO2, HKID, HNF1B, KCNJ1, KL, NAT8, NOX4, PDZK1, PDZK1IP1, PTH1R, RBP5, SLC12A1, SLC12A3, SLC13A3, SLC17A3, SLC22A2, SLC22A6, SLC22A8, SLC34A1, SLC3A1, SLC6A13, SLC7A7, SLC7A8, SLC7A9, TREH, UGT1A1, UGT1A6, UMOD, and XPNPEP2, and combinations thereof.

Disclosed herein are kits for use in detecting a liver under duress, wherein the kits comprise at least one primer or probe for a liver-specific polynucleotide. Further disclosed herein are kits for detecting the presence of a disease in a liver, wherein the kits comprise at least one primer or probe for a liver-specific polynucleotide. Further disclosed herein are kits for detecting damage to a liver, wherein the kits comprise at least one primer or probe for a liver-specific polynucleotide. Further disclosed herein are kits for detecting damage to a liver, wherein the kits comprise at least three primers or probes for a liver-specific polynucleotide. Further disclosed herein are kits for detecting damage to a liver, wherein the kits comprise at least five primers or probes for a liver-specific polynucleotide. The liver-specific polynucleotide may correspond to a gene selected from the group consisting of: 1810014F10RIK, ABCC2, ABCC6, ABCG5, ACOX2, ACSM2A, ADH1A, ADH1C, ADH6, AFM, AFP, AGXT, AHSG, AKR1C4, AKR1D1, ALB, ALB, ALDH1B1, ALDH4A1, ALDOB, AMBP, ANG, ANGPTL3, AOC3, APCS, APOA1, APOA2, APOB, APOC1, APOC2, APOC3, APOC4, APOE, APOF, APOH, APOM, AQP9, ARID1A, ARSE, ASGR1, ASGR2, ASL, ATF5, C2, C2ORF72, C4A, C4BPA, C6, C8A, C8B, C8G, C9, CAPN5, CES1, CES2, CFHR1, CFHR4, CHD2, CIDEB, CPB2, CPN1, CRLF1, CRYAA, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F11, CYP4F12, CYP4F2, DAK, DCXR, DIO1, DUSP9, F10, F12, F2, F2, FAH, FCN2, FETUB, FMO3, FTCD, G6PC, GABBR1, GALK1, GAMT, GBA, GCKR, GLYAT, GNMT, GPC3, GPT, GSTM1, HAAO, HAMP, HAO1, HGD, HGFAC, HLF, HMGCS2, HP, HPD, HPN, HPR, HPX, HRG, HSD11B1, HSD17B6, IGF2, IGFALS, IGSF1, IL17RB, IL1RN, IQCE, ITIH1, ITIH2, ITIH3, ITIH4, JCLN, KHK, KLK13, LBP, LCAT, LECT2, LGALS4, LOC55908, LPA, MASP2, MAT1A, MGMT, MST1, MSTP9, MUPCDH, NHLH2, NNMT, NR0B2, NR1I2, NSFL1C, OATP1B1, ORM1, PCK1, PEMT, PGC, PKLR, PLG, POLR2C, PON1, PON3, PROC, PXMP2, RBP4, RDH16, RELN, RET, RGN, RHBG, SAA4, SARDH, SDS, SDSL, SEC14L2, SERPINA4, SERPINA5, SERPINA7, SERPINC1, SERPIND1, SERPINF2, SLC10A1, SLC22A1, SLC22A7, SLC27A5, SLC2A2, SLC38A3, SLC6A12, SULT1A2, SULT2A1, TAT, TBX3, TCP10L, TF, TIM2, TMEM176B, TNNI2, TST, UGT2B15, UGT2B17, UPB1, VTN, and WNT7A, and combinations thereof.

Disclosed herein are kits for use in detecting whether or not a tissue or organ is affected by a condition, wherein the kits comprise at least one probe or primer for a marker of the condition. Further disclosed herein are kits for use in detecting the location of a tumor, pathogen or disease, wherein the kits comprise at least one probe or primer for a marker of the condition. In some instances, the kits comprise at least one probe and at least one primer. In some instances, the marker is a polynucleotide and the primer or probe is a polynucleotide that hybridizes to a target of interest. In some instances, the marker is a peptide or protein and the probe is an antibody or antibody fragment capable of binding the peptide or protein. In some instances, the probe is a small molecule that binds to the marker. In some instances, the probe is conjugated to a tag that can be used to retrieve the marker, quantify the marker or detect the marker. The at least one condition or disease may be at least one of: inflammation, apoptosis, necrosis, fibrosis, infection, autoimmune disease, arthritis, liver disease, neurodegenerative disease, and cancer.

Disclosed herein are kits for use in detecting whether or not a tissue or organ is affected by multiple sclerosis, wherein the kits comprise at least one probe or primer for a marker of multiple sclerosis. Disclosed herein are kits for use in detecting whether or not a tissue or organ is affected by multiple sclerosis, wherein the kits comprise at least three probes or primers for a marker of multiple sclerosis. Disclosed herein are kits for use in detecting whether or not a tissue or organ is affected by multiple sclerosis, wherein the kits comprise at least five probes or primers for a marker of multiple sclerosis. The at least one marker may correspond to a gene selected from C3 proactivator, CRP, MBP, MOG, ORM, OPG, PCT, PLP, VCAM-1, ICAM-1, ADAMTS4, BCAS1, CLDN11, CPM CXCL16, EDGE, ELOVL7, ENPP6, ERBB3, EVI2A, FA2H, GAL3ST1, GJA12, GM98, GPR62, GSN, IL23A, MAG, MAL, MMP-9, MOBP, MOG, PLA2G4A, PLEKHH1, PLP1, PLXNB3, PRKCQ, SGK2, SRPK3, TMEM10, TNF-alpha, TRF, TSPAN2, and UGTA8, and combinations thereof. The at least one condition may comprise multiple sclerosis, and the at least one marker may be neopterin,

Disclosed herein are kits for use in detecting whether or not a tissue or organ is affected by inflammation, wherein the kits comprise at least one probe or primer for a marker of inflammation. Disclosed herein are kits for use in detecting whether or not a tissue or organ is affected by inflammation, wherein the kits comprise at least three probes or primers for a marker of inflammation. Disclosed herein are kits for use in detecting whether or not a tissue or organ is affected by inflammation, wherein the kits comprise at least five probes or primers for a marker of inflammation. The marker(s) may correspond to a gene selected from the group consisting of: AHSG, APCS, COX2, FAS, IL6, OPN, ORM1, SIGIRR SOCS3, TFN-alpha, and iNOS, and combinations thereof.

Disclosed herein are kits for use in detecting whether or not a tissue or organ is affected by fibrosis, wherein the kits comprise at least one probe or primer for a marker of fibrosis. Disclosed herein are kits for use in detecting whether or not a tissue or organ is affected by fibrosis, wherein the kits comprise at least three probes or primers for a marker of fibrosis. Disclosed herein are kits for use in detecting whether or not a tissue or organ is affected by fibrosis, wherein the kits comprise at least five probes or primers for a marker of fibrosis. The marker(s) may correspond to a gene selected from the group consisting of: ALT, AST, CO3-610, C06-MMP, C01-764, C4M, CPK, CTGF, IL4, IL6, IL8, IL18, MFAP, MMP1, MMP2, MMP9, MMP13, PDGF, PIIINP, PINP, P4NP 7S, PVCP, TGF-beta, TIMP1, TIMP2, TIMP3, TNF-alpha, and YKL40, and combinations thereof. The condition may be fibrosis, and the at least one marker may be selected from the group consisting of: troponin, type I collagen type II collagen type III collagen type IV collagen, and type V collagen, and combinations thereof. Additional markers of fibrosis may be selected from hyaluronic acid, and various glycoproteins and proteoglycans.

Disclosed herein are kits for use in detecting whether or not a tissue or organ is affected by apoptosis, wherein the kits comprise at least one probe or primer for a marker of apoptosis. Disclosed herein are kits for use in detecting whether or not a tissue or organ is affected by apoptosis, wherein the kits comprise at least three probes or primers for a marker of apoptosis. Disclosed herein are kits for use in detecting whether or not a tissue or organ is affected by apoptosis, wherein the kits comprise at least five probes or primers for a marker of apoptosis. The marker(s) may correspond to a gene selected from the group consisting of: ALB, APOE, CIDEB, F2, PLG, PROC, APAF1, CFLAR, and TNFSF18, and combinations thereof.

Disclosed herein are kits for use in detecting whether or not a subject is affected by a liver disease, wherein the kit comprises at least one marker of a liver disease. Some kits comprise at least three makers of a liver disease. Some kits comprise at least five makers of a liver disease. Liver diseases include, but are not limited to, non-alcoholic fatty liver disease, non-alcoholic steatosis, non-alcoholic steatohepatitis, viral hepatitis, cirrhosis and hepatocarcinoma. Liver diseases may be characterized by the same markers. However, the markers may be present in a sample of the subject at levels that correspond to specific liver diseases. The at least one marker of a liver disease may correspond to a gene selected from the group consisting of: COX2, FAS, IL6, iNOS, LXR-alpha, OPN, PNPLA3 I148M, PPAR-gamma, SOCS3, SREBP-1c, SREBP-2, TFN-alpha, CRP, FIGF, HGF, ICAM1, IL2, IL2RA, IL8RB, KRT18, PI3, REG3A, ST2, TIMP1, TNFR, and TNFRSF1A, and combinations thereof. The kit or system may be useful in determining and/or indicating progress from non-alcoholic fatty liver disease to non-alcoholic steatohepatitis based on detection and analysis steps.

Further disclosed herein are systems for carrying out methods of the present disclosure. In general, a system may comprise various units capable of performing the steps of methods disclosed herein, for example a sample processing unit, an amplification unit, a sequencing unit, a detection unit, a quantifying unit, a comparing unit, and/or a reporting unit. In some embodiments, the system comprises: a memory unit configured to store results of (i) an assay for detecting at least one marker of at least one condition in a first sample of a subject, and (ii) an assay for detecting at least one tissue-specific RNA in a second sample of a subject, wherein the at least one tissue-specific RNA is a cell-free RNA specific to a tissue; at least one processors programmed to: (i) quantify a level of the at least one marker; (ii) quantify a level of the at least one tissue-specific polynucleotide; (iii) compare the level of the at least one marker to a corresponding reference level of the marker; (iv) compare the level of the at least one tissue-specific polynucleotide to a corresponding reference level of the tissue-specific polynucleotide; and (v) determine presence of or relative change in damage of the tissue by the at least one condition based on the comparing; and an output unit that delivers a report to a recipient, wherein the report provides results of step (b). The system may provide a recommendation for medical action based on the results of step (b). The medical action may comprise a treatment. The first sample and the second sample may be the same. The first sample and the second sample may be different. The first sample and the second sample may be different in that they were obtained at different times. The first sample and the second sample may be different in that they are different fluids. The first and/or second sample may be a fluid selected from the group consisting of: blood, a blood fraction, saliva, sputum, urine, semen, a transvaginal fluid, a cerebrospinal fluid, sweat, or a breast fluid. The first and/or second sample may be plasma.

The systems disclosed herein may be used with any one of the kits or devices disclosed herein. The systems may be integrated with any one of the kits or devices disclosed herein. The devices disclosed herein may comprise any one of the systems disclosed herein. In some embodiments, the system comprises a computer system. A computer for use in the system may comprise at least one processor. Processors may be associated with at least one controller, calculation unit, and/or other unit of a computer system, or implanted in firmware as desired. If implemented in software, the routines may be stored in any computer readable memory such as in RAM, ROM, flashes memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in embodiments of the system. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments, the server computer handles all of the database functionality. The client computer can have software that handles all the front-end data management and can also receive data input from users.

Systems disclosed herein may be configured to receive a user request to perform a detection reaction on a sample. The user request may be direct or indirect. Examples of direct request include those transmitted by way of an input device, such as a keyboard, mouse, or touch screen). Examples of indirect requests include transmission via a communication medium, such as over the internet (either wired or wireless).

Systems disclosed herein may further comprise a report generator that sends a report to a recipient, wherein the report contains results of a method described herein. A report may be generated in real-time, such as during a sequencing read or while sequencing data is being analyzed, with periodic updates as the process progresses. In addition, or alternatively, a report may be generated at the conclusion of the analysis. In some embodiments, the report is generated in response to instructions from a user. In addition to the results of detection or comparison, a report may also contain an analysis, conclusion or recommendation based on such results. For example, markers associated with a disease or condition are detected and levels of a tissue-specific polynucleotide are above a normal range, the report may include information concerning this association, such as a likelihood that subject has the disease or condition, which tissues are or are not affected, and optionally a suggestion based on this information (e.g. additional tests, monitoring, or remedial measures). The report can take any of a variety of forms. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. at least one computers, and/or at least one servers).

The disclosure provides a computer-readable medium comprising code that, upon execution by at least one processor, implements a method of the present disclosure. A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computers) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying at least one sequence of at least one instruction to a processor for execution.

### Tissue Specific Polynucleotides

As discussed in the foregoing and following description, methods, systems and kits are provided herein to non-invasively detect a tissue or organ under duress as well as determine which disease or condition is affecting the tissue or organ under duress. Provided herein are kits, devices, systems and methods employing tissue-specific gene expression, tissue-specific nucleic acids (e.g. RNAs) and tissue-specific nucleic acid modifications (e.g., methylation patterns) disclosed herein. The terms, “tissue-specific nucleic acid” and “tissue-specific polynucleotide,” are interchangeable as used herein. The term “tissue-specific” may be used to characterize a nucleic acid that is expressed in a single tissue of the subject. Alternatively, the term “tissue-specific” may be used to characterize a nucleic acid that is predominantly expressed in a specific tissue of the subject. For the purposes of this application, predominantly expressed may mean that the tissue-specific nucleic acid is expressed at an RNA level that is at least 50% greater in the specific tissue than the RNA level of the tissue-specific nucleic acid in any other tissue of the subject. However, in some cases, a tissue-specific nucleic acid expressed at an RNA level that is at least 30% greater in the specific tissue than that of any other tissue may be sufficient for the methods disclosed herein. In other cases, a tissue-specific nucleic acid expressed at an RNA level that is at least 80% greater in the specific tissue than that of any other tissue may be required by the methods disclosed herein. Predominantly expressed may mean that the tissue-specific nucleic acid is expressed at an RNA level that is at least 2-fold greater in the specific tissue of interest than the RNA level of the tissue-specific nucleic acid in any other tissue of the subject. Predominantly expressed may mean that the tissue-specific nucleic acid is expressed at an RNA level that is at least 5-fold greater in the specific tissue of interest than the RNA level of the tissue-specific nucleic acid in any other tissue of the subject. Predominantly expressed may mean that the tissue-specific nucleic acid is expressed at an RNA level that is at least 10-fold greater in the specific tissue of interest than the RNA level of the tissue-specific nucleic acid in any other tissue of the subject. Predominantly expressed may mean that a detectable amount of the tissue-specific nucleic acid would occur in a biological fluid (e.g. plasma) of the subject only when damage occurs to the specific tissue where the tissue-specific nucleic acid is predominantly expressed.

Provided herein are kits, systems and methods for detecting or quantifying a biological molecule in a sample from a subject, including by way of non-limiting example, polynucleotides, peptides/proteins, lipids, and sterols. Biological molecules disclosed herein may be tissue-specific. The term “tissue-specific,” as used herein, generally refers to a biological molecule, or modification thereof, that is expressed at a higher level in the single tissue than in any other tissue in the subject. In some instances, it is expressed at least 10% higher in the single tissue than in any other tissue in the subject. In some instances, it is expressed at least 20% higher in the single tissue than in any other tissue in the subject. In some instances, it is expressed at least 30% higher in the single tissue than in any other tissue in the subject. In some instances, it is expressed at least 40% higher in the single tissue than in any other tissue in the subject. In some instances, it is expressed at least 50% higher in the single tissue than in any other tissue in the subject. Thus, the tissue-specific biological molecule may be considered predominantly present or predominantly expressed in a single tissue. Tissue-specific biological molecules disclosed herein may be tissue-specific polynucleotides. Tissue-specific polynucleotides are nucleic acids that are expressed or modified in a tissue-specific manner. For example, there may be only a single tissue or organ, or small set of tissues or organs that predominantly accounts for the expression of a particular gene (e.g. 60-80%, 90%, 95% or more of a gene's total expression in the subject).

Provided herein are kits, systems and methods for detecting or quantifying a tissue-specific polynucleotide in a sample. At least one database of genetic information can be used to identify a tissue-specific polynucleotide or a panel of tissue-specific polynucleotides. Accordingly, aspects of the disclosure provide systems and methods for the use and development of a database. Methods of the disclosure may utilize databases containing existing data generated across tissue types to identify the tissue-specific genes. Such databases may be utilized for identification of tissue-specific genes. The database may be a web-based gene expression profile. Non-limiting examples of web-based gene expression repositories are publicly available, e.g., The Human Protein Atlas at www.proteinatlas.org, BioGPS at biogps.org and The European Bioinformatics Institute Expression Atlas at www.ebi.ac.uk/gxa/, Gene Expression Omnnibus (GEO) at ncbi.nlm.nih.gov/geo/, the content of all of which are incorporated herein by reference. Such databases are also publicly available as published articles in printed and on-line journals. Databases may also be referred to in the art as atlases, e.g., the Human 133A/GNF1H Gene Atlas (see Su et al., Proc Natl Acad Sci USA, 2004, vol. 101, pp. 6062-7 for original publication) and RNA-Seq Atlas (see Krupp et al., Bioinformatics, 2012, vol. 15, pp. 1184-5 for original publication), which are both incorporated herein by reference. These databases and websites incorporate data from many independent studies and often corroborate tissue specific gene expression patterns amongst a species. Such cross-validation provides useful tissue-specific polynucleotides for methods, systems and kits disclosed herein. In some instances, a tissue-specific polynucleotide disclosed herein is identified as having tissue-specific expression by at least two published datasets. In some instances, a tissue-specific polynucleotide disclosed herein is identified as having tissue-specific expression by at least three published datasets. In some instances, a tissue-specific polynucleotide disclosed herein is identified as having tissue-specific expression by at least four published datasets. In some instances, a tissue-specific polynucleotide disclosed herein is identified as having tissue-specific expression by at least five published datasets. In order to identify tissue-specific transcripts from at least one database, certain embodiments employ a template-matching algorithm to the databases. Template matching algorithms used to filter data are known in the art, see, e.g., Pavlidis P, Noble WS (2001) Analysis of strain and regional variation in gene expression in mouse brain. Genome Biol 2:research0042.1-0042.15. Examples of tissue specific genes include those appearing in FIG. 18 of US20130252835, which is incorporated herein by reference.

Provided herein are kits, systems and methods for detecting or quantifying a tissue-specific polynucleotide in a sample. The tissue-specific nucleic acid may refer to a nucleic acid that is expressed in a single tissue of each subject in a population of subjects. The tissue-specific nucleic acid may refer to a nucleic acid that is predominantly expressed in a specific tissue of each subject in a population of subjects. The population of subjects may be healthy. The population of subjects may have a common disease or condition. The population of subjects may comprise two subjects. The population of subjects may comprise five subjects. The population of subjects may comprise ten subjects. The population of subjects may comprise twenty subjects. The population of subjects may have a common ethnicity, a common genetic background, a common gender, a common age, or a combination thereof. The tissue-specific nucleic acid may refer to a nucleic acid that is expressed in a single tissue or predominantly expressed in a specific tissue as shown by a published study or database. The published study may have employed microarray technology or RNA-seq profiling to measure tissue specific nucleic acid levels. In some instances, damage of the specific tissue is caused by a disease or condition resulting in apoptosis of cells in the specific tissue, releasing cell-free tissue-specific nucleic acids into a circulating fluid of the subject. The tissue-specific nucleic acid may be a nucleic acid that is expressed highly enough in the specific tissue that it can be detected in a circulating biological fluid (e.g. blood, plasma) when damage to the specific tissue occurs. The tissue-specific nucleic acid may be a nucleic acid that is expressed highly enough in the specific tissue that it can be detected in a circulating biological fluid (e.g. blood, plasma) when damage to at least about 10%, at least about 20%, at least about 30%, at least about 40% or at least about 50% of the specific tissue occurs.

Disclosed herein are methods, kits and systems for detecting, quantifying, and/or analyzing tissue-specific polynucleotides. In general, the tissue-specific polynucleotides are cell-free polynucleotides, released into a biological fluid (e.g. blood, cerebrospinal fluid, lymphatic fluid, urine), upon damage or injury to a cell, tissue or organ. As used herein, damage or injury to the cell, tissue or organ may be due to a disease or condition that results in disruption of a cell membrane or a loss of cell membrane integrity of the cell or at least one cell within or on the surface of the tissue or organ. Disruption of the cell membrane or loss of cell membrane integrity may result in a release of polynucleotides within the cell. Disruption of the cell membrane may be due, for instance to necrosis, autolysis, or apoptosis. Non-limiting examples of tissue-specific polynucleotides include tissue-specific RNA, and DNA comprising a tissue-specific methylation pattern. Tissue-specific RNAs may include, but are not limited to, messenger RNA (mRNA), a microRNA (miRNA), a pre-miRNA, a pri-miRNA, a pre-mRNA, a circular RNA (circRNA), a long non-coding RNA (lncRNA), and an exosomal RNA. Examples of genes having tissue-specific expression are provided herein.

Provided herein are kits, systems and methods for detecting or quantifying a biological molecule in a sample from a subject. Biological molecules disclosed herein may be tissue-specific. The term “tissue-specific,” as used herein, generally refers to a biological molecule, or modification thereof, that is expressed at a higher level in the single tissue than in any other tissue in the subject. In some instances, it is expressed at least 10% higher in the single tissue than in any other tissue in the subject. In some instances, it is expressed at least 20% higher in the single tissue than in any other tissue in the subject. In some instances, it is expressed at least 30% higher in the single tissue than in any other tissue in the subject. In some instances, it is expressed at least 40% higher in the single tissue than in any other tissue in the subject. In some instances, it is expressed at least 50% higher in the single tissue than in any other tissue in the subject. Thus, the tissue-specific biological molecule may be considered predominantly present or predominantly expressed in a single tissue. Tissue-specific biological molecules disclosed herein may be tissue-specific polynucleotides. Tissue-specific polynucleotides are nucleic acids that are expressed or modified in a tissue-specific manner. For example, there may be only a single tissue or organ, or small set of tissues or organs that predominantly accounts for the expression of a particular gene (e.g. 60-80%, 90%, 95% or more of a gene's total expression in the subject).

In some instances, methods disclosed herein comprise comparing the level of a single tissue-specific polynucleotide to a corresponding reference level of the tissue-specific polynucleotide is sufficient to determine whether a tissue has been damaged by a disease or condition. In other instances, the level of multiple tissue-specific polynucleotides may be compared to corresponding reference levels of the tissue-specific polynucleotides to determine whether a tissue has been damaged by a disease or condition. The methods disclosed herein may comprise comparing the level of as few as 1, 2, 3, 4, 5, 6, 7, 8, 9 or 10 tissue-specific polynucleotides to corresponding reference levels to determine whether a tissue that has been damaged by a disease or condition. There may be an advantage to comparing as few as 1, 2 or 3 tissue-specific polynucleotides to corresponding reference levels.

In some instances, methods disclosed herein comparing the level of a tissue-specific polynucleotide to a corresponding reference level of the tissue-specific polynucleotide results in determining that the level of the tissue-specific polynucleotide is greater than the corresponding reference level. In some cases, the corresponding reference level is the level of the tissue-specific polynucleotide in a healthy individual and the level of the tissue-specific polynucleotide being greater than the corresponding reference level is indicative of damage or injury to a specific tissue, organ or cell in the subject. The level of the tissue-specific polynucleotide may be at least about 5%, at least about 10%, at least about 20%, at least about 30%, at least about 40%, at least about 50%, at least about 60%, at least about 70%, at least about 80%, at least about 90%, at least about 100%, at least about 150%, or at least about 200% greater than the corresponding reference level.

In some instances, methods disclosed herein comparing the level of a tissue-specific polynucleotide to a corresponding reference level of the tissue-specific polynucleotide results in determining that the level of the tissue-specific polynucleotide is lower than the corresponding reference level. In some cases, the corresponding reference level is the level of the tissue-specific polynucleotide in an individual or population having the disease or condition, and the level of the tissue-specific polynucleotide being lower than the corresponding reference level is indicative of the absence or minimal amount of damage or injury to a specific tissue, organ or cell in the subject. The level of the tissue-specific polynucleotide may be at least about 5%, at least about 10%, at least about 20%, at least about 30%, at least about 40%, at least about 50%, at least about 60%, at least about 70%, at least about 80%, at least about 90%, or at least about 95% lower than the corresponding reference level.

Tissue-specific polynucleotides disclosed herein may be described as “corresponding to a gene.” In some instances, the phrase “corresponding to a gene” means the tissue-specific polynucleotide is transcribed from a gene. Thus, in some instances, tissue-specific polynucleotides are tissue-specific RNA transcripts. Tissue-specific RNA transcripts include full-length transcripts, transcript fragments, transcript splice variants, enzymatically or chemically cleaved transcripts, transcripts from two or more fused genes, and transcripts from mutated genes. Fragments and cleaved transcripts must retain enough of the full-length polynucleotide to be recognizable as correspond to the gene. In some instances, 5% of the full-length polynucleotide is enough of the full-length polynucleotide. In some instances, 10% of the full-length polynucleotide is enough of the full-length polynucleotide. In some instances, 15% of the full-length polynucleotide is enough of the full-length polynucleotide. In some instances, 20% of the full-length polynucleotide is enough of the full-length polynucleotide. In some instances, 25% of the full-length polynucleotide is enough of the full-length polynucleotide. In some instances, 30% of the full-length polynucleotide is enough of the full-length polynucleotide. In some instances, 40% of the full-length polynucleotide is enough of the full-length polynucleotide. In some instances, 50% of the full-length polynucleotide is enough of the full-length polynucleotide. In some instances, the phrase “corresponding to a gene” means the tissue-specific polynucleotide is a modified form of the gene (e.g., tissue-specific DNA modification pattern).

Methods, systems and kits disclosed herein may use tissue-specific polynucleotides to identify damage or injury to the liver that correspond to a gene selected from a group including, but not limited to, 1810014F10RIK, A1BG, ABCC2, ABCC6, ABCG5, ANG, ANGPTL3, ACOX2, ACSM2A, ADH1A, ADH1C, ADH6, AFM, AFP, AGXT, AHSG, AKR1C4, AKR1D1, ALB, ALDH1B1, ALDH4A1, ALDOB, AMBP, AOC3, APCS, APOA1, APOA2, APOA5, APOB, APOC1, APOC2, APOC3, APOC4, APOE, APOF, APOH, APOM, ARID1A, ARSE, ASL, AQP9, ASGR1, ASGR2, ATF5, C4A, C4BPA, C6, C8A, C8B, C8G, C9, CAPN5, CES1, CES2, CFHR1, CFHR2, CFHR3, CFHR4, CFHR5, CHD2, CIDEB, CPN1, CRLF1, CRYAA, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F12, DIO1, DAK, DCXR, F10, F12, F2, F9, FAH, FCN2, FETUB, FGA, FGB, FGG, FMO3, FTCD, G6PC, GPC3, GALK1, GAMT, GBA, GBP7, GCKR, GLYAT, GNMT, GPT, GSTM1, HAAO, HAMP, HAO1, HGD, HGFAC, HMGCS2, haptoglobin, HPN, HPR, HPX, HRG, HSD11B1, HSD17B6, HLF, IGF2, IL1RN, IGFALS, IQCE, ITIH1, ITIH2, ITIH4, JCLN, KHK, KLK13, LBP, LECT2, LOC55908, LPA, MASP2, MBL2, MGMT, MUPCDH, NHLH2, NNMT, NSFL1C, OATP1B1, ORM2, PCK1, PEMT, PGC, PLG, PKLR, PLGLB2, POLR2C, PON1, PON3, PROC, PXMP2, RBP4, RDH16, RET, SAA4, SARDH, SDS, SDSL, SEC14L2, SERPINA4, SERPINA7, SERPINA10, SERPINA11, SERPINC1, SERPIND1, SLCO1B1, SLC10A1, SLC22A1, SLC22A7, SLC22A10, SLC25A47, SLC27A5, SLC38A3, SLC6A12, SPP2, TAT, TBX3, TF, TIM2, TMEM176B, TST, UPB1, UROC1, VTN, WNT7A, C2, C2ORF72, CPB2, CYP4F11, CYP4F2, DUSP9, GABBR1, HP, HPD, IGSF1, IL17RB, ITIH2, ITIH3, LCAT, LGALS4, MAT1A, MST1, MSTP9, NR0B2, NR1I2, ORM1, RELN, RGN, RHBG, SAA4, SERPINA5, SERPINA7, SERPINC1, SERPINF2, SLC2A2, SULT1A2, SULT2A1, TCP10L, TNNI2, UGT2B15, and UGT2B17, and combinations thereof. The tissue-specific polynucleotide may be used to identify damage or injury to at least one distinct cell type in the liver. The tissue-specific polynucleotide may be used to identify damage or injury to at least one distinct cell type in the liver because expression of the tissue-specific polynucleotide differs between the at least one distinct cell type and the remaining cells of the liver. By way of non-limiting example, the at least one distinct cell type may be selected from a hepatic stellate cell, a Kupffer cell, a sinusoidal endothelial cell, and a hepatocyte.

Methods, systems and kits disclosed herein may use tissue-specific polynucleotides to identify damage or injury to the kidney that correspond to a gene selected from a group including, but not limited to, AK3L1, AQP2, AQPN6, ATP6V1G3, ATP6V0D2, BBOX1, BFSP2, BHMT, BSND, C20ORF194, C9orf66, CALB1, CA12, CDH16, CLCNKA, CRYAA, CRYBB3, CTXN3, CUBN, CYS1, DDC, DNMT3L, EGF, ENPEP, FCAMR, FMO1, FOLR3, FUT3, FXYD2, FXYD4, GGT1, HAO2, HAVCR1, HKID, HMX2, HNF1B, KAAG1, KCNJ1, KL, MCCD1, MIOX, NAT8, NOX4, NPHS2, OR2T10, PAX2, PDZK1, PDZK1IP1, PRR35, PTH1R, RBP5, SIM1, SLC12A1, SLC12A3, SLC13A3, SLC17A3, SLC22A11, SLC22A12, SLC22A13, SLC22A2, SLC22A24, SLC22A6, SLC22A8, SLC22A13, SLC34A1, SLC3A1, SLC4A9, SLC5A2, SLC5A10, SLC6A13, SLC6A18, SLC7A7, SLC7A8, SLC7A9, SOST, TREH, TMEM27, TMEM52B, TMEM72, TMEM174, TMEM207, UGT1A1, UGT1A6, UGT1A9, UMOD, UPP2, XPNPEP2, 0001T8, and combinations thereof. The tissue-specific polynucleotide may be used to identify damage or injury to at least one distinct cell type in the kidney. The tissue-specific polynucleotide may be used to identify damage or injury to at least one distinct cell type in the kidney because expression of the tissue-specific polynucleotide differs between the at least one distinct cell types and the remaining cells of the kidney. By way of non-limiting example, the at least one distinct cell type may be selected from kidney glomerulus parietal cells, kidney glomerulus podocytes, kidney proximal tubule brush border cells, loop of Henle thin segment cells, thick ascending limb cells, kidney distal tubule cells, collecting duct principal cells, collecting duct intercalated cells, interstitial kidney cells, and combinations thereof.

Methods, systems and kits disclosed herein may use tissue-specific polynucleotides to identify damage or injury to the heart and/or cardiovascular system that correspond to a gene selected from a group including, but not limited to, ACTC1, ANKRD1, ASB18, BMP10, CASQ2, CCDC141, CHRNE, CORIN, CSRP3, DAND5, FABP3, GJA3, KLHL31, LRRC10, MT1HL1, MYBPC3, MYBPHL, MYH6, MYH7, MYL2, MYL3, MYL4, MYL7, MYOZ2, MYZAP, NPPA, NPPB, PLN, POPDC2, PPP1R1C, PRSS42, RD3L, RMB20, RYR2, SBK2, SBK3, SCN5A, SMCO1, ST8SIA2, TBX20 TECRL, TNNI3, TNNI3K, TNNT2, and XIRP1, and combinations thereof. Tissue-specific polynucleotides that may be used to identify damage or injury to the coronary artery may correspond to a gene selected from a group including, but not limited to, CNTN4, CASQ2, MYOCD, FHL5, NPR3, ACADL, FIBIN, MRAP2, CNN1, SLC22A3, SEMA3D, NPR1, NPNT, PLN, SBSPON, C7, and FPR3, and combinations thereof. The tissue-specific polynucleotide may be used to identify damage or injury to at least one distinct cell type of the heart and/or cardiovascular system. The tissue-specific polynucleotide may be used to identify damage or injury to at least one distinct cell type in the heart and/or cardiovascular system because expression of the tissue-specific polynucleotide differs between the at least one distinct cell type and the remaining cells of the heart and/or cardiovascular system. By way of non-limiting example, the at least one distinct cell type may be selected from a cardiomyocytes, vascular endothelial cells, endocardial endothelial cells, endothelial progenitor cells, vascular smooth muscle cells, resident vascular leukocytes, and cardiac fibroblasts, and combinations thereof.

Methods, systems and kits disclosed herein may use tissue-specific polynucleotides to identify damage or injury to the breast, uterus, or ovary that correspond to a gene selected from a group including, but not limited to, ANGPTL5, ARX, C/EBP-delta, CRYGD, ECEL1, GRO-alpha, GRO-beta, HIN-1, IK-alpha, IL-8, KLHDC8A, LIF, M1S1, MIP3-alpha, MMP10, MMP26, MUM1L1, PRP, RASD1, RP4-559A3.7, RPS6, SOD2, TM4SF1, TNFAIP2 TRH, and WFIKKN2, and combinations thereof. The tissue-specific polynucleotide may be used to identify damage or injury to at least one distinct cell type of the breast, ovary, and uterus. The tissue-specific polynucleotide may be used to identify damage or injury to at least one distinct cell type in the breast, ovary, and uterus because expression of the tissue-specific polynucleotide differs between the at least one distinct cell type and the remaining cells of the breast, ovary, and uterus. By way of non-limiting example, the at least one distinct cell type may be selected from follicular cells, granulosa cells, mammary epithelial cells, myoepithelial cells, luminal epithelial cells, adipocyte, mast cell, and endometrial cells, and combinations thereof.

Methods, systems and kits disclosed herein may use tissue-specific polynucleotides to the brain or nervous system that correspond to a gene selected from a group including, but not limited to, ADAMTS4, AMER2, BCAS1, CRP, C1orf61, C2orf80, C3 proactivator, C8orf46, CACNG7, CACNG8, CAMKV, CLDN11, CPM, CREG2, CSPG5, CXCL16, DLL3, EDGE, ELAVL3, ELOVL7, ENPP6, ERBB3, ERMN, EVI2A, FA2H, FEZF2, GABRA1, GAL3ST1, GFAP, GJA12, GM98, GPR37L1, GPR62, GRIN1, GRM3, GSN, HPOA, IL23A, KCNJ9, LRTM2, MAG, MAL, MMP-9, MOBP, MOG, MOG, MBP, MOG, OPG, NCAN, NEUROD2, NEUROD6, NR2E1, OLIG1, OLIG2, OMG, ORM, OPALIN, PCDHGC5, PLA2G4A, PLEKHH1, PLP1, PLXNB3, PMP2, POU3F2, PRKCQ, PCT, PLP, PSD2 RASL10A, RGR, SEZ6, SGK2, SLC12A5, SLC17A6, SLC17A7, SLC39A12, SLITRK1, SNCB, ICAM-1, VCAM-1, SRPK3, TBR1, TMEM10, TMEM235, TNF-alpha, TRF, TSPAN2, TTC9B, UGTA8, VSTM2B, and ZDHHC22, and combinations thereof. The tissue-specific polynucleotide may be used to identify damage or injury to at least one distinct cell type of the brain and/or nervous system. The tissue-specific polynucleotide may be used to identify damage or injury to at least one distinct cell type in the brain and/or nervous system because expression of the tissue-specific polynucleotide differs between the at least one distinct cell type and the remaining cells of the brain and/or nervous system. By way of non-limiting example, the at least one distinct cell type may be selected from glial cells and neurons. The glial cells may be selected from astrocytes, microglia, and oligodendrocytes, Schwann cells, and combinations thereof.

Methods, systems and kits disclosed herein may use tissue-specific polynucleotides to identify damage or injury to the pancreas that correspond to a gene selected from a group including, but not limited to, AMY2A, AMY2B, AQP12A, AQP12B, CEL, CELA2A, CELA2B, CELA3A, CELA3B, CLPS, CLPSL1, CPA1, CPA2, CPB1, CTRB1, CTRB2, CTRC, CTRL, G6PC2, GP2, LAPP, Insulin, KIRREL2, PDIA2, PLA2G1B, PM20D1, PNLIP, PNLIPRP1, PPY PRSS1, PRSS3, PRSS3P2, PTF1A, RBPJL, SERPINI2, SPINK1, and SYCN, and combinations thereof. The tissue-specific polynucleotide may be used to identify damage or injury to at least one distinct cell type of the pancreas. The tissue-specific polynucleotide may be used to identify damage or injury to at least one distinct cell type in the pancreas because expression of the tissue-specific polynucleotide differs between the at least one distinct cell type and the remaining cells of the pancreas. By way of non-limiting example, the at least one distinct cell type may be selected from acinar cells, Langerhans cells, columnar cells, ductal cells, and combinations thereof. Langerhans cells may be selected from alpha cells, beta cells, delta cells, PP cells, and epsilon cells, and combinations thereof.

Methods, systems and kits disclosed herein may use tissue-specific polynucleotides to identify damage or injury to the colon, stomach or gastrointestinal system that correspond to a gene selected from a group including, but not limited to, A4GNT, AC009133.22, ADA, ADORA2B, ADTRP, AIFM3, ANXA10, ATP2C2, ATP4A, ATP4B, B3GALT1, B3GALT5, B3GNT3, B3GNT6, B3GNT7, B4GALNT2, BARX1, C15orf48, C2orf72, C9orf152, CA2, CA4, CALML4, CAPN8, CCL1, CCL15, CD70, CDC42EP5, CEA, CEACAM1, CEACAM5, CES3, CKMT1B, CLDN23, CLDN3, CPA1, CPA2, CPA6, DAZ2, DAZ3, DAZ4, DHRS9, DPCR1, ENTPD8, EPCAM, ERN2, FABP6, FAM101A, FAM3D, FAM83E, FAM84A, FOXD2, FOXQ1, FRMD1, FUT3, FXYD3, GALNT5, GAST, GHRL, GIF, GJD3, GKN1, GKN2, GUCA2B, HAVCR1, HMGCS2, HOXB9, HOXD11, HOXD12, ITLN1, KCNE2, KCNJ13, KLK15, KRT20 LIPF, MLN, MS4A18, MUC5AC, NAALADL1, O, ONECUT3, PDX1, PGA3, PGA4, PGA5, PGC, PLB1, PSCA, S100G, SLC17A8, SLC9A4, TAAR1, TFF1, TFF2, and TMPRSS15, and combinations thereof. Tissue-specific polynucleotides that may be used to identify damage or injury to the esophagus may correspond to a gene selected from a group including, but not limited to, A2ML1, ADH7, CAPN14, CRABP2, CRNN, CSTB, DEFB104A, DEFB104B, DYNAP, ECM1, EPGN, FABP5, FAM83A, FGFBP1, GBP6, GJB2, IGFL1, KLK13, KRT13, KRT32, KRT4, KRT6A, KRT6B, KRT6C, KRT78, KRTAP3-2, MAL, MUC21, MUC22, PADI 1, PRSS27, RAET1L, RHCG, SCGB2A2, SERPINB13, SERPINB3, SPRR1A, SPRR1B, SPRR2A, SPRR2B, SPRR2D, SPRR3, TGM1, TGM3, TMPRSS11E, UGT1A7, ZNF185, and ZNF812, and combinations thereof. The tissue-specific polynucleotide may be used to identify damage or injury to at least one distinct cell type of the colon, stomach or gastrointestinal system. The tissue-specific polynucleotide may be used to identify damage or injury to at least one distinct cell type in the colon, stomach or gastrointestinal system because expression of the tissue-specific polynucleotide differs between the at least one distinct cell type and the remaining cells of the colon, stomach or gastrointestinal system. By way of non-limiting example, the at least one distinct cell type may be selected from stem cells, paneth cells, goblet cells, enterocytes, colonocytes, plasma cells, mesenchymal cells, enteroendocrine cells, parietal cells, chief cells, and columnar epithelial cells, and combinations thereof.

Methods, systems and kits disclosed herein may use tissue-specific polynucleotides to identify damage or injury to the lung that correspond to a gene selected from a group including, but not limited to, SFTPC, SFTPA1, SFTPB, SFTPA2, AGER, SCGB3A2, SFTPD, ROS1, MS4A15, RTKN2, NAPSA, LRRN4, SCGB1A1, SLC34A2, CACNA2D2, SFTA2, LAMP3, SLC22A31, DCSTAMP, and WIFE, and combinations thereof. The tissue-specific polynucleotide may be used to identify damage or injury to at least one distinct cell type of the lung. The tissue-specific polynucleotide may be used to identify damage or injury to at least one distinct cell type in the lung because expression of the tissue-specific polynucleotide differs between the at least one distinct cell type and the remaining cells of the lung. By way of non-limiting example, the at least one distinct cell type may be selected from alveolar type I epithelial cells, alveolar type II epithelial cells, capillary endothelial cells, alveolar macrophages, and combinations thereof.

Methods, systems and kits disclosed herein may use tissue-specific polynucleotides to identify damage or injury to the skin that correspond to a gene selected from a group including, but not limited to, AADACL2, ABCA12, ABHD12B, AHNAK2, ALOXE3, ASPRV1, BPIFC, C1orf68, CARD18, CASP14, CCL27, CDSN, CDX4, CLEC2A, COL17A1, COL7A1, CST6, DCT, DGAT2L6, DMKN, DNASE1L2, DSC1, DSG1, FAM2BP, FGFR3, FLG, FLG2, GAN, GJB4, GSDMA, HOXC13, HS3ST6, IGFL3, IGFL4, IL37, KCNK7, KLK14, KLK5, KPRP, KREMEN2, KRT1, KRT10, KRT14, KRT2, KRT73, KRT77, KRT79, KRTDAP, LAMB4, LCE1A, LCE1B, LCE1C, LCE1D, LCE1E, LCE1F, LCE2A, LCE2B, LCE2C, LCE2D, LCE4A, LCE5A, LCE6A, LGALS7B, LGASLS7, LIPK, LIPM, LIPN, LOR, LY6G6C, MLANA, NEU2, NKPD1, NLRP10, PLA2G4E, PLA2G4F, PMEL, POU2F3, POU3F1, PSORS1C2, PYDC1, SERPINA12, SLC24A5, SPRR2G, SPRRR4, THEW5, TMEM45A, TREX2, TYR, UCN2, WFDC12, WFDC5, WNT16, and WNT3, and combinations thereof.

Methods, systems and kits disclosed herein may use tissue-specific polynucleotides to identify damage or injury to the thyroid gland that correspond to a gene selected from a group including, but not limited to, BMP8A, CALCA, CALCB, CRYGN, DIO2, FKSG66, FKSG66, FOXE1, GRK1, IGFBPL1, INPP5J, IYD, KIAA1456, KRT83, LIPI, OTOS, PAX8, PDE8B, RAG2, RGS16 SCUBE3, SLC26A4, SLC26A7, SLC5A8, TCERG1L, TG, TPO, and TSHR, and combinations thereof.

Methods, systems and kits disclosed herein may use tissue-specific polynucleotides to identify damage or injury to the prostate that correspond to a gene selected from a group including, but not limited to, ACPP, CHRNA2, COL9A1, KLK2, KLK3, KLK4, MSMB, NCAPD3, NEFH, NKX3-1, OR51E2, RDH11, RFPL2, RLN1, SLC30A4, SLC45A3, SP8, STEAP2, TBL1Y, TGM4, and TRPM8, and combinations thereof.

Methods, systems and kits disclosed herein may use tissue-specific polynucleotides to identify damage or injury to the testis that correspond to a gene selected from a group including, but not limited to, ACSBG2, ACTL7A, ACTRT2, ADAD1, AKAP4, ANKRD7, BOD1L2, C10orf62, C2or57, C5orf58, CCDC70, CETN1, CMTM2, CST9L, DEFB119, DEFB123, FATE1 FMR1NB, FNDC8, GK2, H1FN %, HDGFL1, IQCF1, IRGC, KOF2B, LELP1, LYPD4, ODF1, PDHA2, PGK2, PRM1, PRM2, PRR30, RP11-322n21.2, SEPT12, SHCBP1L, SLC25A31, SMCP, SPACA7, SPATA16, SPATA3, SPATA8, TEX38, TMCO2, TNP1, TPD52L3, TSACC, TUBA3C, UBQLN3, and ZPBP, and combinations thereof.

Methods, systems and kits disclosed herein may use tissue-specific polynucleotides to identify damage or injury to the urinary bladder or gallbladder that correspond to a gene selected from a group including, but not limited to, AC233755.1, CAPN12, CHST4, DHRS2, FGF19, MMP13, MOGAT1, MUC5B, RP11-1012A1.4, SNX31, UGT2B11, UGT2B28, and UPK2, and combinations thereof.

Methods, systems and kits disclosed herein may use tissue-specific polynucleotides to identify damage or injury to the bone marrow or disruption of cell membranes of bone marrow cells that correspond to a gene selected from a group including, but not limited to, ABCA13, AHSP, ALAS2,AZU1, BPI, CAMP, CCL3L3, CEACAM8, CEBPE, CTSG, DEFA1, DEFA1B, DEFA3, DEFA4, ELANE, EPB42, EPX, FGAR, GATA1, GFI1B, GYPA, GYPB, HBB, HBD, HBM, HIST1H1E, HIST1H2AL, IFIT1B, KLF1, MMP8, MPO, MS4A3, PAD14, PGLYRP1, PRG3, PRSS57, PRTN3, RAB44, RHAG, RHCE, RHD, RNA SE2, RNA SE3, S100A12, SERPINB10, SPTA1, TARM1, TSPO2, VPREB1, and VSTM1, and combinations thereof.

Methods, systems and kits disclosed herein may use tissue-specific polynucleotides to the retina that correspond to a gene selected from a group including, but not limited to, RBP3, OPTC, RHO, RPE65, RLBP1, GNAT1, OTX2, RCVRN, RGR, PPEF2, PDC, SIX3, PDE6G, CRYBA1, RGR, ARR3, IMPG1, NRL, PDE6A, SAG, LRAT, AIPL1, GUCA1A, GNGT1, and GRIM, and combinations thereof.

Aging is often associated with a decrease in subcutaneous fat, skin thinning and wrinkles, as well as an increase in deposition of visceral fat, which is associated with cardiometabolic syndromes and risk of cardiovascular diseases. Tissue-specific polynucleotides associated with subcutaneous fat are encoded by genes including, but not limited to, SHOX, HTRA4, C10orf142, ANGPTL5, SIM1, EGFL6, HOXC12, MMP27, TBX15, OPN4, FAM180B, SHOX2, and EN1, and combinations thereof. Tissue-specific polynucleotides associated with visceral fat include are encoded by genes including, but not limited to, HAS1, FOSB, ITLN1, IL6, C1QTNF9, FFAR3, ALOX15, CCL8, SOCS3, NWD2, OR51E1, SELE, RP11-903H12.5, CSF3, CRYBB1, EGR1, CH25H, ADGRG2, LRRN4, FOS, BARX1, IL2RA, CD200R1, CXCL8, GDF6, TNFSF14, RARRES1, and IDO1, and combinations thereof.

### Markers of a Disease or Condition

As discussed in the foregoing and following description, methods, systems and kits are provided herein to non-invasively detect a tissue or organ under duress as well as determine which disease or condition is affecting the tissue or organ under duress. Disclosed herein are methods, kits and systems for detecting, quantifying, and/or analyzing at least one marker of a disease or condition. Similar to the tissue-specific polynucleotides disclosed herein, a marker may be a cell-free polynucleotide, released into a biological fluid (e.g. blood, cerebrospinal fluid, lymphatic fluid, urine), upon damage or injury to a tissue or organ. In some cases, the at least one marker of the disease or condition comprises a tissue-specific polynucleotide disclosed herein. Damage or injury to the tissue or organ may be due to a disease or condition that results in cytolysis within or on the surface of the tissue or organ.

Markers disclosed herein, by way of non-limiting example, may be selected from a peptide, a protein, an aptamer, an antibody, a cell fragment, a sterol (e.g., cholesterol), a hormone, a lipid, a phospholipid, a fatty acid, a sugar moiety, a vitamin, a metabolite, and an extracellular matrix component, complexes thereof, and chemical modifications thereof. Chemical modifications may include, but are not limited to, phosphorylation, myristoylation, palmitoylation, acetylation, methylation, sumoylation, glycosylation and ubiquitination. The methods disclosed herein may comprise an assay to detect these markers. A variety of suitable assays are available, selection of which may depend on the type of marker to be detected. By way of non-limiting example, these assays include ELISA, Western blot, gel electrophoresis, and reporter assays. Any suitable number of markers for any or more diseases or conditions may be assayed in parallel or in a single reaction. For example, an assay may comprise detecting 5, 10, 25, 50, 75, 100, 250, 500, 1000, or more markers, for the assessment of 1, 2, 3, 4, 5, 10, 15, 25, or more diseases or conditions. Any convenient assay format for such multiplexed reactions may be employed, examples of which are provided herein, including but not limited to microarray analysis and high-throughput sequencing methodologies.

Alternatively or additionally, a marker may comprise a cell count of at least one cell type. By way of non-limiting example, a platelet count of less than about 150,000 along with cell-free liver-specific polynucleotides may indicate the subject suffers hepatitis and that the hepatitis may be stage 3 or stage 4 hepatitis.

Disclosed herein are methods, kits and systems for detecting, quantifying, and/or analyzing at least one marker of a disease or condition, wherein the marker is a cell-free polynucleotide. Non-limiting examples of cell-free polynucleotides as markers include RNA and DNA (including DNA comprising a tissue-specific methylation pattern). Examples of RNA useful as a marker for a disease or condition include, but are not limited to, messenger RNA (mRNA), microRNA (miRNA), pre-miRNA, pri-miRNA, pre-mRNA, eukaryotic RNA, prokaryotic RNA, viral RNA, bacterial RNA, parasitic RNA, fungal RNA, viroid RNA, virusoid RNA, circular RNA (circRNA), ribosomal RNA (rRNA), transfer RNA (tRNA), pre-tRNA, long non-coding RNA (lncRNA), small nuclear RNA (snRNA), and exosomal RNA. DNA may include single stranded DNA, double stranded DNA, DNA-protein complexes, mitochondrial DNA, bacterial DNA and DNA with specific chemical modification patterns (e.g., methylated DNA). Bacterial DNA/RNA may include those of gut organisms and may be markers of a dietary sensitivity, gut condition or metabolic condition.

The presence, or relative or absolute quantity of the at least one marker in a subject's sample may be indicative of the presence, stage, or progression of a disease or condition, a response to a therapy administered to the subject to treat the disease or condition, or indicative of how a subject might respond to a particular treatment. In some cases, a lower level of the at least one marker in the sample relative to a reference level may be indicative of the presence, stage, or progression of a disease or condition, or a response to a therapy administered to the subject to treat the disease or condition. In some cases, a higher level of the at least one marker in the sample relative to a reference level may be indicative of the presence, stage, or progression of a disease or condition, or a response to a therapy administered to the subject to treat the disease or condition. A mutation or specific sequence of the at least one marker may be indicative of the presence, stage, or progression of a disease or condition, or a response to a therapy administered to the subject to treat the disease or condition. The quantity of the at least one marker with a specific mutation or sequence may be indicative of the presence, stage, or progression of a disease or condition, or a response to a therapy administered to the subject to treat the disease or condition.

Markers disclosed herein may be described as “corresponding to a gene.” In some instances, the phrase “corresponding to a gene” means the marker is transcribed from a gene. Thus, in some instances, a marker is a RNA transcript. RNA transcripts include full-length transcripts, transcript fragments, transcript splice variants, enzymatically or chemically cleaved transcripts, transcripts from two or more fused genes, and transcripts from mutated genes. Fragments and cleaved transcripts must retain enough of the full-length polynucleotide to be recognizable as corresponding to the gene. In some instances, 5% of the full-length polynucleotide is enough of the full-length polynucleotide. In some instances, 10% of the full-length polynucleotide is enough of the full-length polynucleotide. In some instances, 15% of the full-length polynucleotide is enough of the full-length polynucleotide. In some instances, 20% of the full-length polynucleotide is enough of the full-length polynucleotide. In some instances, 25% of the full-length polynucleotide is enough of the full-length polynucleotide. In some instances, 30% of the full-length polynucleotide is enough of the full-length polynucleotide. In some instances, 40% of the full-length polynucleotide is enough of the full-length polynucleotide. In some instances, 50% of the full-length polynucleotide is enough of the full-length polynucleotide. In some instances, the phrase “corresponding to a gene” means the tissue-specific polynucleotide is a modified form of the gene (e.g., tissue-specific DNA modification pattern). In some instances, the phrase “corresponding to a gene” means the marker is a protein encoded by a gene. The protein may be a full-length protein, a cleaved protein, a protein fragment, a pro-form of a protein (e.g., before naturally occurring enzymatic cleavage), an insoluble version of the protein, a soluble protein, a secreted protein, a protein that is released from a cell upon cell death, or a protein that is released from a tissue upon tissue damage. Fragments and cleaved proteins must retain enough of the full-length protein to be recognizable as corresponding to the gene. In some instances, 5% of the full-length protein is enough of the full-length protein. In some instances, 10% of the full-length protein is enough of the full-length protein. In some instances, 15% of the full-length protein is enough of the full-length protein. In some instances, 20% of the full-length protein is enough of the full-length protein. In some instances, 25% of the full-length protein is enough of the full-length protein. In some instances, 30% of the full-length protein is enough of the full-length protein. In some instances, 40% of the full-length protein is enough of the full-length protein. In some instances, 50% of the full-length protein is enough of the full-length protein.

Disclosed herein are methods, kits and systems for detecting, quantifying, and/or analyzing at least one cancer marker. A cancer marker may comprise a mutation in a polynucleotide or peptide corresponding to a gene selected from, but not limited to, a gene in Table 1, and mutants thereof.

TABLE 1
Genes with Cancer Markers

ABI1, ABL1, ABL2, ACKR3, ACSL3, ACSL6, ACVR1B, AFF1, AFF3, AFF4, AFRP1, AKAP9,
AKT1, AKT2, AKT3, ALDH2, ALK, AMER1, APC, AR, ARAF, ARHGAP26, ARHGEF12,
ARID1A, ARID1B, ARID2, ARNT, ASPSCR1, ASXL1, ATF1, ATIC, ATM, ATP1A1, ATP2B3,
ATR, ATRX, AURKA, AURKB, AXIN1, AXL, BAP1, BARD1, BCL10, BCL11A, BCL11B, BCL2,
BCL2L1, BCL2L2, BCL3, BCL6, BCL7A, BCL9, BCOR, BCORL1, BCR, BIRC3, BLM,
BMPR1A, BRAF, BRCA1, BRCA2, BRD3, BRD4, BRIP1, BTG1, BTK, BUB1B, C11orf30,
C15orf65, C2orf44, CACNA1D, CALR, CAMTA1, CANT1, CARD11, CARS, CASC5, CASP8,
CBFA2Te, CBFB, CB L, CBLB, CBLC, CCDC6, CCNB1IP1, CCND1, CCND2, CCND3,
CCNE1, CD274, CD74, CD79A, CD79B, CDC73, CDH1, CDK12, CDK4, CDK6, CDK8,
CDKN1A, CDKN1B, CDKN2A, CDKN2B, CDKN2C, CDX2, CEBPA, CEP89, CHCHD7,
CHD2, CHD4, CHEK1, CHEK2, CHIC2, CHN1, CIC, CIITA, CLIP1, CLP1, CLTC, CLTCL1,
CNBP, CNOT3, CNTRL, COL1A1, COL2A1, COX6C, CREB1, CREB3L1, CREB3L2, CREBBP,
CRKL, CRLF2, CRTC1, CRTC3, CSF1R, CSF3R, CTCF, CTNNA1, CTNNB1, CUL3, CUX1,
CYLD, DAXX, DCTN1, DDB2, DDIT3, DDR2, DDX10, DDX5, DDX6, DEK, DICER1, DNM2,
DNMT3A, DOT1L, EBF1, ECT2L, EGFR, EIF3E, EIF4A2, ELF4, ELK4, ELL, ELN, EML4,
EP300, EPHA3, EPHA5, EPHA7, EPHB1, EPS15, ERBB2, ERBB3, ERBB4, ERC1, ERCC2,
ERCC3, ERCC4, ERCC5, ERG, ERRFI1, ESR1, ETV1, ETV4, ETV5, ETV6, EWSR1, EXT1,
EXT2, EZH2, EZR, FAM46C, FANCA, FANCC, FANCD2, FANCE, FANCF, FANCG, FANCL,
FAS, FAT1, FBXO11, FBXW7, FCGR2B, FCRL4, FEV, FGFR1, FGFR1OP, FGFR2, FGFR3,
FGFR4, FGF3, FGF4, FGF6, FGF10, FGF14, FGF19, FGF23, FH, FHIT, FIP1L1, FLCN,
FLI1, FLT1, FLT3, FLT4, FNBP1, FOXA1, FOXL2, FOXO1, FOXO3, FOX04, FOXP1, FRS2,
FSTL3, FUBP1, FUS, GABRA6, GAS7, GATA1, GATA2, GATA3, GATA4, GATA6, GID4, GLI1,
GMPS, GNA11, GNA13, GNAQ, GNAS, GOLGA5, GOPC, GPC3, GPHN, GPR124, GRIN2A,
GRM3, GSK3B, H3F3A, H3F3B, HERPUD1, HEY1, HGF, HIP1, HIST1H41, HIST1H41,
HLA-A, HLF, HMGA1, HMGA2, HNF1A, HNRNPA2B1, HOOK3, HOXA11, HOXA13, HOXA9,
HOXC11, HOXD13, HRAS, HSD3B1, HSP90AA1, HSP90AB1, IDH1, IDH2, IKZF1, IGF1R,
IGF2, IKBKE, IKZF1, IL2, IL21R, IL6ST, IL7R, INHBA, INPP4B, IRF2, IRF4, IRS2, ITK,
JAK1, JAK2, JAK3, JAZF1, JUN, KAT6A, KAT6B, KCNJ5, KDM5A, KDM5C, KDM6A, KDR,
KDSR, KEAP1, KEL, KIAA1549, KIAA1598, KIF5B, KIT, KLF4, KLF6, KLHL6, KLK2,
KMT2A, KMT2C, KMT2D, KRAS, KTN1, LASP1, LCK, LCP1, LHFP, LIFR, LMNA, LMO1,
LMO2, LPP, LRIG3, LRP1B, LSM14A, LYL1, LYN, LZTR1, MAF, MAFB, MAGI2, MALT1,
MAML2, MAP2K1, MAP2K2, MAP2K4, MAP3K1, MAX, MCL1, MDM2, MDM4, MECOM,
MED12, MEF2B, MEN1, MET, MITF, MKLI, MLF1, MLH1, MLLT1, MLLT10, MLLT11,
MLLT3, MLLT4, MLLT6, MN1, MNX1, MLP, MPL, MSH2, MSH6, MSI2, MSN, MTCP1,
MTOR, MUC1, MUTYH, MYB, MYC, MYCL, MYCN, MYD88, MYH11, MYH9, MYO5A, NAB2,
NACA, MBN, MCKIPSD, NCOA1, NCOA2, NCOA4, NDRG1, NF1, NF2, NFATC2, NFE2L2,
NFIB, NFKBIA, NFKB2, NIN, NKX2-1, NONO, NOTCH1, NOTCH2, NOTCH3, NPM1, NR4A3,
NRAS, NRG1, NSD1, NT5C2, NTRK1, NTRK2, NTRK3, NUMA1, NUP214, NUP93, NUP98,
NUTM1, NUTM2A, NUTM2B, OLIG2, OMD, P2RY8, PAFAH1B2, PAK3, PALB2, PARK2,
PATZ1, PAX3, PAX5, PAX7, PAX8, PBRM1, PBX1, PCM1, PCSK7, PDCD1LG2, PDE4DIP,
PDGFB, PDGFRA, PDGFRB, PER1, PHF6, PHOX2B, PICALM, PIK3CA, PIK3CB, PIK3CG,
PIK3R1, PIK3R2, PIM1, PLAG1, PLCG1, PLCG2, PML, PMS1, PMS2, POLD1, POLE, POT1,
POU2AF1, POU5F1, PPARG, PPFIBP1, PPP2R1A, PRCC, PRDM1, PRDM16, PREX2, PRF1,
PRKAR1A, PRKCI, PRKDC, PRRX1, PRSS8, PSIP1, PTCH1, PTEN, PTPN11, PTPRB,
PTPRC, PTPRK, PWWP2A, QK1, RABEP1, RAC1, RAD21, RAD50, RAD51, RAD51B, RAF1,
RALGDS, RANBP17, RANBP2, RAP1GDS1, RARA, RB1, RBM10, RBM15, RECQL4, REL,
RET, RHEB, RHOA, RHOH, RICTOR, RIT1, RMI2, RNF213, RNF43, ROS1, RPL10, RPL22,
RPL5, RPN1, RPTOR, RSPO2, RSPO3, RUNX1, RUNX1T1, SBDS, SDC4, SDHA, SDHAF2,
SDHB, SDHC, SDHD, SEPT5, SEPT6, SEPT9, SET, SETBP1, SETD2, SF3B1, SFP1, SH2B3,
SH3GL1, SLC34A2, SLC45A3, SLIT2, SMAD2, SMAD3, SMAD4, SMARCA4, SMARCB1,
SMARCE1, SMO, SOCS1, SOX2, SOX9, SOX10, SPECC1, SPEN, SPOP, SPTA1, SRC,
SRGAP3, SRSF2, SRSF3, SS18, SS18L1, SSX1, SSX2, SSX2B, SSX4, SSX4B, STAG2, STAT3,
STAT4, STAT5B, STAT6, STIL, STK11, SUFU, SUZ12, SYK, TAF1, TAF15, TAL1, TAL2,
TBL1XR1, TBX3, TCEA1, TCF12, TCF3, TCF7L2, TCL1A, TERC, TERT, TERT (promoter
only), TET1, TET2, TFE3, TFEB, TFG, TFPT, TFRC, TGFBR2, THRAP3, TLX1, TLX3,
TMPRSS2, TNFAIP3, TNFRSF14, TNFRSF17, TOP1, TOP2A, TP53, TPM3, TPM4, TPR,
TRAF7, TRIM24, TRIM27, TRIM33, TRIP11, TRRAP, TSC1, TSC2, TSHR, TTL, U2AF1, UBR5,
USP6, VEGFA, VHL, VTI1A, WAS, WHSC1, WHSC1L1, WIF1, WISP3, WRN, WT1, WWTR1,
XPA, XPC, XPO1, YWHAE, ZBTB2, ZBTB16, ZCCHC8, ZMYM2, ZNF217, ZNF331, ZNF384,
ZNF703, ZNR521, ZRSR2

The cancer marker can comprise a particular kind of mutation in a particular gene, such as a single nucleotide variant (SNV; e.g. an SNV in at least one gene of Table 2), a copy number variant (CNV; e.g. a CNV in at least one gene of Table 3), a gene fusion (e.g. involving at least one gene of Table 4), an insertion and/or deletion (Indel; e.g. an Indel in at least one gene of Table 5), or a rearrangement (e.g. a rearrangement in at least one gene of Table 6).

TABLE 2
Single nucleotide variant cancer markers

AKT1, ALK, APC, AR, ARAF, ARID1A, ATM, BRAF, BRCAL,
BRCA2, CCNDl, CCND2, CCNE1, CDHl, CDK4, CDK6, CDKN2A,
CDKN2B, CTNNB1, EGFR, ERBB2, ESR1, EZH2, FBXW7, FGFR1,
FGFR2, FGFR3, GATA3, GNA11, GNAQ, GNAS, HNF1A, HRAS,
IDH1, IDH2, JAK2, JAK3, KIT, KRAS, MAP2K1, MAP2K2,
MET, MLH1, MPL, MYC, NF1, NFE2L2, NOTCH1, NPM1, NRAS,
NTRK1, PDGFRA, PIK3CA, PTEN, PTPN11, RAF1, RB1, RET,
RHEB, RHOA, RIT1, ROS1, SMAD4, SMO, SRC, STK11, TERT,
TP53, TSC1, VHL

TABLE 3
Copy number variant cancer markers

AR, BRAF, CCND1, CCND2, CCNE1, CDK4, CDK6, EGFR, ERBB2,
FGFR1, FGFR2, KIT, KRAS, MET, MYC, PDGFRA, PIK3CA, RAF1

TABLE 4
Gene fusion cancer markers

ALK, FGFR2, FGFR3, NTRK1, RET, ROS1

TABLE 5
Gene insertion or deletion cancer markers

EGFR (exons 19 and/or 20), ERBB2 (exons 19 and/or 20), MET (exon 14
skipping)

TABLE 6
Gene rearrangement cancer markers

ALK, BRAF, BRD4, ETV4, ETV6, KIT, MYC, NTRK2, RARA,
TMPRSS2, BCL2, BRCA1, EGFR, ETV5, FGFR2, MSH2, NOTCH2,
PDGFRA, RET, BCR, BRCA2, ETV1, ETV6, FGFR3, MYB, NTRK1,
RAF1, ROS1

Disclosed herein are methods, kits and systems for detecting, quantifying, and/or analyzing at least one marker of breast cancer. Markers of breast cancer may comprise a polynucleotide or peptide corresponding to a gene selected from, but not limited to, TFF3, FAS, XBP1, IFI6, GS (glutamine synthetase), DSP, PIP5K2A, PHGDH, APOCI, NDUFA1, CD74, IGFBP7, CLAML5, and IBC-1, and combinations thereof.

Disclosed herein are methods, kits and systems for detecting, quantifying, and/or analyzing at least one marker of multiple sclerosis. Markers of multiple sclerosis may comprise a polynucleotide or peptide corresponding to a gene selected from, but not limited to, MBP, MOG, PLP, CRP, ORM, C3 proactivator, VCAM-1, ICAM-1, MMP-9, CXCL16, TNF-alpha, PCT, OPG, UGTA8, MOG, ENPP6, MOBP, CLDN11, GSN, EVI2A, BCAS1, TSPAN2, EDGE, PLP1, GJA12, GAL3ST1, ERBB3, TMEM10, PLA2G4A, ELOVL7, SGK2, MBP, FA2H, GM98, MAG, IL23A, SRPK3, PLXNB3, PRKCQ, TRF, PLEKHH1, MAL, GPR62, CPM, and ADAMTS4, and combinations thereof. Non-polynucleotide or non-peptide markers of multiple sclerosis may be selected from glycolipids, sphingomyelin, neopterin and nitric oxide metabolites. The sample may be a cerebrospinal fluid sample and the at least one marker may comprise an immunoglobulin or fragment thereof. The immunoglobulin or fragment thereof may be detected as an oligoclonal band.

Disclosed herein are methods, kits and systems for detecting, quantifying, and/or analyzing at least one marker of a hepatitis. The hepatitis may be an alcohol-induced hepatitis, a non-alcoholic steatohepatitis (NASH), a viral hepatitis, or a combination thereof. Further disclosed herein are methods, kits and systems for differentiating between NAFLD and NASH. In some instances, markers of hepatitis are selected from high levels of albumin, ER stress pathway signaling proteins, cytokines, and chemokines in circulating fluids of the human subject. In some cases high levels of these markers are observed with the onset of NASH, but before liver cell death occurs. Thus, the methods, kits and systems disclosed herein provide for interventions before NASH progresses or advances past initial stages of the disease. Some markers of hepatitis, e.g., NASH, are polynucleotides or peptides corresponding to genes selected from, but not limited to, LXR-alpha, PPAR-gamma, SREBP-1c, SREBP-2, FAS, iNOS, COX2, OPN, TFN-alpha, SOCS3, IL6, and PNPLA3 I148M.

Disclosed herein are methods, kits and systems for detecting, quantifying, and/or analyzing at least one marker of cardiovascular disease. Markers of cardiovascular disease may comprise a polynucleotide or peptide corresponding to a gene selected from, but not limited to, A2M, ACE, ADIPOQ, AGT, ALB, ALDOC, APOA1, APOA2, APOA4, APOB, APOC1, APOC2, APOC3, APOD, APOE, APOH, APOL1, BDH1, C3, C4A, C4B, CCL2, CD40LG, CETP, CHGA, CHIT1, CKB, CKM, CLU, CP, CPB2, CRP, CSF1, CTSB, CXCL8, EDN1, ENG, ENO2, ENO3, EPO, coagulation factors (F10, F11, F12, F13A1, F13B, F2, F3, F5, F7, F8, F9), FABP3, FAS, FGA, FGB, FGF2, FGG, FN1, FST, FTH1, FTL, GFAP, GGT1, GH1, GOT2, HABP2, HEXA, HGF, HP, HPX, ICAM1, IGF1, IGFBP1, IL10, IL18, IL1B, IL1RL1, IL1RN, IL2, IL6, IL6R, IL6ST, INHBA, INS, ITGA2B, LCN2, LEP, LEPR, LPA, LPL, LRP1, MAPT, myoglobin, MBP, WE, MMP1, MMP2, MMP3, MMP9, MPO, MYH11, MYH6, MYH7, MYL2, MYL3, NGF, NPPA, NPPB, ORM1, PAPPA, PDGFA, PDGFB, PF4, PGAM1, PGF, PLA2G1B, PLA2G7, PLAT, PLG, PON1, PON2, PON3, PROC, PROCR, PROS1, PROZ, PRTN3, PYGB, REN, RETN, S100B, SAA1, SAA2, SELE, SELL, SELP, SELPLG, SERPINA1, SERPINA3, SERPINA5, SERPINC1, SERPIND1, SERPINE1, SERPINF2, SERPING1, SHBG, TFPI, TGFB1, THBD, THBS1, TIMP1, TIP2, TNF, TNFRSF11B, TNFRSF1A, TNFRSF1B, TNNI3, TNNT2, TPM1, VEGFA, VTN, and VWF, and combinations thereof.

Further examples of markers are provided elsewhere herein, such as in association with diseases and conditions described herein, and the various aspects of the present disclosure.

### Panels

As discussed in the foregoing and following description, methods, systems and kits are provided herein to non-invasively detect a tissue or organ under duress as well as determine which disease or condition is affecting the tissue or organ under duress. Some kits, systems and methods disclosed herein provide for detecting or quantifying a plurality of markers and/or a plurality of tissue-specific polynucleotides that correspond to two or more genes disclosed herein. The plurality of tissue-specific polynucleotides or markers may be referred to as a panel herein. In some instances, a panel is necessary to draw an inference or conclusion about the subject's health status, condition, or status of a tissue or organ. For example, due to natural variation in genetic expression across a population or populations of subjects, determining quantities of one tissue-specific polynucleotide or a single marker may not be sufficient to determine if a tissue of interest or if any tissue is under duress. Instead, it may be necessary to determine that a majority of tissue-specific polynucleotides and/or markers are above or below threshold levels. Threshold levels may be levels of tissue-specific polynucleotides and/or markers in control subjects, e.g., subject without a disease or condition or subject with a disease or condition of interest. In some instances a control subject is a subject with disease, condition or damage of a tissue of interest. For example, a control subject with a liver condition may have cell-free liver-specific polynucleotides circulating in their blood at least at a level that is designated as a threshold level. In some cases, it may be necessary to determine that at least 50% of tissue-specific polynucleotides and/or markers of the panel are at or above threshold levels. In some cases, it may be necessary to determine that at least 55% of tissue-specific polynucleotides and/or markers of the panel are at or above threshold levels. In some cases, it may be necessary to determine that at least 60% of tissue-specific polynucleotides and/or markers of the panel are at or above threshold levels. In some cases, it may be necessary to determine that at least 65% of tissue-specific polynucleotides and/or markers of the panel are at or above threshold levels. In some cases, it may be necessary to determine that at least 70% of tissue-specific polynucleotides and/or markers of the panel are at or above threshold levels. In some cases, it may be necessary to determine that at least 75% of tissue-specific polynucleotides and/or markers of the panel are at or above threshold levels. In some cases, it may be necessary to determine that at least 80% of tissue-specific polynucleotides and/or markers of the panel are at or above threshold levels. In some cases, it may be necessary to determine that at least 85% of tissue-specific polynucleotides and/or markers of the panel are at or above threshold levels. In some cases, it may be necessary to determine that at least 90% of tissue-specific polynucleotides and/or markers of the panel are at or above threshold levels.

The methods may comprise comparing expression of a panel of markers and/or tissue-specific polynucleotides disclosed herein in a test sample from a test subject to that of a control sample from a control subject. The control subject may be a healthy subject. The control subject may be a subject with a disease or condition related to the panel markers/tissue-specific polynucleotides. If the test subject gene expression or marker levels are sufficiently similar or sufficiently different from the control subject, a conclusion or inference is made about the health state or condition of the test subject. Sufficiently similar may mean that at least 50% of the transcripts or markers in the test sample are present in a quantity that is within 10% of the quantity of the transcripts or markers in the control sample. Sufficiently similar may mean that at least 50% of the transcripts or markers in the test sample are present in a quantity that is within 25% of the quantity of the transcripts or markers in the control sample. The quantities may be absolute or relative. Sufficiently different may mean that less than 50% of the transcripts or markers in the test sample are present in a quantity that is within 10% of the quantity of the transcripts or markers in the control sample. Sufficiently similar may mean that less than 50% of the transcripts or markers in the test sample are present in a quantity that is within 25% of the quantity of the transcripts or markers in the control sample. The quantities may be absolute or relative.

Some panels disclosed herein comprise between two and a hundred tissue-specific polynucleotides and/or markers. In some instances, a panel comprises between five and a hundred tissue-specific polynucleotides and/or markers. In some instances, a panel comprises between ten and a hundred tissue-specific polynucleotides and/or markers. In some instances, a panel comprises between 10 and 150 tissue-specific polynucleotides and/or markers. In some instances, a panel comprises between 10 and a 200 tissue-specific polynucleotides and/or markers. In some instances, a panel comprises between 5 and 150 tissue-specific polynucleotides and/or markers.

### Samples

As discussed in the foregoing and following description, methods, systems and kits are provided herein to non-invasively detect a tissue or organ under duress as well as determine which disease or condition is affecting the tissue or organ under duress. Methods, kits and systems disclosed herein provide for detecting, quantifying, and/or analyzing markers and/or tissue-specific polynucleotides in a sample of a subject. The methods disclosed herein may further comprise obtaining the sample from the subject. Obtaining the sample may comprise collecting a fluid sample, such as blood, urine, cerebrospinal fluid, lymphatic fluid, bone marrow, saliva, sputum, semen, transvaginal fluid, sweat, breast fluid, and combinations thereof. Obtaining the sample may comprise performing a biopsy or using a swab to collect at least one cell or a fluid from the subject. Obtaining the sample may comprise performing a needle biopsy or a spinal tap. The methods may comprise further processing the sample. By way of non-limiting example, further processing the sample may comprise fractioning a blood sample to obtain serum or plasma.

Methods, kits and systems may be used to detect, quantify, and/or analyze at least one marker and/or tissue-specific polynucleotide in at least one sample. The methods, kits and systems may be used to detect, quantify, and/or analyze at least one marker and/or tissue-specific polynucleotide in a single sample from the subject. The methods, kits and systems may be used to detect, quantify, and/or analyze at least one marker in a first sample and at least one tissue-specific polynucleotide in a second sample, wherein the first sample and the second sample are different. The first sample and the second sample may be different based on the time of obtaining the first and second sample (e.g., sequential sampling). The first sample and the second sample may be different based on the source of the sample (e.g., cerebrospinal fluid and blood). In some embodiments, the first and second samples are different aliquots of a common parent sample.

Often, methods, kits and systems may be used to detect, quantify, and/or analyze a first marker and a first tissue-specific polynucleotide in a first sample from the subject, and a second marker and a second tissue-specific polynucleotide in a second sample from the subject. The first marker and the second marker may be the same. The first marker and the second marker may be different. The first tissue-specific polynucleotide and the second tissue-specific polynucleotide may be the same. The first tissue-specific polynucleotide and the second tissue-specific polynucleotide may be different. Although it may be advantageous to obtain a minimal number of samples, and detect, quantify and/or analyze a minimal number of markers and tissue-specific polynucleotides, the methods, kits and systems disclosed herein provide for various combinations of samples that may be required to accurately determine the presence, stage, state or risk of a disease or condition.

Some methods, kits and systems may be used to detect, quantify, and/or analyze at least one marker and/or tissue-specific polynucleotide in at least one sample from a subject and compare the presence or levels of the marker and/or tissue-specific polynucleotide in at least one sample obtained from the subject at an earlier time point. Thus, the methods, kits and systems may be useful to monitor the progression, stage, or prognosis of a disease or the effects (e.g., efficacy, toxicity) of a therapeutic treatment.

### Diseases and Conditions

As discussed in the foregoing and following description, methods, systems and kits are provided herein to non-invasively detect a tissue or organ under duress as well as determine which disease or condition is affecting the tissue or organ under duress. Methods, kits and systems disclosed herein provide for detecting, quantifying, and/or analyzing at least one marker of a disease or condition. A disease or condition may cause damage or injury to a tissue or organ of a subject. By way of non-limiting example, growth of a tumor or metastasis of a tumor into a tissue or organ may result in damage or injury to the tissue or organ. This may result in cell death, cell lysis, or cell membrane disruption, resulting in the release of nucleic acids from respective cells and the presence of cell-free, tissue-specific polynucleotides in biological fluids (e.g., blood, plasma, serum, or cerebrospinal fluid) of the subject. Any of a variety of diseases or conditions may be assessed using methods of the disclosure, either alone or in combination. Non-limiting examples of diseases or conditions include a cardiovascular disease or condition, a kidney-associated disease or condition, a prenatal or pregnancy-related disease or condition, a neurological or neuropsychiatric disease or condition, an autoimmune or immune-related disease or condition, a cancer, an infectious disease or condition, a mitochondrial disorder, a respiratory disease or condition, a gastrointestinal tract disease or condition, a reproductive disease or condition, an ophthalmic disease or condition, a musculo-skeletal disease or condition, a liver-associated disease or condition, a metabolic condition, a neurodegenerative disease or condition, or a dermal disease or condition. Conditions include non-disease conditions of a subject. For example, conditions of a subject include likelihood of response to a mode of treatment (e.g. a pharmaceutical composition) determined prior to administration, and degree of positive or negative response to such treatment after administration.

In general, the terms, “heart disease,” “heart-associated condition,” “coronary artery disease,” and “cardiovascular disease or condition” refers to a disease or condition that affects the heart or blood vessels (e.g., arteries and veins). Examples of cardiovascular diseases include, but are not limited to myocardial infarction, coronary artery disease, percutaneous transluminal coronary angioplasty (PTCA), coronary artery bypass surgery (CABG), restenosis, peripheral arterial disease, stroke, abdominal aorta aneurysm, intracranial aneurysm, large artery atherosclerotic stroke, cardiogenic stroke, early onset myocardial infarction, heart failure, pulmonary embolism, acute coronary syndrome (ACS), angina, cardiac hypertrophy, arteriosclerosis, myocarditis, pancarditis, endocarditis, hypertension, congestive heart failure, atherosclerosis, cerebrovasculardisease, declining cardiac health, ischemic heart disease, pericarditis, cardiogenic shock, alcoholic cardiomyopathy, congenital heart disease, ischemic cardiomyopathy, hypertensive cardiomyopathy, valvular cardiomyopathy, inflammatory cardiomyopathy, cardiomyopathy secondary to a systemic metabolic disease, dilated cardiomyopathy, hypertrophic cardiomyopathy, arrhythmogenic right ventricular cardiomyopathy, restrictive cardiomyopathy, noncompaction cardiomyopathy, valvular heart disease, hypertensive heart disease, myocardial ischemic attack, unstable angina, myocardial rupture, cardiogenic shock, embolism, deep vein thrombosis, arrhythmia, diabetic cardiomyopathy, mitral regurgitation, mitral valve prolapse, peripheral vascular disease, artery disease, carotid artery disease, venous diseases, cerebrovascular disease, arterial aneurysm, left ventricular hypertrophy, hypertensive renal disease, hypertensive retinal disease, vasculitis, left main disease, arterial vascular disease, venous vascular disease, thrombosis of the microcirculation, transient ischemic attack, cerebrovascular accident, limb ischemia, aneurysm, thrombosis, and superficial venous thrombosis.

In general, the term “kidney-associated disease or condition” refers to a disease or condition that affects the kidney or renal system. Examples of kidney-associated disease include, but are not limited to, chronic kidney diseases, primary kidney diseases, non-diabetic kidney diseases, glomerulonephritis, interstitial nephritis, diabetic kidney diseases, diabetic nephropathy, glomerulosclerosis, rapid progressive glomerulonephritis, renal fibrosis, Alport syndrome, IDDM nephritis, mesangial proliferative glomerulonephritis, membrano proliferative glomerulonephritis, crescentic glomerulonephritis, renal insterstitial fibrosis, focal segmental glomerulosclerosis, membranous nephropathy, minimal change disease, pauci-immune rapidly progressive glomerulonephritis, IgA nephropathy, polycystic kidney disease, Dent's disease, nephrocytinosis, Heymann nephritis, autosomal dominant (adult) polycystic kidney disease, autosomal recessive (childhood) polycystic kidney disease, acute kidney injury, nephrotic syndrome, renal ischemia, podocyte diseases or disorders, proteinuria, glomerular diseases, membranous glomerulonephritis, focal segmental glomerulonephritis, pre-eclampsia, eclampsia, kidney lesions, collagen vascular diseases, benign orthostatic (postural) proteinuria, IgM nephropathy, membranous nephropathy, sarcoidosis, kidney damage due to drugs, Fabry's disease, aminoaciduria, Fanconi syndrome, hypertensive nephrosclerosis, interstitial nephritis, Sickle cell disease, hemoglobinuria, myoglobinuria, Wegener's Granulomatosis, Glycogen Storage Disease Type 1, chronic kidney disease, chronic renal failure, low Glomerular Filtration Rate (GFR), nephroangiosclerosis, lupus nephritis, ANCA-positive pauci-immune crescentic glomerulonephritis, chronic allograft nephropathy, nephrotoxicity, renal toxicity, kidney necrosis, kidney damage, glomerular and tubular injury, kidney dysfunction, nephritic syndrome, acute renal failure, chronic renal failure, proximal tubal dysfunction, acute kidney transplant rejection, chronic kidney transplant rejection, non IgA mesangioproliferative glomerulonephritis, postinfectious glomerulonephritis, vasculitides with renal involvement of any kind, any hereditary renal disease, any interstitial nephritis, renal transplant failure, kidney cancer, kidney disease associated with other conditions (e.g., hypertension, diabetes, and autoimmune disease), a primary kidney disease, a collapsing glomerulopathy, a dense deposit disease, a cryoglobulinemia-associated glomerulonephritis, an Henoch-Schónlein disease, a postinfectious glomerulonephritis, a microscopic polyangitis, a Churg-Strauss syndrome, an anti-GBM-antibody mediated glomerulonephritis, amyloidosis, a monoclonal immunoglobulin deposition disease, a fibrillary glomerulonephritis, an immunotactoid glomerulopathy, ischemic tubular injury, a medication-induced tubulo-interstitial nephritis, a toxic tubulo-interstitial nephritis, an infectious tubulo-interstitial nephritis, a bacterial pyelonephritis, a viral infectious tubulo-interstitial nephritis which results from a polyomavirus infection or an HIV infection, a metabolic-induced tubulo-interstitial disease, a mixed connective disease, a cast nephropathy, a crystal nephropathy which may results from urate or oxalate or drug-induced crystal deposition, an acute cellular tubulo-interstitial allograft rejection, an obstructive disease of the kidney, an atheroembolic renal disease, a mixed connective tissue disease, a polyarteritis nodosa, an acute cellular vascular allograft rejection, an acute humoral allograft rejection, early renal function decline (ERFD), end stage renal disease (ESRD), renal vein thrombosis, acute tubular necrosis, acute interstitial nephritis, established chronic kidney disease, renal artery stenosis, ischemic nephropathy, uremia, drug and toxin-induced chronic tubulointerstitial nephritis, reflux nephropathy, kidney stones, Goodpasture's syndrome, and hydronephrosis.

In general, the term “liver-associated disease or condition” refers to a disease or condition that affects the liver. The liver-associated disease or condition may be selected from steatosis (fatty liver), steatohepatitis, hepatitis, cirrhosis, liver cancer, and fibrosis. Cirrhosis may be due to alcohol consumption or hepatitis. Hepatitis may be alcoholic hepatitis, autoimmune hepatitis or viral hepatitis. Liver cancers may include hepatocellular carcinoma, cholangiocarcinoma, angiosarcoma and hemangiosarcoma. The liver-associated disease or condition may be caused by a parasitic infection (e.g. fascioliasis). The liver-associated disease or condition may be a hereditary/genetic disease (e.g. hemochromatosis, Wilson's disease, Gilbert's syndrome). The liver-associated disease or condition may be at least partially due to alcohol consumption. The liver-associated disease or condition may not be due to alcohol consumption.

The liver-associated disease or condition may be non-alcoholic fatty liver disease (NAFLD) or non-alcoholic steatohepatitis (NASH). Generally, NAFLD is characterized by accumulation of lipids in the liver, with little to no inflammation or fibrosis. NASH is a more severe disease. In addition to accumulation of lipids in the liver, NASH is characterized by inflammation, necrosis fibrosis, cirrhosis, or a combination thereof. NAFLD may develop before NASH. Gene expression changes or chromosomal modifications may be indicative of a transition from NAFLD to NASH. The liver-associated disease or condition may be associated with obesity. The liver-associated disease or condition may be associated with a body mass index (BMI) that is indicative of being overweight or obese. Generally, a subject having a BMI greater than 25 is considered overweight and a subject having a BMI greater than 30 is considered obese. For instance a subject having the liver-associated disease or condition may have a BMI greater than 25. The subject having the liver-associated disease or condition may have a BMI greater than 26. The subject having the liver-associated disease or condition may have a BMI greater than 30. The liver-associated condition may be liver failure.

In general, the term “metabolic condition” refers to a disease or condition selected from obesity, insulin resistance, decreased insulin sensitivity, and combinations thereof. Provided herein are methods utilizing subcutaneous fat-specific transcripts and visceral fat-specific transcripts to monitor progress of a metabolic condition and response to medications for metabolic conditions. Deposition of visceral fat is commonly associated with progression of diabetes and weight gain, while loss of cutaneous fat is associated with aging, use of steroids, substantial weight loss or other wasting conditions. Deposition of visceral fat in particular, has been associated with increased cardiac risk, whereas its loss is associated with risk improvement. Increased activity and turnover (as opposed to necrosis) of adipose cells may be associated with increased plasma levels of fat-specific transcripts or an improvement in the visceral fat-specific to subcutaneous fat-specific ratio of transcripts in blood. For example, caloric restriction or a change to a low carbohydrate diet and replacement with dietary fats (e.g., Atkins diet) has been shown to greatly decrease the deposition of visceral fat. Dietary intervention, in addition to blood pressure lowering drugs (e.g. ACE inhibitors and ARBs), and serum glucose lowering drugs such as metformin, GLP-1 agonists, DPP-4 inhibitors, and SGLT2 inhibitors, may improve the fat ratio while decreasing end-organ damage in kidneys, retina, liver, heart, artery and pancreas. In contrast, hypoglycemic agents such as insulin whose predominant mechanism is gluconeogenic and anabolic, may be expected to worsen visceral to cutaneous fat ratios, and exacerbate damage to the liver, and other organs by increasing local fat synthesis and storage.

In some embodiments, the methods disclosed herein comprise detecting and/or analyzing at least one polynucleotide (e.g., RNA, DNA) associated with a liver-associated disease or condition. The at least one polynucleotide may comprise a gene or genetic transcript or portions thereof associated with a liver-associated disease or condition. The portion of the gene or genetic transcript thereof may comprise a sufficient number of nucleotides to determine the portion of the gene or genetic transcript thereof is associated with a gene of interest, mutants thereof, chemical modifications thereof, and splice variants thereof. The polynucleotide may encode a protein or identifiable portion thereof. The gene of interest, by way of non-limiting example, may be selected from a gene associated with a cellular function selected from lipid metabolism, lipid storage, lipid transport (uptake/efflux), cholesterol metabolism, cholesterol storage, cholesterol transport (cellular uptake/efflux), inflammation, extracellular matrix formation, drug metabolism, drug transport (cellular uptake/efflux), vitamin storage, vitamin uptake, vitamin metabolism, and apoptosis.

In general, the term “prenatal or pregnancy-related disease or condition” refers to a disease or condition affecting a pregnant woman, embryo, or fetus. Prenatal or pregnancy-related conditions can also refer to any disease, disorder, or condition that is associated with or arises, either directly or indirectly, as a result of pregnancy. These diseases or conditions can include any and all birth defects, congenital conditions, or hereditary diseases or conditions. Examples of prenatal or pregnancy-related diseases include, but are not limited to, Rhesus disease, hemolytic disease of the newborn, beta-thalassemia, sex determination, determination of pregnancy, a hereditary Mendelian genetic disorder, chromosomal aberrations, a fetal chromosomal aneuploidy, fetal chromosomal trisomy, fetal chromosomal monosomy, trisomy 8, trisomy 13 (Patau Syndrome), trisomy 16, trisomy 18 (Edwards syndrome), trisomy 21 (Down syndrome), X-chromosome linked disorders, trisomy X (XXX syndrome), monosomy X (Turner syndrome), XXY syndrome, XYY syndrome, XXXY syndrome, XXYY syndrome, XYYY syndrome, syndrome, XXXYY syndrome, XXYYY syndrome, Fragile X Syndrome, fetal growth restriction, cystic fibrosis, a hemoglobinopathy, fetal death, fetal alcohol syndrome, sickle cell anemia, hemophilia, Klinefelter syndrome, dup(17)(p11.2p1.2) syndrome, endometriosis, Pelizaeus-Merzbacher disease, dup(22)(q11.2q11.2) syndrome, cat eye syndrome, cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Prader-Willi syndrome, Kallmann syndrome, microphthalmia with linear skin defects, adrenal hypoplasia, glycerol kinase deficiency, Pelizaeus-Merzbacher disease, testis-determining factor on Y, azospermia (factor a), azospermia (factor b), azospermia (factor c), 1p36 deletion, phenylketonuria, Tay-Sachs disease, adrenal hyperplasia, Fanconi anemia, spinal muscular atrophy, Duchenne's muscular dystrophy, Huntington's disease, myotonic dystrophy, Robertsonian translocation, Angelman syndrome, tuberous sclerosis, ataxia telangietasia, open spina bifida, neural tube defects, ventral wall defects, small-for-gestational-age, congenital cytomegalovirus, achondroplasia, Marfan's syndrome, congenital hypothyroidism, congenital toxoplasmosis, biotimidase deficiency, galactosemia, maple syrup urine disease, homocystinuria, medium-chain acyl Co-A dehydrogenase deficiency, structural birth defects, heart defects, abnormal limbs, club foot, anencephaly, arhinencephaly/holoprosencephaly, hydrocephaly, anophthalmos/microphthalmos, anotia/microtia, transposition of great vessels, tetralogy of Fallot, hypoplastic left heart syndrome, coarctation of aorta, cleft palate without cleft lip, cleft lip with or without cleft palate, esophageal atresia/stenosis with or without fistula, small intestine atresia/stenosis, anorectal atresia/stenosis, hypospadias, indeterminate sex, renal agenesis, cystic kidney, preaxial polydactyly, limb reduction defects, diaphragmatic hernia, blindness, cataracts, visual problems, hearing loss, deafness, X-linked adrenoleukodystrophy, Rett syndrome, lysosomal disorders, cerebral palsy, autism, aglossia, albinism, ocular albinism, oculocutaneous albinism, gestational diabetes, Arnold-Chiari malformation, CHARGE syndrome, congenital diaphragmatic hernia, brachydactlia, aniridia, cleft foot and hand, heterochromia, Dwarnian ear, Ehlers Danlos syndrome, epidermolysis bullosa, Gorham's disease, Hashimoto's syndrome, hydrops fetalis, hypotonia, Klippel-Feil syndrome, muscular dystrophy, osteogenesis imperfecta, progeria, Smith Lemli Opitz syndrome, chromatopsia, X-linked lymphoproliferative disease, omphalocele, gastroschisis, pre-eclampsia, eclampsia, pre-term labor, premature birth, miscarriage, delayed intrauterine growth, ectopic pregnancy, hyperemesis gravidarum, morning sickness, or likelihood for successful induction of labor.

In general, the terms “neurological disease or condition,” “neuropsychiatric disease or condition,” and “neurodegenerative disease or condition,” refer to a disease or condition that affects the nervous system. Examples of neurological, neurodegenerative and neuropsychiatric diseases or conditions include, but are not limited to, head trauma, stroke, ischemic stroke, hemorrhagic stroke, subarachnoid hemorrhage, intra cranial hemorrhage, transient ischemic attack, vascular dementia, corticobasal ganglionic degeneration, encephalitis, epilepsy, Landau-Kleffner syndrome, hydrocephalus, pseudotumor cerebri, thalamic diseases, meningitis, myelitis, movement disorders, essential tremor, spinal cord diseases, syringomyelia, Alzheimer's disease (early onset), Alzheimer's disease (late onset), multi-infarct dementia, Pick's disease, Huntington's disease, Parkinson's disease, Parkinson syndromes, dementia, corticobasal degeneration, multiple system atrophy, progressive supranuclear palsy, Lewy body disease, Dandy-Walker syndrome, Friedreich ataxia, Machado-Joseph disease, migraine, schizophrenia, mood disorders and depression, dementia with lewy bodies (DLB), frontotemporal dementia (FTD), various forms of vascular dementia (VD), subcortical vascular dementia (Binswanger's disease), autism, developmental retardations, motor neuron diseases, amyotrophic lateral sclerosis (ALS), neuronal or brain damage, hypoxia of the brain, cerebral palsy (CP), memory disorders, movement disorders, corticalbasal ganglionic degeneration, forms of multiple system atrophy, stroke-related disorders, cerebrovascular accidents, post-irradiation encephalopathy with seizures, vascular Parkinsonism, thalamic cerebrovascular accidents, chronic inflammatory demyelinating polyneuropathy, alcohol related dementia, semantic dementia, ataxia, atypical Parkinsonism, dystonia, progressive supranuclear palsy, essential tremor, mild cognitive impairment, multiple sclerosis, neuropathies, congophilic amyloid angiopathy, Creutzfeldt-Jakob Disease, AIDS dementia complex, depression, anxiety disorder, phobia, Bell's Palsy, epilepsy, encephalitis, neuromuscular disorders, neurooncological disorders, brain tumors, neurovascular disorders, neuroimmunological disorders, neuro-otological disease, neurotrauma including spinal cord injury, pain including neuropathic pain, pediatric neurological and neuropsychiatric disorders, sleep disorders, Tourette syndrome, corticalbasal ganglionic degeneration, Alzheimer's disease combined with multi-infarct dementia, Alzheimer's disease combined with Lewy body dementia, Parkinson's disease combined with Lewy body dementia, Alzheimer's and Parkinson's disease combined with Lewy body dementia, frontotemporal dementia combined with chronic inflammatory demyelinating polyneuropathy, attention deficit hyperactivity disorder, obsessive-compulsive disorder, mental retardation, autistic spectrum disorders, opsoclonus-myoclonus syndrome (OMS) seizures, articulation disorder, learning disabilities (e.g., reading or arithmetic), verbal or performance aptitude deficits, attention deficit disorder, amyloid diseases, prion diseases, Tauopathies, Alpha-Synucleinopathies, and addictive states such as those caused by at least one of: cocaine, nicotine, alcohol, food, ecstasy, methcathinone, caffeine, opium, heroin, marijuana, amphetamine, methamphetamine, or gambling.

In general, the term “an autoimmune or immune-related disease or condition” refers to a disease or condition that affects the function of the immune system. Examples of autoimmune or immune-related diseases or conditions include, but are not limited to, antiphospholipid syndrome, systemic lupus erythematosus, rheumatoid arthritis, autoimmune vasculitis, celiac disease, autoimmune thyroiditis, post-transfusion immunization, maternal-fetal incompatibility, transfusion reactions, immunological deficiency such IgA deficiency, common variable immunodeficiency, drug-induced lupus, diabetes mellitus, Type I diabetes, Type II diabetes, juvenile onset diabetes, juvenile rheumatoid arthritis, psoriatic arthritis, multiple sclerosis, immunodeficiency, allergies, asthma, psoriasis, atopic dermatitis, allergic contact dermatitis, chronic skin diseases, chemotherapy-induced injury, graft-vs-host diseases, bone marrow transplant rejection, Ankylosing spondylitis, atopic eczema, Pemphigus, Behcet's disease, chronic fatigue syndrome fibromyalgia, chemotherapy-induced injury, myasthenia gravis, glomerulonephritis, allergic retinitis, systemic sclerosis, subacute cutaneous lupus erythematosus, cutaneous lupus erythematosus including chilblain lupus erythematosus, Sjogren's syndrome, autoimmune nephritis, autoimmune vasculitis, autoimmune hepatitis, autoimmune carditis, autoimmune encephalitis, autoimmune mediated hematological diseases, lc-SSc (limited cutaneous form of scleroderma), dc-SSc (diffused cutaneous form of scleroderma), autoimmune thyroiditis (AT), Grave's disease (GD), myasthenia gravis, multiple sclerosis (MS), transplant rejection, immune aging, rheumatic/autoimmune diseases, spondyloarthropathy, psoriasis, psoriatic arthritis, myositis, scleroderma, dermatomyositis, autoimmune vasculitis, mixed connective tissue disease, idiopathic thrombocytopenic purpura, Crohn's disease, human adjuvant disease, osteoarthritis, juvenile chronic arthritis, a spondyloarthropathy, an idiopathic inflammatory myopathy, systemic vasculitis, sarcoidosis, autoimmune hemolytic anemia, autoimmune thrombocytopenia, thyroiditis, immune-mediated renal disease, a demyelinating disease of the central or peripheral nervous system, idiopathic demyelinating polyneuropathy, Guillain-Barre syndrome, a chronic inflammatory demyelinating polyneuropathy, a hepatobiliary disease, infectious or autoimmune chronic active hepatitis, primary biliary cirrhosis, granulomatous hepatitis, sclerosing cholangitis, inflammatory bowel disease, gluten-sensitive enteropathy, Whipple's disease, an autoimmune or immune-mediated skin disease, a bullous skin disease, erythema multiforme, allergic rhinitis, atopic dermatitis, food hypersensitivity, urticaria, an immunologic disease of the lung, eosinophilic pneumonias, idiopathic pulmonary fibrosis, hypersensitivity pneumonitis, a transplantation associated disease, graft rejection, psoriatic arthritis, psoriasis, dermatitis, polymyositis/dermatomyositis, toxic epidermal necrolysis, systemic scleroderma and sclerosis, responses associated with inflammatory bowel disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, mysathenia gravis, antigen-antibody complex mediated diseases, anti-glomerular basement membrane disease, anti-phospholipid antibody syndrome, allergic neuritis, Bechet disease, Castleman's syndrome, Goodpasture's syndrome, Lambert-Eaton Myasthenic Syndrome, Reynaud's syndrome, Sjorgen's syndrome, Stevens-Johnson syndrome, pemphigoid bullous, pemphigus, autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), autoimmune thrombocytopenia, autoimmune disease of the testis and ovary including autoimmune orchitis and oophoritis, primary hypothyroidism, autoimmune endocrine diseases including autoimmune thyroiditis, chronic thyroiditis (Hashimoto's Thyroiditis), subacute thyroiditis, idiopathic hypothyroidism, Addison's disease, autoimmune polyglandular syndromes (or polyglandular endocrinopathy syndromes), Sheehan's syndrome, autoimmune hepatitis, lymphoid interstitial pneumonitis (HIV), bronchiolitis obliterans (non-transplant) vs NSIP, large vessel vasculitis (including polymyalgia rheumatica and giant cell (Takayasu's) arteritis), medium vessel vasculitis (including Kawasaki's disease and polyarteritis nodosa), ankylosing spondylitis, Berger's disease (IgA nephropathy), rapidly progressive glomerulonephritis, primary biliary cirrhosis, Celiac sprue (gluten enteropathy), cryoglobulinemia, and amyotrophic lateral sclerosis (ALS).

In general, the term “cancer” refers to various types of malignant neoplasms, most of which can invade surrounding tissues, and may metastasize to different sites. In general, the terms “neoplasm” and “tumor” refer to an abnormal tissue that grows by cellular proliferation more rapidly than normal and continues to grow after the stimuli that initiated proliferation is removed. Such abnormal tissue shows partial or complete lack of structural organization and functional coordination with the normal tissue which may be either benign (benign tumor) or malignant (malignant tumor). Examples of general categories of cancer include, but are not limited to, carcinomas (malignant tumors derived from epithelial cells such as, for example, common forms of breast, prostate, lung and colon cancer), sarcomas (malignant tumors derived from connective tissue or mesenchymal cells), lymphomas (malignancies derived from hematopoietic cells), leukemias (malignancies derived from hematopoietic cells), germ cell tumors (tumors derived from totipotent cells; in adults most often found in the testicle or ovary; in fetuses, babies and young children, most often found on the body midline, particularly at the tip of the tailbone), blastic tumors (a typically malignant tumor which resembles an immature or embryonic tissue) and the like. Additional examples of types of neoplasms include but are not limited to those neoplasms associated with cancers of neural tissue, blood forming tissue, breast, skin, bone, prostate, ovaries, uterus, cervix, liver, lung, brain, larynx, gallbladder, pancreas, rectum, parathyroid, thyroid, adrenal gland, immune system, head and neck, colon, stomach, bronchi, and/or kidneys.

### Numbered Embodiments

The disclosure is further understood through review of the numbered embodiments recited herein. 1. A method of detecting a cardiovascular disease (CVD) biosignature in a biological fluid from a human subject, comprising the steps of (a) measuring a marker level in the biological fluid, wherein the marker is selected from a cholesterol, a lipid, an inflammatory mediator, a lipid mediator, and a sterol mediator; and (b) quantifying a quantity of cardiovascular ribonucleic acids (RNA) in the biological fluid, wherein a threshold marker level and a threshold quantity of liver RNA indicates a CVD biosignature. 2. The method of embodiment 1, wherein the at least one marker comprises a polynucleotide or protein encoded by a gene selected from the group consisting of: TPH1, CNTN4, CASQ2, MYOCD, FHL5, ATRNL1, RPS6KA6, RYR2, NPR3, ACADL, PLCB4, ITLN1, FIBIN, SCRG1, MRAP2, CNN1, ANGPTL1, SLC22A3, PRUNE2, PLD5, NEGR1, SEMA3D, NPR1, PDZRN3, NPNT, PLN, MPP6, SBSPON, THRB, NEXN, TTLL7, PLIN2, CCR1, SELF, MMRN1, CD163, RGS1, NPL, CD180, C7, FPR3, ST8SIA2, ASB18, MYL3, PRSS42, LRRC10, TNNI3, MYL2, SMCO1, CCDC141, MYH7, RD3L, MYBPC3, TNNT2, SCN5A, GJA3, CSRP3, MT1HL1, MYOZ2, XIRP1, KLHL31, PLEKHA5, ANKRD46, PIK3R1, TPR, TRAK2, ALDH5A1, MGEA5, DUT, FAM134B, ARIH2, COL21A1, CBLB, SOBP, SLC16A7, ANP32E, PCMTD2, and EMCN. 3. The method of embodiment 1, wherein the cardiovascular disease is atheroma and the marker is a polynucleotide or protein encoded by a gene selected from the group consisting of: TPH1, CNTN4, CASQ2, MYOCD, FHL5, ATRNL1, RPS6KA6, NPR3, RYR2, ACADL, PLCB4, ITLN1, FIBIN, SCRG1, MRAP2, CNN1, ANGPTL1, SLC22A3, PRUNE2, PLDS, NEGR1, SEMA3D, NPR1, PDZRN3, NPNT, PLN, MPP6, SBSPON, THRB, NEXN, and TTLL7. 4. The method of embodiment 1, wherein the cardiovascular disease is diabetic ischemic cardiomyopathy and the marker is a polynucleotide or protein encoded by a gene selected from the group consisting of: NPR3, PLEHA5, ANKRD46, PIK3R1, TPR, TRAK2, ALDH5A1, MGEA5, DUT, FAM134B, ARIH2, PIK3R1, COL21A1, CBLB, SOBP, SLC16A7, ANP32E, and PCMTD2. 5. The method of any one of embodiments 1-4, wherein the quantity of cardiovascular RNA is substantially greater than that of at least one reference subject that does not have CVD. 6. The method of any one of embodiments 1-4, wherein the quantity of cardiovascular RNA does not differ substantially from that of at least one reference subject that has CVD. 7. The method of any one of embodiments 1-4, comprising comparing the quantity of the cardiovascular RNA to an average cardiovascular RNA level in a plurality of subjects suffering from CVD. 8. The method of embodiment 7, wherein the quantity of the cardiovascular RNA being equal to or greater than the average levels indicates the human subject suffers from CVD. 9. The method of any one of embodiments 1-4, comprising detecting the CVD biosignature when the quantity of the cardiovascular RNA is at least equal to or greater than those of at least one subject with CVD. 10. The method of any one of embodiments 1-9, wherein measuring the quantity of cardiovascular RNA to the biological fluid comprises measuring the relative contribution of cardiovascular RNA to total circulating ribonucleic acids. 11. The method of any one of embodiments 1-10, wherein the cardiovascular RNA does not encode proteins implicated in CVD. 12. The method of any one of embodiments 1-10, wherein the cardiovascular RNA does not encode proteins upregulated in a liver of a reference subject with CVD. 13. The method of any one of embodiments 1-12, wherein the quantity of the cardiovascular RNA not differing significantly from corresponding reference levels indicative of a reference cardiovascular health status indicates the human subject's cardiovascular health status is similar to the reference cardiovascular health status. 14. The method of any one of embodiments 1-13, comprising obtaining a second biological fluid, and detecting a CVD biosignature in the second biological fluid. 15. The method of embodiment 14, wherein the second biological fluid is obtained subsequent to a CVD intervention. 16. The method of embodiment 14, wherein the CVD intervention comprises at least one of reducing alcohol intake, reducing caloric intake, increasing exercise, reducing cholesterol level, reducing inflammation and improving insulin sensitivity. 17. The method of embodiment 14, wherein the CVD intervention comprises consuming a compound selected from the group consisting of: a cholesterol-regulating compound, a lipid-regulating compound, an anti-inflammatory compound, and an insulin sensitizing compound. 18. The method of any one of embodiments 1-17, wherein the cardiovascular RNA is RNA that is predominantly expressed in a tissue selected from the group consisting of: heart, aorta, coronary artery, vascular smooth muscle and endothelium. 19. The method of any one of embodiments 1-18, wherein cardiovascular RNA is RNA expressed at a substantially higher level in a cardiovascular tissue than in any other tissue of the human subject. 20. The method of any one of embodiments 1-19, wherein the cardiovascular RNA is RNA that is predominantly expressed in coronary artery or aorta. 21. The method of any one of embodiments 1-20, wherein the cardiovascular RNA is RNA that is predominantly expressed in cells selected from endothelial cells, vascular smooth muscle cells, renal cells and cardiomyocytes. 22. The method of any one of embodiments 1-21, wherein the cardiovascular RNA corresponds to a gene selected from the group consisting of: ACTC1, ANKRD1, ASB18, BMP10, CASQ2, CCDC141, CHRNE, CORIN, CSRP3, DAND5, FABP3, GJA3, KLHL31, LRRC10, MT1HL1, MYBPC3, MYBPHL, MYH6, MYH7, MYL2, MYL3, MYL4, MYL7, MYOZ2, MYZAP, NPPA, NPPB, PLN, POPDC2, PPP1R1C, PRSS42, RD3L, RMB20, RYR2, SBK2, SBK3, SCN5A, SMCO1, ST8SIA2, TBX20 TECRL, TNNI3, TNNI3K, TNNT2, and XIRP1. 23. The method of embodiment 1, wherein the cardiovascular RNA is coronary artery RNA and corresponds to a gene selected from the group consisting of: CNTN4, CASQ2, MYOCD, FHL5, NPR3, ACADL, FIBIN, MRAP2, CNN1, SLC22A3, SEMA3D, NPR1, NPNT, PLN, SBSPON, C7, and FPR3. 24. The method of any one of embodiments 1-23, comprising measuring a quantity of deoxyribonucleic acids (DNA) in the biological fluid, wherein the DNA has a cardiovascular methylation pattern of at least one locus. 25. The method of embodiment 24, wherein the quantity of DNA having a cardiovascular methylation pattern is substantially higher than that of at least one reference subject that does not have CVD. 26. The method of any one of embodiments 24-25, wherein the quantity of DNA having a cardiovascular methylation pattern does not differ substantially from that of at least one reference subject that has CVD. 27. The method of any one of embodiments 24-26, wherein measuring the quantity of DNA having a cardiovascular methylation pattern of at least one locus to the biological fluid comprises measuring the relative contribution of DNA having a cardiovascular methylation pattern of at least one locus to total DNA in the biological fluid. 28. The method of any one of embodiments 24-27, wherein the at least one locus of the methylated DNA is not implicated in CVD. 29. The method of any one of embodiments 24-28, wherein the at least one locus of the methylated DNA is not differentially methylated between a healthy cardiovascular tissue and a cardiovascular tissue affected by CVD. 30. The method of any one of embodiments 24-29, comprising comparing methylation status of at least one locus of the methylated DNA to a reference, wherein methylation above a threshold indicates an overrepresentation of cardiovascular DNA in the biological fluid. 31. The method of any one of embodiments 24-30, comprising sequencing at least one DNA loci and at least one RNA in the biological fluid. 32. The method of any one of embodiments 1-31, wherein the biological fluid is plasma or serum. 33. The method of any one of embodiments 1-32, wherein the cardiovascular RNA is freely circulating RNA. 34. A method of detecting a non-alcoholic steatohepatitis (NASH) biosignature in a biological fluid from a human subject, comprising the steps of (a) measuring a marker level in the biological fluid, wherein the marker is selected from a cholesterol, a lipid, an inflammatory mediator, a lipid mediator, and a cholesterol mediator; and (b) measuring a quantity of liver ribonucleic acids (RNA) in the biological fluid, wherein a threshold marker level and a threshold quantity of liver RNA indicates a NASH biosignature. 35. The method of embodiment 34, wherein the marker comprises at least one polynucleotide or protein encoded by a gene selected from the group consisting of: LXR-alpha, PPAR-gamma, SREBP-1c, SREBP-2, FAS, iNOS, COX2, OPN, TFN-alpha, SOCS3, IL6, and PNPLA3 I148M 36. The method of embodiment 34 or 35, wherein the cholesterol mediator is selected from a polynucleotide or protein encoded by a gene selected from the group consisting of: LXR-alpha, SREBP-1c, and SREBP-2. 37. The method of any one of embodiments 34-36, wherein the inflammatory mediator is a polynucleotide or protein encoded by a gene selected from the group consisting of: iNOS, COX2, OPN, TFN-alpha, SOCS3 and IL-6. 38. The method of any of embodiments 34-37, wherein the lipid mediator is selected from a polynucleotide or protein encoded by a gene selected from the group consisting of: PPAR-gamma, FAS, and PNPLA3 I148M. 39. The method of any one of embodiments 34-38, wherein the threshold quantity of liver RNA is substantially greater than that of at least one reference subject that does not have NASH. 40. The method of any one of embodiments 34-39, wherein the threshold quantity of liver RNA does not differ substantially from that of at least one reference subject that has NASH. 41. The method of any one of embodiments 34-40, comprising comparing the quantity of liver RNA to respective reference levels, wherein the respective reference levels are average levels in a plurality of subjects suffering from NASH. 42. The method of any one of embodiments 34-41, wherein the threshold quantity of liver RNA being equal to or substantially greater than the average levels indicates the human subject suffers from NASH. 43. The method of any one of embodiments 34-42, comprising detecting the NASH biosignature when the threshold quantity of liver RNA is at least equal to or substantially greater than those of at least one subject with NASH. 44. The method of any one of embodiments 34-43, wherein measuring the quantity of liver RNA comprises measuring the relative contribution of liver RNA to a nucleic acid population selected from total RNA of the biological fluid and total nucleic acids of the biological fluid. 45. The method of any one of embodiments 34-44, wherein the liver RNA does not encode proteins implicated in NASH. 46. The method of any one of embodiments 34-45, wherein the liver RNA does not encode proteins upregulated in a liver of a reference subject with NASH. 47. The method of any one of embodiments 34-46, wherein the quantity of liver RNA not differing significantly from corresponding reference levels indicative of a reference liver health status indicates the human subject's liver health status is similar to the reference liver health status. 48. The method of any one of embodiments 34-47, comprising obtaining a second biological fluid, and detecting a NASH biosignature in the second biological fluid. 49. The method of embodiment 48, wherein the second biological fluid is obtained subsequent to a NASH intervention. 50. The method of embodiment 49, wherein the NASH intervention comprises at least one of reducing alcohol intake, reducing caloric intake, increasing exercise, undergoing gastric bypass surgery, reducing cholesterol level, reducing inflammation and improving insulin sensitivity. 51. The method of embodiment 49 or 50, wherein the NASH intervention comprises consuming a compound selected from a cholesterol-regulating compound, an anti-inflammatory compound, and an insulin sensitizing compound. 52. The method of any one of embodiments 34-51, wherein the liver RNA is RNA that is predominantly expressed in a human liver. 53. The method of any one of embodiments 34-52, wherein liver RNA is RNA expressed substantially higher in liver than in any other tissue of the human subject. 54. The method of any one of embodiments 34-53, wherein the liver RNA corresponds to a gene selected from the group consisting of: 1810014F10RIK, A1BG, ABCC2, ABCC6, ABCG5, ANG, ANGPTL3, ACOX2, ACSM2A, ADH1A, ADH1C, ADH6, AFM, AFP, AGXT, AHSG, AKR1C4, AKR1D1, ALB, ALDH1B1, ALDH4A1, ALDOB, AMBP, AOC3, APCS, APOA1, APOA2, APOA5, APOB, APOC1, APOC2, APOC3, APOC4, APOE, APOF, APOH, APOM, ARID1A, ARSE, ASL, AQP9, ASGR1, ASGR2, ATF5, C4A, C4BPA, C6, C8A, C8B, C8G, C9, CAPN5, CES1, CES2, CFHR1, CFHR2, CFHR3, CFHR4, CFHR5, CHD2, CIDEB, CPN1, CRLF1, CRYAA, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F12, DIO1, DAK, DCXR, F10, F12, F2, F9, FAH, FCN2, FETUB, FGA, FGB, FGG, FMO3, FTCD, G6PC, GPC3, GALK1, GAMT, GBA, GBP7, GCKR, GLYAT, GNMT, GPT, GSTM1, HAAO, HAMP, HAO1, HGD, HGFAC, HMGCS2, haptoglobin, HPN, HPR, HPX, HRG, HSD11B1, HSD17B6, HLF, IGF2, IL1RN, IGFALS, IQCE, ITIH1, ITIH2, ITIH4, JCLN, KHK, KLK13, LBP, LECT2, LOC55908, LPA, MASP2, MBL2, MGMT, MUPCDH, NHLH2, NNMT, NSFL1C, OATP1B1, ORM2, PCK1, PEMT, PGC, PLG, PKLR, PLGLB2, POLR2C, PON1, PON3, PROC, PXMP2, RBP4, RDH16, RET, SAA4, SARDH, SDS, SDSL, SEC14L2, SERPINA4, SERPINA7, SERPINA10, SERPINA11, SERPINC1, SERPIND1, SLCO1B1, SLC10A1, SLC22A1, SLC22A7, SLC22A10, SLC25A47, SLC27A5, SLC38A3, SLC6A12, SPP2, TAT, TBX3, TF, TIM2, TMEM176B, TST, UPB1, UROC1, VTN, WNT7A, C2, C2ORF72, CPB2, CYP4F11, CYP4F2, DUSP9, GABBR1, HP, HPD, IGSF1, IL17RB, ITIH2, ITIH3, LCAT, LGALS4, MAT1A, MST1, MSTP9, NR0B2, NR1I2, ORM1, RELN, RGN, RHBG, SAA4, SERPINA5, SERPINA7, SERPINC1, SERPINF2, SLC2A2, SULT1A2, SULT2A1, TCP10L, TNNI2, UGT2B15, and UGT2B17. 55. The method of any one of embodiments 34-54, comprising measuring a quantity of a deoxyribonucleic acid (DNA) in the biological fluid, wherein the DNA has a liver methylation pattern of at least one locus. 56. The method of embodiment 55, wherein the quantity of DNA having a liver methylation pattern is substantially greater than that of at least one reference subject that does not have NASH. 57. The method of embodiment 55 or 56, wherein the quantity of DNA having a liver methylation pattern does not differ substantially from that of at least one reference subject that has NASH. 58. The method of any one of embodiments 55-57, wherein measuring the quantity of DNA having a liver methylation pattern of at least one locus comprises measuring the relative contribution of DNA having a liver methylation pattern of at least one locus to total DNA in the biological fluid. 59. The method of any one of embodiments 55-58, wherein the at least one locus of the methylated DNA is not implicated in NASH. 60. The method of any one of embodiments 55-59, wherein the at least one locus of the methylated DNA is not differentially methylated between a healthy liver tissue and a liver affected by NASH. 61. The method of any one of embodiments 55-60, comprising comparing methylation status of at least one locus of the methylated DNA to a reference, wherein methylation above a threshold indicates an overrepresentation of liver DNA in the biological fluid. 62. The method of any one of embodiments 55-61, comprising sequencing at least one DNA loci and at least one RNA in the biological fluid. 63. The method of any one of embodiments 34-62, wherein the biological fluid is plasma or serum. 64. The method of any one of embodiments 34-63, wherein the liver RNA is freely circulating RNA. 65. A method of monitoring a human subject with a chronic metabolic condition for a presence or increased risk of at least one complication at least one tissue, comprising the steps of: (a) obtaining a biological fluid from the subject; (b) measuring a marker level in the biological fluid, wherein the marker is selected from a cholesterol, a lipid, insulin, an inflammatory mediator, a lipid mediator, an insulin mediator and a cholesterol mediator; and (c) quantifying ribonucleic acids (RNA) in the biological fluid from liver, cardiovascular tissue, and kidney, wherein a threshold marker level and a threshold quantity of the RNA indicates the presence or increased risk of the complication in at least one of the liver, cardiovascular tissue and kidney. 66. The method of embodiment 65, wherein the at least one complication is selected from the group consisting of: NASH, liver fibrosis, liver cirrhosis, liver failure, diabetic nephropathy, renal ischemia, renal fibrosis, kidney failure, atherosclerosis, diabetic cardiomyopathy, atheroma, coronary artery disease, myocardial infarction, stroke and aneurysm. 67. The method of embodiment 65 or 66, wherein the chronic metabolic condition is selected from the group consisting of: obesity, type II diabetes and NAFLD. 68. The method of any one of embodiments 65-67, wherein the threshold quantity of RNA is substantially greater than that of at least one reference subject that does not have the at least one complication. 69. The method of any one of embodiments 65-68, wherein the threshold quantity of RNA does not differ substantially from that of at least one reference subject that has the at least one complication. 70. The method of any one of embodiments 65-69, comprising comparing the threshold quantity of RNA to respective reference levels, wherein the respective reference levels are average levels in a plurality of subjects suffering the at least one complication. 71. The method of any one of embodiments 65-70, wherein the threshold quantity of RNA being equal to or substantially greater than the average levels indicates the human subject suffers from the at least one complication. 72. The method of any one of embodiments 65-71, comprising detecting the complication when the threshold quantity of RNA is at least equal to or substantially greater than those of at least one subject with the at least one complication. 73. The method of any one of embodiments 65-72, wherein the biological fluid is selected from the group consisting of: plasma, urine and saliva. 74. The method of any one of embodiments 65-73, comprising measuring a marker level in whole blood and quantifying relative contributions of RNA in a plasma fraction of the whole blood. 75. The method of any one of embodiments 65-74, wherein the RNA is freely circulating RNA. 76. The method of any one of embodiment 65-75, wherein the inflammatory mediator is a cytokine. 77. The method of any one of embodiments 65-76, wherein the cholesterol mediator is a protein that mediates cellular uptake of cholesterol, cellular efflux of cholesterol, cholesterol metabolism, or modifications of cholesterol. 78. The method of any one of embodiments 65-77, wherein the lipid mediator is a mediator of lipid metabolism, lipid trafficking, lipid storage, or modifications of lipids. 79. The method of any one of embodiments 65-78, wherein RNA from kidney corresponds to a gene selected from the group consisting of: AK3L1, AQP2, AQPN6, ATP6V1G3, ATP6V0D2, BBOX1, BFSP2, BHMT, BSND, C20ORF194, C9orf66, CALB1, CA12, CDH16, CLCNKA, CRYAA, CRYBB3, CTXN3, CUBN, CYS1, DDC, DNMT3L, EGF, ENPEP, FCAMR, FMO1, FOLR3, FUT3, FXYD2, FXYD4, GGT1, HAO2, HAVCR1, HKID, HMX2, HNF1B, KAAG1, KCNJ1, KL, MCCD1, MIOX, NAT8, NOX4, NPHS2, OR2T10, PAX2, PDZK1, PDZK1IP1, PRR35, PTH1R, RBP5, SIM1, SLC12A1, SLC12A3, SLC13A3, SLC17A3, SLC22A11, SLC22A12, SLC22A13, SLC22A2, SLC22A24, SLC22A6, SLC22A8, SLC22A13, SLC34A1, SLC3A1, SLC4A9, SLC5A2, SLC5A10, SLC6A13, SLC6A18, SLC7A7, SLC7A8, SLC7A9, SOST, TREH, TMEM27, TMEM52B, TMEM72, TMEM174, TMEM207, UGT1A1, UGT1A6, UGT1A9, UMOD, UPP2, XPNPEP2, and 0001T8. 80. The method of any one of embodiments 65-80, wherein RNA from liver corresponds to a gene selected from the group consisting of: 1810014F10RIK, A1BG, ABCC2, ABCC6, ABCG5, ANG, ANGPTL3, ACOX2, ACSM2A, ADH1A, ADH1C, ADH6, AFM, AFP, AGXT, AHSG, AKR1C4, AKR1D1, ALB, ALDH1B1, ALDH4A1, ALDOB, AMBP, AOC3, APCS, APOA1, APOA2, APOA5, APOB, APOC1, APOC2, APOC3, APOC4, APOE, APOF, APOH, APOM, ARID1A, ARSE, ASL, AQP9, ASGR1, ASGR2, ATF5, C4A, C4BPA, C6, C8A, C8B, C8G, C9, CAPN5, CES1, CES2, CFHR1, CFHR2, CFHR3, CFHR4, CFHR5, CHD2, CIDEB, CPN1, CRLF1, CRYAA, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F12, DIO1, DAK, DCXR, F10, F12, F2, F9, FAH, FCN2, FETUS, FGA, FGB, FGG, FMO3, FTCD, G6PC, GPC3, GALK1, GAMT, GBA, GBP7, GCKR, GLYAT, GNMT, GPT, GSTM1, HAAO, HAMP, HAO1, HGD, HGFAC, HMGCS2, haptoglobin, HPN, HPR, HPX, HRG, HSD11B1, HSD17B6, HLF, IGF2, IL1RN, IGFALS, IQCE, ITIH1, ITIH2, ITIH4, JCLN, KHK, KLK13, LBP, LECT2, LOC55908, LPA, MASP2, MBL2, MGMT, MUPCDH, NHLH2, NNMT, NSFL1C, OATP1B1, ORM2, PCK1, PEMT, PGC, PLG, PKLR, PLGLB2, POLR2C, PON1, PON3, PROC, PXMP2, RBP4, RDH16, RET, SAA4, SARDH, SDS, SDSL, SEC14L2, SERPINA4, SERPINA7, SERPINA10, SERPINA11, SERPINC1, SERPIND1, SLCO1B1, SLC10A1, SLC22A1, SLC22A7, SLC22A10, SLC25A47, SLC27A5, SLC38A3, SLC6A12, SPP2, TAT, TBX3, TF, TIM2, TMEM176B, TST, UPB1, UROC1, VTN, WNT7A, C2, C2ORF72, CPB2, CYP4F11, CYP4F2, DUSP9, GABBR1, HP, HPD, IGSF1, IL17RB, ITIH2, ITIH3, LCAT, LGALS4, MAT1A, MST1, MSTP9, NR0B2, NR1I2, ORM1, RELN, RGN, RHBG, SAA4, SERPINA5, SERPINA7, SERPINC1, SERPINF2, SLC2A2, SULT1A2, SULT2A1, TCP10L, TNNI2, UGT2B15, and UGT2B17. 81. The method of any one of embodiments 65-80, wherein RNA from cardiovascular tissue corresponds to a gene selected from the group consisting of: ACTC1, ANKRD1, ASB18, BMP10, CASQ2, CCDC141, CHRNE, CORIN, CSRP3, DAND5, FABP3, GJA3, KLHL31, LRRC10, MT1HL1, MYBPC3, MYBPHL, MYH6, MYH7, MYL2, MYL3, MYL4, MYL7, MYOZ2, MYZAP, NPPA, NPPB, PLN, POPDC2, PPP1R1C, PRSS42, RD3L, RMB20, RYR2, SBK2, SBK3, SCN5A, SMCO1, ST8SIA2, TBX20 TECRL, TNNI3, TNNI3K, TNNT2, and XIRP1. 82. The method of any one of embodiments 65-81, wherein monitoring comprises performing steps a-c at least one time. 83. The method of any one of embodiments 65-82, monitoring comprises performing steps a-c at a first time point and a second time point. 84. The method of embodiment 83, wherein no presence or risk of complications are detected at the first time point. 85. The method of embodiment 83 or 84, wherein a presence or risk of at least one complication of at least one organ of the multiple organs is detected at the first time point, and the second time point occurs subsequent to an intervention or treatment of the complication. 86. A system comprising: (a) a memory unit configured to store results of (i) an assay for detecting at least one marker of each of at least one condition in a first sample of a subject, and (ii) an assay for detecting at least one tissue-specific RNA in a second sample of a subject, wherein each of the at least one tissue-specific RNA is a cell-free RNA specific to a tissue; (b) at least one processor programmed to: (i) quantify a level of the at least one marker; (ii) quantify a level of the at least one tissue-specific polynucleotide; (iii) compare the level of each of the at least one marker to a corresponding reference level of the marker; (iv) compare the level of each of the at least one tissue-specific polynucleotide to a corresponding reference level of the tissue-specific polynucleotide; and (v) determine presence of or relative change in damage of the tissue by the at least one condition based on the comparing; and (c) an output unit that delivers a report to a recipient, wherein the report provides results generated by the processor in (b). 87. The system of embodiment 86, wherein the report comprises a recommendation for medical action based on the generated by the processor in (b).

1. 88. The system of embodiment 87, wherein the medical action comprises recommended treatment. 89. The system of any one of embodiments 86-88, wherein the at least one tissue-specific polynucleotide comprises at least one tissue specific RNA. 90. The system of any one of embodiments 86-89, wherein the at least one tissue-specific polynucleotide comprises at least one tissue-specific methylated DNA, wherein each tissue-specific methylated DNA comprises a tissue-specific methylation pattern. 91. The system of any one of embodiments 86-90, wherein the tissue is determined to be damaged by the condition if (a) the level of at least one of the marker is above the reference level of the at least one marker, and (b) the level of at least one of the tissue-specific polynucleotide is above the reference level of the at least one tissue-specific polynucleotide. 92. The system of any one of embodiments 86-92, wherein the at least one condition is at least one of: inflammation, apoptosis, necrosis, fibrosis, infection, autoimmune disease, arthritis, liver disease, neurodegenerative disease, and cancer. 93. The system of any one of embodiments 86-91, wherein the at least one condition comprises multiple sclerosis. 94. The system of any one of embodiments 86-91, wherein the condition is inflammation, and the at least one marker corresponds to a gene selected from the group consisting of: AHSG, APCS, COX2, FAS, IL6, iNOS, OPN, ORM1, SIGIRR, SOCS3, TFN-alpha, and combinations thereof 95. The system of any one of embodiments 86-91, wherein the condition is fibrosis, and the at least one marker corresponds to a gene selected from the group consisting of: ALT, AST, C4M CPK, CO3-610, C06-MMP, C01-764, CTGF, IL-4, IL-6, IL-8, IL-18 MFAP, MMP1, MMP2, MMP9, MMP13, PDGF, PIIINP, PINP, P4NP 7S, PVCP, TGF-beta, TIMP1, TIMP2, TIMP3, TNF-alpha, YKL40, a gene encoding a troponin, and a gene encoding type IV collagen, and combinations thereof 96. The system of any one of embodiments 86-91, wherein the condition is apoptosis, and the at least one marker corresponds to a gene selected from the group consisting of: ALB, APAF1, APOE, CFLAR, CIDEB, F2, PLG, PROC, and TNFSF18, and combinations thereof 97. The system of any one of embodiments 86-91, wherein the condition is liver disease. 98. The system of embodiment 96, wherein the liver disease is non-alcoholic fatty liver disease, non-alcoholic steatosis, or non-alcoholic steatohepatitis. 99. The system of embodiment 98, wherein the liver disease is non-alcoholic fatty liver disease, and the method further comprises determining progress toward non-alcoholic steatohepatitis based on the results of step (b). 100. The system of any one of embodiments 86-91, wherein the at least one marker corresponds to a gene selected from the group consisting of: COX2, FAS, IL6, iNOS, LXR-alpha, OPN, PNPLA3 I148M, PPAR-gamma, SOCS3, SREBP-1c, SREBP-2, and TFN-alpha, and combinations thereof 101. The system of any one of embodiments 86-91, wherein the at least one marker is selected from the group consisting of: CRP, FIGF, HGF, ICAM1, IL2, IL2RA, IL8RB, KRT18, PI3, REG3A, ST2, TIMP1, TNFR, and TNFRSF1A, and combinations thereof 102. The system of any one of embodiments 86-101, wherein the at least one marker is cell-free RNA.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Identifying Markers and Marker Levels Indicative of NASH in Liver

Plasma samples are obtained from subjects in each of the following categories: (a) diagnosed with non-alcoholic steatohepatitis (NASH) and having moderate fibrosis; (b) diagnosed with NASH and having severe fibrosis; (c) age-matched normal subjects; (d) diagnosed with Hepatitis C viral infection (HCV) at an acute or early stage; (e) diagnosed with HCV and having a high level of fibrosis; and (f) diagnosed with alcoholic hepatitis. Markers or marker levels associated with NASH are detected and quantified for each sample. Example markers include cell-free mRNA and proteins encoded by genes selected from, but not limited to, LXR-alpha, PPAR-gamma, SREBP-1c, SREBP-2, FAS, iNOS, COX2, OPN, TNF-alpha, SOCS3, IL6, and PNPLA3 I148M. For general methods of detecting NASH-associated markers, or levels thereof, see e.g. Lima-Cabello et al., Clin Sci (Lond). 2011 March; 120(6):239-50 (incorporated herein by reference). Some markers, or quantities thereof, are used to distinguish between alternative sources of tissue damage. For example, SREBP-1c, SREBP-2 are elevated in NASH and non-alcoholic steatosis (NAS), but not significantly elevated in HCV without steatosis. For those markers exhibiting differential expression between the different subject groups, a reference or threshold level is selected, above which is diagnostic of NASH.

The plasma samples are also evaluated for levels of liver-specific cell-free RNA (cfRNA). cfRNA levels in the normal subjects serve as a baseline. Liver-specific cfRNA that is statistically significantly increased in the NASH subject samples are selected for use in testing subjects with an unknown condition. Examples of liver-specific genes include, without limitation, 1810014F10RIK, ACOX2, ACSM2A, ADH1A, ADH1C, AFM, AGXT, AKR1C4, AKR1D1, ALDH1B1, ALDH4A1, ALDOB, AMBP, APCS, APOA2, APOC1, APOC2, APOC4, APOF, ARID1A, ARSE, ASL, ATF5, C4A, C4BPA, C6, C8A, C8B, C8G, C9, CAPN5, CES1, CES2, CFHR1, CFHR4, CHD2, CPN1, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F12, DAK, DCXR, F10, F12, F2, FAH, FCN2, FETUS, FMO3, FTCD, G6PC, GALK1, GAMT, GBA, GCKR, GLYAT, GNMT, GPT, GSTM1, HAAO, HAMP, HAO1, HGD, HGFAC, HMGCS2, HPN, HPR, HPX, HRG, HSD11B1, HSD17B6, IGFALS, IQCE, ITIH1, ITIH4, JCLN, KHK, KLK13, LBP, LECT2, LOC55908, LPA, MASP2, MGMT, MUPCDH, NHLH2, NNMT, NSFL1C, OATP1B1, PCK1, PEMT, PKLR, POLR2C, PON1, PON3, PXMP2, RBP4, RDH16, RET, SARDH, SDS, SDSL, SEC14L2, SERPINA4, SERPIND1, SLC10A1, SLC22A1, SLC22A7, SLC27A5, SLC38A3, SLC6A12, TAT, TBX3, TF, TIM2, TMEM176B, TST, UPB1, VTN, WNT7A, ABCC2, ABCC6, ABCG5, ADH6, AHSG, ANG, ANGPTL3, AOC3, APOA1, APOC3, APOH, APOM, AQP9, ASGR1, ASGR2, C2, C2ORF72, CPB2, CYP4F11, CYP4F2, DUSP9, GABBR1, HP, HPD, IGSF1, IL17RB, ITIH2, ITIH3, LCAT, LGALS4, MAT1A, MST1, MSTP9, NR0B2, NR1I2, ORM1, PROC, RELN, RGN, RHBG, SAA4, SERPINA5, SERPINC1, SERPINF2, SULT1A2, SULT2A1, TCP10L, UGT2B15, UGT2B17, AFP, ALB, APOB, CRLF1, CRYAA, DIO1, GPC3, HLF, IGF2, IL1RN, PGC, SERPINA7, SLC2A2, TNNI2, ALB, APOE, CIDEB, F2, PLG, and PROC. General methods for detecting cfRNA are provided in US20130252835, which is incorporated herein by reference. Increased levels of transcript fragments from at least one of these genes may be used as an indicator of increased liver tissue damage, and hepatocyte damage in particular.

### Example 2: Diagnosing NASH in Liver

A plasma sample is obtained from a subject. One aliquot of the sample is tested to determine the level of markers associated with NASH, as in Example 1. A second aliquot of the sample is tested to determine the level of liver-specific cfRNAs. If the subject has a marker level above a threshold, and a cfRNA level above a threshold, the subject is diagnosed as having NASH. If the subject has a marker level above a threshold, but a cfRNA level at or below the threshold, the subject is diagnosed as not having NASH. A diagnosis of NASH by this method has a higher accuracy and specificity than a diagnostic based on the markers alone, which in the absence of increased liver-specific cfRNA may instead indicate inflammation in another tissue.

If the subject is diagnosed as having NASH, the subject undergoes treatment for the condition. The method is then repeated to track therapeutic efficacy, indicated by a decrease in the level of at least one of the markers and/or at least one of the liver-specific cfRNAs.

### Example 3: Diagnosing, Treating and Monitoring Liver Disease

A plasma sample is obtained from a subject. The sample is tested to determine the level of markers for inflammation, apoptosis, and fibrosis, as well as for tissue-specific cell-free RNAs from the liver and other tissues (e.g. kidney, and lungs). The tests are performed on the same aliquot or on different aliquots of the sample. The subject is diagnosed as having liver disease if: (a) the markers are above a reference level (indicating presence of the conditions); (b) liver-specific cell-free RNAs are above a reference level (indicating liver damage); and (c) tissue-specific cell-free RNAs from the non-liver tissues are not above a reference level (indicating that those non-liver tissues are not undergoing the inflammation, apoptosis, and fibrosis). Liver damage is verified by measuring an increase in the level of markers for liver damage, including plasma protein genes.

The subject is treated for liver disease, such as with a pharmaceutical composition. The tests are repeated to determine therapeutic efficacy, indicated by a decrease in the level of at least one of the markers and/or at least one of the liver-specific cfRNAs. Optionally, at least one target of the pharmaceutical composition, and/or at least one downstream member of a signaling pathway comprising the at least one target are also assessed before and after treatment to determine whether the pharmaceutical composition has the desired activity in the subject. For example, a pharmaceutical composition comprising an inhibitor of a target protein would be expected to reduce activity of the target protein, as well as reduce the expression of any genes that are positively regulated (directly or indirectly) by the target protein.

### Example 4: Clinical Utility of Noninvasive Methods

A 55 year old patient has been obese for most of his life and experiences some dull pain in the right upper quadrant of his abdomen. A number of conditions can cause pain in this area, including NAFLD, NASH, gallstones and inflammation. The patient's primary care physician suspects the patient may be suffering from NASH due to a family history of cirrhosis, but a liver biopsy would be necessary to confirm the suspicion. The patient's primary care physician is hesitant to perform a biopsy, which risks an infection, without a further indication that the patient is really suffering from NASH. Before performing a liver biopsy, the patient's primary care physician orders a test that quantifies liver-specific cfRNAs and markers of liver diseases in a plasma sample of the patient. The liver-specific cfRNAs will indicate if the liver is being affected, and markers of disease may reveal which condition is causing the pain. The results indicate that the levels of markers and liver-specific polynucleotides are most similar to that of a subject with NASH, as determined in Example 1. The results indicate that the patient has liver damage but is not suffering from NASH. Instead marker levels indicate that the patient suffers from NAFLD. The patient is prescribed a bile acid analog. The patient is also now more determined to stick to a low-calorie diet. The patient loses some weight and takes the bile acid analog. A year later, the test is performed again. Levels of liver-specific cfRNAs and markers of NAFLD are reduced in the patient plasma sample relative to levels observed with the first test. The client does not develop NASH or cirrhosis and never receives a liver biopsy. This Example demonstrates the benefit to the public of offering a noninvasive liver health assay that is both sensitive and specific, and carries low risk to the patient's health.

### Example 5: Differentiating Between NAFLD and NASH

Plasma samples are obtained from four overweight subjects. Cholesterol, triglycerides and CRP levels are measured in all four subjects. In Subjects 1 and 2, cholesterol, triglycerides and CRP levels are similar to normal, healthy individuals. In Subjects 3 and 4, cholesterol, triglycerides and CRP levels are higher than normal, healthy individuals, and similar to those of NASH patients.

In addition, to cholesterol, triglycerides and CRP levels, liver-specific RNA in the plasma samples is quantified relative to total RNA of all subjects' plasma samples. In Subjects 1 and 3, liver-specific RNA levels are similar to that of normal, healthy individuals, and similar to those of NASH patients. In Subjects 2 and 4, liver-specific RNA levels are higher than that of normal, healthy individuals, and similar to those of NASH patients. The following inferences are drawn: Subject 1 is not likely to have developed NAFLD or NASH; Subject 2 is not likely to have developed NAFLD or NASH, but has another liver condition; Subject 3 has NAFLD, but not NASH; Subject 4 has NAFLD that has progressed to NASH.

### Example 6: Identifying Markers and Marker Levels Indicative of Ovarian Cancer

Plasma samples are obtained from subjects in each of the following categories: (a) menopausal; (b) diagnosed with benign ovarian (fibrotic) cysts; (c) diagnosed with endometriosis; (d) diagnosed ovarian cancer in early stages; (e) diagnosed with ovarian cancer at late stages; (f) diagnosed with uterine cancer; and (g) diagnosed with breast cancer. Markers or marker levels and levels of ovarian-specific RNA associated with ovarian conditions are detected and quantified for each sample. Example markers include cell-free mRNA and proteins encoded by genes selected from, but not limited to, glycoprotein C125, TAG-72, CA15-3, OVX1, M-CSF, CEA, IL-6, AFP, beta-hCG, HE4, BRCA1, BRCA2, inhibin A and inhibin B. For those markers exhibiting differential expression between the different subject groups, reference or threshold levels are selected, above which is diagnostic of early and late stage ovarian cancer.

The plasma samples are also evaluated for levels of ovary-specific cell-free RNA (cfRNA). cfRNA levels in the normal subjects serve as a baseline. Ovary-specific cfRNA that is statistically significantly increased in the ovarian cancer subject samples are selected for use in testing subjects with an unknown condition. Examples of ovary-specific polynucleotides, and levels thereof, include, without limitation, those encoded by genes selected from ANGPTL5, ARX, C/EBP-delta, CRYGD, ECEL1, GRO-alpha, GRO-beta, HIN-1, IK-alpha, IL-8, KLHDC8A, LIF, M1S1, MIP3-alpha, MMP10, MMP26, MUM1L1, PRP, RASD1, RP4-559A3.7, RPS6, SOD2, TM4SF1, TNFAIP2 TRH, and WFIKKN2. Increased levels of transcript fragments from at least one of these genes may be used as an indicator of ovary damage, which may be caused by ovarian cancer.

### Example 7: Diagnosing Ovarian Caner

A plasma sample is obtained from a subject. One aliquot of the sample is tested to determine the level of markers associated with ovarian cancer, as in Example 1. A second aliquot of the sample is tested to determine the level of ovary-specific cfRNAs. If the subject has a marker level above a threshold, and a cfRNA level above a threshold, the subject is diagnosed as having ovarian cancer. If the subject has a marker level above a threshold, but a cfRNA level at or below the threshold, the subject is not diagnosed with ovarian cancer. A diagnosis of ovarian cancer by this method has a higher accuracy and specificity than a diagnostic based on the markers alone, which in the absence of increased ovary-specific cfRNA may instead indicate a condition, such as inflammation, in another tissue.

If the subject is diagnosed as having ovarian cancer, the subject undergoes treatment for the condition. The method is then repeated to track therapeutic efficacy, indicated by a decrease in the level of at least one of the markers and/or at least one of the ovary-specific cfRNAs.

### Example 8: Diagnosing, Treating and Monitoring Ovarian Cancer

A plasma sample is obtained from a subject. The sample is tested to determine the level of markers for inflammation, apoptosis, and fibrosis, as well as for tissue-specific cell-free RNAs from the ovary and other tissues (e.g. bladder and uterus). The tests are performed on the same aliquot or on different aliquots of the sample. The subject is diagnosed as having ovarian cancer if: (a) the markers are above a reference level (indicating presence of the conditions); (b) ovary-specific cell-free RNAs are above a reference level (indicating ovary damage); and (c) tissue-specific cell-free RNAs from the non-ovary tissues are not above a reference level (indicating that those non-ovary tissues are not undergoing the inflammation, apoptosis, and fibrosis). Liver damage is verified by measuring an increase in the level of markers for ovary damage, including plasma protein genes.

The subject is treated for ovarian cancer, such as with a pharmaceutical composition. The tests are repeated to determine therapeutic efficacy, indicated by a decrease in the level of at least one of the markers and/or at least one of the ovary-specific cfRNAs. Optionally, at least one target of the pharmaceutical composition, and/or at least one downstream member of a signaling pathway comprising the at least one target are also assessed before and after treatment to determine whether the pharmaceutical composition has the desired activity in the subject. For example, a pharmaceutical composition comprising an inhibitor of a target protein would be expected to reduce activity of the target protein, as well as reduce the expression of any genes that are positively regulated (directly or indirectly) by the target protein.

### Example 9: Clinical Utility of Noninvasive Methods for Cancer

A 55 year old woman is menopausal and experiencing abnormally frequent blood spotting. A number of conditions can cause spotting and is sometimes just a regular occurrence during menopause. However, spotting can also occur when the subject has ovarian or uterine cysts or tumors. The patient's primary care physician is concerned that the patient may have an ovarian tumor based on a family history cancer. The physician could perform an ultrasound or other scanning imaging test. However, a negative result may mean that the cancer is only in early stages and thus, tumors are too small to detect in an image. Instead, the patient's primary care physician orders a test that quantifies ovary-specific cfRNAs and markers of ovarian cancer in a plasma sample of the patient. The ovary-specific cfRNAs will indicate if an ovary is being affected and markers of disease may reveal if the patient has a tumor. The results indicate that the levels of markers and ovary-specific polynucleotides are most similar to that of a subject with early stages of ovarian cancer, as determined in Example 5. Thus, the test results indicate that the patient has early stages of ovarian cancer. The patient opts to have a hysterectomy. The test is repeated every three months. Levels of ovary-specific cfRNAs and markers of ovarian cancer are reduced in the patient plasma sample relative to levels observed with the first test. A year later, an ultrasound is performed and there is no evidence of ovarian tumors in the ultrasound images. The client's ovaries never develop visible tumors and it is determined that the ovarian cancer cells have failed to proliferate in the absence of hormones and stimuli from the uterus. This Example demonstrates the benefit to the public of offering a means to detect ovarian cancer at early stages and differentiate early stages of ovarian cancer from other conditions. One skilled in the art would easily understand how these tests may similar be used for other cancers, such as colon cancer where rectal bleeding may be mistaken for internal hemorrhoids.

### Example 10: Routine Screening of Obese Patients for Heart Attack, Liver Fibrosis and Kidney Failure

Several subjects with a body mass index (BMI) greater than 30 get an annual heath examination by their physician. The physician informs the subjects that they are at an increased risk of a heart attack, liver fibrosis (e.g. NASH), and kidney failure because of her weight. The physician suggests a non-invasive test that will let them know if any one of these conditions are present or imminent. The subjects agree to the test and go to a lab where a blood sample is taken. The lab obtains a plasma sample from a portion of their blood samples. The lab tests the blood for markers such as cholesterol levels, triglyceride levels, white blood cell count and levels of a few inflammatory markers, such as C reactive protein (CRP). In addition, cell-free nucleic acids in the plasma sample (RNA and optionally, methylated DNA) corresponding to genes highly expressed in cardiovascular tissue (e.g., coronary artery and aorta), liver and kidney are quantified.

A first subject has elevated levels of cholesterol and CRP, but does not have elevated levels of cell-free nucleic acids corresponding to genes highly expressed in cardiovascular tissue, liver and kidney relative to a non-obese, healthy subject without cardiovascular, liver or kidney conditions. The first subject is prescribed a statin and daily low dose aspirin. The first subject remains overweight most of their life, but never develops a life-threatening condition of their liver, kidney or cardiovascular system. The physician orders the test for the first subject at least once a year to ensure the first subject's health status has not changed and that the prescribed treatment remains effective. Alternatively, the first subject does not have the test, is not prescribed a statin and low dose aspirin. The first subject develops arterial plaques and eventually succumbs to stroke.

A second subject has elevated levels of cholesterol and CRP, as well as elevated levels of cell-free nucleic acids corresponding to genes highly expressed in cardiovascular tissue relative to a non-obese, healthy subject without a cardiovascular condition. A coronary angiogram of the second subject is obtained and their coronary artery is shown to be partially occluded. Necrosis of an arterial plaque has begun and a thrombotic rupture is imminent. An angioplasty or artery stent is administered to the second subject before a heart attack occurs. The second subject is also prescribed a statin and daily low dose aspirin. The physician orders the test for the second subject at least once a year to ensure the second subject's health status has not changed and that the prescribed treatment remains effective. Alternatively, the second subject does not have the test, does not receive an angioplasty or stent and experiences a heart attack four weeks later.

A third subject has been extremely obese for many years. Surprisingly, the third subject does not have elevated levels of cholesterol. However, the third subject has levels of inflammatory markers and elevated levels of cell-free nucleic acids corresponding to genes highly expressed in liver and kidney that are similar to corresponding levels in reference subjects with NASH and diabetic nephropathy respectively. A liver biopsy is performed, and cirrhosis of the liver is observed. In addition, albumin in urine of the third subject is measured and urinary albumin excretion is found to be 200 mg in a 24 h period. The third subject gains awareness that they may have multi-organ failure if they do not take some drastic measures. The subject undergoes gastric bypass surgery and begins a treatment regimen with an acetylcholinesterase (ACE) inhibitor. The subject loses weight and does not need dialysis or a liver transplant. The physician orders the test for the third subject at least once a year to ensure the third subject's health status has not changed and that the prescribed treatment remains effective. Alternatively, the subject does not receive the test and does not receive a gastric bypass surgery or an ACE inhibitor. The subject eventually receives a new liver, but finally succumbs to kidney failure.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-64. (canceled)

65. A method of detecting a cardiovascular disease (CVD) biosignature in a biological fluid from a human subject, comprising:
(a) measuring a marker level in the biological fluid, wherein the marker is selected from a cholesterol, a lipid, an inflammatory mediator, a lipid mediator, and a sterol mediator; and
(b) quantifying a quantity of cardiovascular ribonucleic acids (RNA) in the biological fluid, wherein a threshold marker level and a threshold quantity of cardiovascular RNA indicates the CVD biosignature.

66. A method of detecting a non-alcoholic steatohepatitis (NASH) biosignature in a biological fluid from a human subject, comprising:
(a) measuring a marker level in the biological fluid, wherein the marker is selected from a cholesterol, a lipid, an inflammatory mediator, a lipid mediator, and a cholesterol mediator; and
(b) measuring a quantity of liver ribonucleic acids (RNA) in the biological fluid, wherein a threshold marker level and a threshold quantity of liver RNA indicates the NASH biosignature.

67. The method of claim 66, wherein the marker comprises at least one polynucleotide or protein encoded by a gene selected from the group consisting of: LXR-alpha, PPAR-gamma, SREBP-1c, SREBP-2, FAS, iNOS, COX2, OPN, TFN-alpha, SOCS3, IL6, and PNPLA3 I148M.

68. The method of claim 66, wherein the cholesterol mediator comprises a polynucleotide or protein encoded by a gene selected from the group consisting of: LXR-alpha, SREBP-1c, and SREBP-2.

69. The method of claim 66, wherein the inflammatory mediator comprises a polynucleotide or protein encoded by a gene selected from the group consisting of: iNOS, COX2, OPN, TFN-alpha, SOCS3 and IL-6.

70. The method of claim 66, wherein the lipid mediator comprises a polynucleotide or protein encoded by a gene selected from the group consisting of: PPAR-gamma, FAS, and PNPLA3 I148M.

71. The method of claim 66, wherein measuring the quantity of liver RNA comprises measuring the relative contribution of liver RNA to a nucleic acid population selected from total RNA of the biological fluid and total nucleic acids of the biological fluid.

72. The method of claim 66, wherein the liver RNA does not encode proteins implicated in NASH.

73. The method of claim 66, wherein the liver RNA does not encode proteins upregulated in a liver of a reference subject with NASH.

74. The method of claim 66, further comprising obtaining a second biological fluid, and detecting a NASH biosignature in the second biological fluid.

75. The method of claim 74, wherein the second biological fluid is obtained subsequent to a NASH intervention.

76. The method of claim 75, wherein the NASH intervention comprises at least one of: reducing alcohol intake, reducing caloric intake, increasing exercise, undergoing gastric bypass surgery, reducing cholesterol level, reducing inflammation, and improving insulin sensitivity.

77. The method of claim 75, wherein the NASH intervention comprises consuming a compound selected from a cholesterol-regulating compound, an anti-inflammatory compound, and an insulin sensitizing compound.

78. The method of claim 66, wherein the liver RNA corresponds to a gene selected from the group consisting of: 1810014F10RIK, A1BG, ABCC2, ABCC6, ABCGS, ANG, ANGPTL3, ACOX2, ACSM2A, ADH1A, ADH1C, ADH6, AFM, AFP, AGXT, AHSG, AKR1C4, AKR1D1, ALB, ALDH1B1, ALDH4A1, ALDOB, AMBP, AOC3, APCS, APOA1, APOA2, APOAS, APOB, APOC1, APOC2, APOC3, APOC4, APOE, APOF, APOH, APOM, ARID1A, ARSE, ASL, AQP9, ASGR1, ASGR2, ATFS, C4A, C4BPA, C6, C8A, C8B, C8G, C9, CAPNS, CES1, CES2, CFHR1, CFHR2, CFHR3, CFHR4, CFHRS, CHD2, CIDEB, CPN1, CRLF1, CRYAA, CYP1A2, CYP27A1, CYP2A13, CYP2A6, CYP2A7, CYP2B6, CYP2C19, CYP2C8, CYP2C9, CYP2D6, CYP2E1, CYP3A4, CYP4A11, CYP4A22, CYP4F12, DIO1, DAK, DCXR, F10, F12, F2, F9, FAH, FCN2, FETUB, FGA, FGB, FGG, FMO3, FTCD, G6PC, GPC3, GALK1, GAMT, GBA, GBP7, GCKR, GLYAT, GNMT, GPT, GSTM1, HAAO, HAMP, HAO1, HGD, HGFAC, HMGCS2, haptoglobin, HPN, HPR, HPX, HRG, HSD11B1, HSD17B6, HLF, IGF2, IL1RN, IGFALS, IQCE, ITIH1, ITIH2, ITIH4, JCLN, KHK, KLK13, LBP, LECT2, LOC55908, LPA, MASP2, MBL2, MGMT, MUPCDH, NHLH2, NNMT, NSFL1C, OATP1B1, ORM2, PCK1, PEMT, PGC, PLG, PKLR, PLGLB2, POLR2C, PON1, PON3, PROC, PXMP2, RBP4, RDH16, RET, SAA4, SARDH, SDS, SDSL, SEC14L2, SERPINA4, SERPINA7, SERPINA10, SERPINA11, SERPINC1, SERPIND1, SLCO1B1, SLC10A1, SLC22A1, SLC22A7, SLC22A10, SLC25A47, SLC27A5, SLC38A3, SLC6A12, SPP2, TAT, TBX3, TF, TIM2, TMEM176B, TST, UPB1, UROC1, VTN, WNT7A, C2, C2ORF72, CPB2, CYP4F11, CYP4F2, DUSP9, GABBR1, HP, HPD, IGSF1, IL17RB, ITIH2, ITIH3, LCAT, LGALS4, MAT1A, MST1, MSTP9, NR0B2, NR1I2, ORM1, RELN, RGN, RHBG, SAA4, SERPINA5, SERPINA7, SERPINC1, SERPINF2, SLC2A2, SULT1A2, SULT2A1, TCP10L, TNNI2, UGT2B15, and UGT2B17.

79. The method of claim 66, further comprising measuring a quantity of a deoxyribonucleic acid (DNA) in the biological fluid, wherein the DNA has a liver methylation pattern of at least one locus.

80. The method of claim 79, wherein measuring the quantity of the DNA having a liver methylation pattern of at least one locus comprises measuring the relative contribution of DNA having a liver methylation pattern of at least one locus to total DNA in the biological fluid.

81. The method of claim 80, wherein the at least one locus of the methylated DNA is not implicated in NASH.

82. The method of claim 79, further comprising sequencing at least one DNA and at least one RNA in the biological fluid.

83. The method of claim 66, wherein the biological fluid is plasma or serum.

84. The method of claim 66, wherein the liver RNA is freely circulating RNA.
